(19) United States
(12) Patent Application Publication
(10) Pub. No.: US 2025/0000812 A1
(43) Pub. Date: Jan. 2, 2025

(54) NANO-SUSPENSIONS AND AMORPHOUS SOLID DISPERSIONS OF HYDROPHOBIC AGENTS AND METHODS OF USE THEREOF

(71) Applicant: Genentech, Inc., South San Francisco, CA (US)

(72) Inventor: Dennis Hengho LEUNG, South San Francisco, CA (US)

(73) Assignee: Genentech, Inc., South San Francisco, CA (US)

(21) Appl. No.: 18/886,607

(22) Filed: Sep. 16, 2024

Related U.S. Application Data
(63) Continuation of application No. PCT/US2023/064668, filed on Mar. 17, 2023.
(60) Provisional application No. 63/321,516, filed on Mar. 18, 2022.
(60) Provisional application No. 63/321,519, filed on Mar. 18, 2022.

(51) Int. Cl. A61K 9/51; A61K 31/192; A61K 31/343

(52) U.S. Cl. CPC A61K 9/5192; A61K 9/5123; A61K 9/5161; A61K 31/192; A61K 31/343

(57) ABSTRACT

Described herein, in certain aspects, are nano-suspensions comprising a plurality of nanoparticles comprising a hydrophobic agent suspended in an aqueous carrier having a desired viscosity. In other aspects, the described herein are methods of use thereof, including methods of making a dosage form, components of the nano-suspensions, dispensing cartridges and composition therein, dosage forms, devices for dispensing a nano-suspension, and system and applications of the embodiments described herein.

### CROSS-REFERENCE TO RELATED APPLICATION

This application claims priority to and the benefit of U.S. Provisional Patent App. Nos. 63/321,516 and 63/321,519, filed Mar. 18, 2022, which are incorporated herein by reference herein in its entirety.

### TECHNICAL FIELD

The present disclosure, in certain aspects, is directed to nano-suspensions comprising a plurality of nanoparticles comprising a hydrophobic agent suspended in an aqueous carrier having a desired viscosity. In other aspects, the present disclosure is directed to amorphous solid dispersions (ASDs) comprising an agent susceptible to crystallization. Also provided are associated methods (such as methods of identifying ASD forming solutions, and methods of making, e.g., methods of making a making dosage form comprising the hydrophobic agent and/or the agent susceptible to crystallization), compositions (e.g., for use in the methods of making and compositions produced therefrom), devices, and systems.

### BACKGROUND

Traditionally, the development of drug products has often taken a “one-size-fits-all” approach with respect to dosage and image, most commonly exemplified by standard tablet sizes. This requires the development of a suitable bulk manufacturing approach and supply chain with the appropriate quality control in order to deliver the drug product at the specific doses to the target patient population. However, increasing understanding of disease pathology and its relationship with genetics as well as an increased amount of statistical data that can be obtained for each patient has driven significant interest in personalized medicine approaches. Furthermore, the rise of highly potent drug molecules has placed an emphasis on precise control over the delivery of small quantities of drug. Additionally, the pharmaceutical industry has been challenged by the increasing number of poorly soluble drug candidates. This can cause significant issues with obtaining sufficient absorption and bioavailability, risk of exposure variability, and difficulties in maintaining a safe therapeutic index. Current manufacturing approaches for solid dosage forms such as tablets face significant challenges with consistency and content uniformity for low dosages, which are particularly important for highly potent drugs as well as for pediatric indications. As a result, the ability to manufacture a precise dosage forms on demand which can be tailored for an individual patient while handling low quantities of drug has significant value.

The crystalline form of an agent, such as a hydrophobic therapeutic drug, may undesirably impact properties such as bioavailability, dissolution rate, solubility, and stability. This can cause significant issues with obtaining sufficient absorption and bioavailability, risk of exposure variability, and difficulties in maintaining a safe therapeutic index. Discovery and use of non-crystalline solid forms, e.g., as an amorphous solid dispersion, of such agents can enable more predictable and efficacious therapeutic use. Amorphous solid dispersions are a combination including a ratio of an agent susceptible to formation of a crystalline form and a polymer, wherein the agent is maintained in a non-crystalline form. Amorphous solid dispersions provide agents in a form having high solubility and sustained supersaturation. The identification and development of amorphous solid dispersions is challenging, costly, and requires large amounts of raw materials. In drug development contexts, such demands hinder progress and the identification of useful drug dosage forms, especially when only small amounts of a drug compound are available.

### BRIEF SUMMARY

The present disclosure is directed to methods of making a dosage form comprising a hydrophobic agent. The methods comprise depositing a plurality of droplets of a formulation comprising the hydrophobic agent onto a substrate using a high frequency droplet dispenser to form a dosage form. In certain aspects, the viscosity of the formulation is less than 3 cP.

In some embodiments, the high frequency droplet dispenser deposits the plurality of droplets using a thermal process. In some embodiments, the high frequency droplet dispenser deposits the plurality of droplets using a piezoelectric process. In some embodiments, the high frequency droplet dispenser comprises about 100 to about 1000 individual nozzles. In some embodiments, each nozzle comprises an aperture with a diameter of about 20 μm to about 120 μm. In some embodiments, the high frequency droplet dispenser is a high frequency, sub-nanoliter droplet dispenser. In some embodiments, the high frequency, sub-nanoliter droplet dispenser dispenses droplets at more than 3 kHz as measured per nozzle. In some embodiments, the high frequency droplet dispenser dispenses droplets with a volume of 1 nanoliter or less.

In some embodiments, the average volume of each dispensed droplet of the plurality of droplets of the formulation is about 1 picoliter to about 50 picoliters.

In some embodiments, the plurality of formulation droplets are deposited onto the substrate according to an average number of droplets per square inch. In some embodiments, the average number of droplets deposited per square inch of the substrate is about 1 droplet to about 500 droplets per square inch.

In some embodiments, the plurality of droplets of the formulation deposited on the substrate have an average diameter of about 5 μm to about 100 μm.

In some embodiments, the plurality of droplets of the formulation deposited on the substrate form a continuous layer.

In some embodiments, the viscosity of the formulation is 0.5 cP to 3 cP. In some embodiments, the viscosity of the formulation is less than 2 cP and greater than 1 cP. In some embodiments, the viscosity of the formulation is between 1.2 cP to 1.8 cP.

In some embodiments, the formulation further comprises a viscosity-modulating agent. In some embodiments, the viscosity-modulating agent is a viscosity-enhancing agent. In some embodiments, the viscosity-enhancing agent is selected from the group consisting of ethylene glycol, polyethylene glycol, glycerol, and propylene glycerol. In some embodiments, the viscosity-modulating agent is a viscosity-reducing agent. In some embodiments, the viscosity-reducing agent is a salt or an amino acid, or a combination thereof. In some embodiments, the amount of the viscosity-modulating agent in the mixture is based on a desired viscosity of the mixture. In some embodiments, the viscosity-enhancing agent is present in the formulation at a concentration of about 1% to about 40% v/v of the formulation. In some embodiments, further comprising adding the viscosity-enhancing agent to the formulation to achieve a viscosity of the formulation of between about 1 cP and about 2 cP.

In some embodiments, the formulation is a suspension comprising: an aqueous carrier, and a plurality of nanoparticles suspended in said aqueous carrier, wherein said nanoparticles comprise the hydrophobic agent. In some embodiments, the suspension is a nanosuspension. In some embodiments, the suspension is a colloidal suspension.

In some embodiments, the aqueous carrier comprises water and an organic solvent.

In some embodiments, the ratio of the viscosity-enhancing agent to the aqueous carrier of the formulation is from about 1:1000 to about 1:2 by volume.

In some embodiments, the hydrophobic agent is not soluble in the aqueous carrier. In some embodiments, the hydrophobic agent has a solubility in water of less than 1 mg/mL.

In some embodiments, the concentration of the hydrophobic agent in the suspension is about 5 mg/mL to about 1,000 mg/mL.

In some embodiments, the suspension further comprises a stabilizing component. In some embodiments, the ratio of the stabilizing component to the aqueous carrier of the suspension is about 1:500 to about 1:10 by weight. In some embodiments, the stabilizing component comprises PVP and/or SDS.

In some embodiments, the average diameter of the plurality of nanoparticles is less than about 500 nm. In some embodiments, the plurality of nanoparticles has a D50 of about 100 nm to about 400 nm. In some embodiments, the plurality of nanoparticles has a D90 of about 250 μm to about 50 nm.

In some embodiments, at least a portion of the plurality of nanoparticles is deposited within the substrate. In some embodiments, at least a portion of the plurality of nanoparticles is deposited on top of the substrate.

In some embodiments, the formulation is an amorphous solid dispersion (ASD) forming solution comprising: the hydrophobic agent; an organic carrier; and a polymer, wherein the ratio of the polymer and the hydrophobic agent inhibits crystallization of the hydrophobic agent in a dried state on the substrate. In some embodiments, the ASD forming solution is a colloid.

In some embodiments, the organic carrier of the ASD forming solution comprises any one or more components selected from the group consisting of acetone, dimethylformamide, ethanol, methanol, pentane, hexane, heptane, ethyl acetate, isopropyl alcohol, ethylene glycol, and dimethyl sulfoxide.

In some embodiments, the ASD forming solution comprises at least 30% of an organic carrier by volume.

In some embodiments, the viscosity-modulating agent has a ratio to the organic carrier of 1:100 to 40:100 by volume.

In some embodiments, the polymer of the ASD forming solution is suitable for formation of an amorphous solid dispersion (ASD) following deposition by the droplet dispenser. In some embodiments, the polymer is selected based on one or more characteristics of the hydrophobic agent. In some embodiments, the polymer is selected from the group consisting of a cellulose derivative, poly(vinyl pyrrolidinone) (PVP), poly(vinyl pyrrolidinone-co-vinyl acetate) (copovidone, or PVPVA), hydroxypropyl methyl cellulose (hypromellose, or HPMC), and HPMC acetate succinate (hypromellose acetate succinate or HPMC-AS), and eudragit, or any combination thereof. In some embodiments, the polymer is a grade selected from the group consisting of LF, MF, HF, LMP, MMP, HMP, LG, MG, and HG. In some embodiments, the polymer is HPMC-AS-LF. In some embodiments, the ratio of the concentration of the polymer to the concentration of the hydrophobic agent is from 1:20 to 20:1. In some embodiments, ratio of the concentration of the polymer to the concentration of the hydrophobic agent is 1:2 to 2:1.

In some embodiments, the ASD forming solution further comprises a surfactant. In some embodiments, the surfactant is selected from Tween 80, Vit-E, TPGS, or SDS, or any combination thereof. In some embodiments, the ASD forming solution comprises 10% to 20% of the surfactant by volume. In some embodiments, the ASD forming solution comprises a ratio of the surfactant to the hydrophobic agent of 1:100 to 5:1.

In some embodiments, less than 5% of the hydrophobic agent crystallizes in a dried state following deposition by the droplet dispenser. In some embodiments, substantially no crystallization of the hydrophobic agent occurs in a dried state.

In some embodiments, the ASD forming solution is supersaturated with the hydrophobic agent. In some embodiments, the concentration of the hydrophobic agent in the mixture is about 5 mg/mL to about 1,000 mg/mL.

In some embodiments, at least a portion of the hydrophobic agent is within the substrate following deposition by the droplet dispenser. In some embodiments, at least a portion of the hydrophobic agent is on top of the substrate following deposition by the droplet dispenser.

In some embodiments, the hydrophobic agent is a therapeutic agent. In some embodiments, the therapeutic agent is a small molecule drug. In some embodiments, the therapeutic agent is a biomolecule. In some embodiments, the therapeutic agent is a Biopharmaceutics Classification System (BCS) class II or class IV agent. In some embodiments, the therapeutic agent has a narrow therapeutic index. In some embodiments, the therapeutic agent is a high-potency drug. In some embodiments, the hydrophobic agent is an excipient or an adjuvant.

In some embodiments, the hydrophobic agent is a diagnostic compound.

In some embodiments, the hydrophobic agent is a reagent.

In some embodiments, the hydrophobic agent has a solubility in water of less than 0.1 mg/mL.

In some embodiments, the amount of the hydrophobic agent is less than 0.1 mg.

In some embodiments, the substrate is a pharmaceutically acceptable polymer.

In some embodiments, the substrate is selected from the group consisting of one or more of chitosan, ethylenediaminetetraacetic acid, polyvinyl pyrrolidone, polyvinyl alcohol, alginate, agar, carrageenan, guar gum, xanthan gum, polycarbophil, and polyacrylic acid derivatives. In some embodiments, the substrate is a thin film substrate.

In some embodiments, the dosage form is an oral dosage form. In some embodiments, the dosage form is a buccal dosage form. In some embodiments, the dosage form is a transdermal dosage form. In some embodiments, the dosage form is sterile.

In other aspects, provided herein is a suspension comprising: an aqueous carrier; a plurality of nanoparticles comprising a hydrophobic agent suspended in the aqueous carrier, wherein the viscosity of the suspension is less than 3 cP.

In some embodiments, the viscosity of the suspension is less than 2 cP and greater than 1 cP. In some embodiments, the viscosity of the suspension is between 1.2 cP to 1.8 cP.

In some embodiments, the suspension further comprises a viscosity-modulating agent. In some embodiments, the viscosity-modulating agent is a viscosity-enhancing agent. In some embodiments, the viscosity-enhancing agent is selected from the group consisting of ethylene glycol, polyethylene glycol, glycerol, and propylene glycerol.

In some embodiments, the viscosity-enhancing agent is present in the suspension at a concentration of about 1% to about 40% v/v of the suspension. In some embodiments, the ratio of the viscosity enhancing agent to the aqueous carrier of the suspension is about 1:1000 to about 1:2 by volume. In some embodiments, further comprising adding the viscosity-enhancing agent to the suspension to achieve a viscosity of the suspension of between about 1 cP and about 2 cP.

In some embodiments, the aqueous carrier comprises water and an organic solvent.

In some embodiments, the suspension is a colloidal suspension.

In some embodiments, the suspension further comprises a stabilizing component. In some embodiments, the ratio of the stabilizing component to the aqueous carrier of the suspension is about 1:500 to about 1:10 by weight. In some embodiments, the stabilizing component comprises PVP and/or SDS.

In some embodiments, the average diameter of the plurality of nanoparticles is less than about 500 nm. In some embodiments, the plurality of nanoparticles has a D50 of about 100 nm to about 400 nm. In some embodiments, the plurality of nanoparticles has a D90 of about 250 μm to about 50 nm.

In some embodiments, the hydrophobic agent is not soluble in the aqueous carrier. In some embodiments, the hydrophobic agent has a solubility in water of less than 1 mg/mL.

In some embodiments, the hydrophobic agent is a therapeutic agent. In some embodiments, the therapeutic agent is a small molecule drug. In some embodiments, the therapeutic agent is a biomolecule. In some embodiments, the hydrophobic agent is a Biopharmaceutics Classification System (BCS) class II or class IV agent. In some embodiments, the therapeutic agent has a narrow therapeutic index. In some embodiments, the therapeutic agent a high-potency drug.

In some embodiments, the hydrophobic agent is an excipient or an adjuvant.

In some embodiments, the concentration of the hydrophobic agent in the suspension is about 5 mg/mL to about 1,000 mg/mL. In some embodiments, the amount of the hydrophobic agent is less than 0.1 mg.

In other aspects, provided herein is an ASD forming solution comprising: an organic carrier, a hydrophobic agent, and a polymer crystallization inhibitor, wherein the polymer inhibits crystallization of the agent in a dried state.

In some embodiments, the ASD forming solution has a viscosity of 0.5 cP to 3 cP. In some embodiments, the ASD forming solution has a viscosity of between 1 cP to 2 cP. In some embodiments, the ASD forming solution has a viscosity of 1.2 cP to about 1.8 cP.

In some embodiments, the ASD forming solution comprises a viscosity modulating agent. In some embodiments, the viscosity-modulating agent is a viscosity-enhancing agent. In some embodiments, the viscosity-enhancing agent is ethylene glycol, polyethylene glycol, glycerol, or propylene glycerol, or any combination thereof. In some embodiments, the viscosity-modulating agent is a viscosity-reducing agent. In some embodiments, the viscosity-reducing agent is a salt or an amino acid, or a combination thereof. In some embodiments, the concentration of the viscosity-modulating agent in the ASD forming solution is 1% to 40%. In some embodiments, the viscosity-modulating agent has a ratio to the organic carrier of 1:100 to 40:100 by volume. In some embodiments, the amount of viscosity-modulating agent in the mixture is based on a desired viscosity of the mixture.

In some embodiments, the organic carrier of the ASD forming solution comprises a component selected from the group consisting of acetone, dimethylformamide, ethanol, methanol, pentane, hexane, heptane, ethyl acetate, isopropyl alcohol, ethylene glycol, and dimethyl sulfoxide. In some embodiments, the ASD forming solution comprises at least 30% organic carrier by volume.

In some embodiments, the polymer is selected based on one or more characteristics of the hydrophobic agent.

In some embodiments, the polymer is selected from the group consisting of a cellulose derivative, poly(vinyl pyrrolidinone) (PVP), poly(vinyl pyrrolidinone-co-vinyl acetate) (copovidone, or PVPVA), hydroxypropyl methyl cellulose (hypromellose, or HPMC), and HPMC acetate succinate (hypromellose acetate succinate or HPMC-AS), and eudragit, or any combination thereof. In some embodiments, the polymer is a grade selected from the group consisting of LF, MF, HF, LMP, MMP, HMP, LG, MG, and HG. In some embodiments, the polymer is HPMC-AS-LF. In some embodiments, the ratio of the concentration of the polymer to the concentration of the drug in the ASD forming solution is from 1:20 to 20:1. In some embodiments, the ratio of the concentration of the polymer to the concentration of the drug in the ASD forming solution is 1:2 to 2:1.

In other aspects, provided herein is a composition comprising a dried or lyophilized suspension, wherein, upon reconstitution, any suspension described herein is formed.

In other aspects, provided herein is a dosage form prepared by any method described herein. In some embodiments, the dosage form comprises a fixed amount of the hydrophobic agent, wherein the hydrophobic agent is a therapeutic agent, and a fixed amount of a second hydrophobic agent. In some embodiments, the second hydrophobic agent is a therapeutic agent. In some embodiments, the second hydrophobic agent is a pharmaceutically acceptable excipient.

In other aspects, provided herein is a plurality of the dosage forms described herein prepared as a batch of drug dosage forms, wherein the amount of the hydrophobic agent of each drug dosage form of the batch does not vary more than 5% from the average.

In other aspects, provided herein is a system for producing a dosage form, said system comprising: a suspension comprising a hydrophobic agent, wherein the suspension comprises an aqueous carrier and a plurality of nanoparticles, wherein each nanoparticle of the plurality of nanoparticles comprises the hydrophobic agent, and wherein the viscosity of the suspension is less than 2 cP; and a high frequency droplet dispenser comprising about 100 to about 1000 individual nozzles, each nozzle configured to deposit at least one suspension droplet onto a substrate of the dosage form.

In some embodiments, each nozzle has an aperture diameter of about 20 μm to about 120 μm.

In some embodiments, the suspension droplet is deposited onto the substrate using a thermal process. In some embodiments, the suspension droplet is deposited onto the substrate using a piezoelectric process.

In other aspects, provided herein is a system for producing a dosage form, said system comprising: an amorphous solid dispersion (ASD) comprising a hydrophobic agent, the method comprising depositing droplets of an ASD forming solution comprising the hydrophobic agent, an organic carrier, and a polymer onto a substrate using a droplet dispenser, wherein the ratio of the polymer and the hydrophobic agent inhibits crystallization of the hydrophobic agent in a dried state on the substrate, and wherein the viscosity of the ASD forming solution is less than 2 cP; and a high frequency droplet dispenser comprising about 100 to about 1000 individual nozzles, each nozzle configured to deposit at least one solution droplet onto a substrate of the dosage form. In some embodiments, each nozzle has an aperture diameter of about 20 μm to about 120 μm. In some embodiments, the solution droplet is deposited onto the substrate using a thermal process. In some embodiments, the solution droplet is deposited onto the substrate using a piezoelectric process.

In other aspects, provided herein is a dispensing cartridge comprising the ASD forming solution as described herein.

In other aspects, provided herein is a dosage form comprising a nanosuspension of a hydrophobic agent deposited on a substrate via droplets.

In other aspects, provided herein is a dosage form comprising an amorphous solid dispersion of a hydrophobic agent deposited on a substrate via droplets.

In other aspects, provided herein is a method of determining a ratio of a hydrophobic agent and a polymer that forms an amorphous solid dispersion, the method comprising dispensing onto a substrate an amount of a first precursor solution comprising a first organic carrier, the hydrophobic agent, and a first viscosity-enhancing agent, and an amount of a second precursor solution comprising a second organic carrier, a polymer, and a second viscosity-enhancing agent to determine the ratio of the hydrophobic agent and the polymer that forms an amorphous solid dispersion of the hydrophobic agent and the polymer, wherein the dispensing causes the first precursor solution to contact the second precursor solution. In some embodiments, the dispensing mixes the first precursor solution and the second precursor solution on the substrate. In some embodiments, the dispensing mixes the first precursor solution and the second precursor solution prior to being applied to the substrate. In some embodiments, the amount of the first precursor solution and the amount of the second precursor solution are dispensed such that a gradient of ratios of the agent and the polymer are formed in an area on the substrate. In some embodiments, the amount of the first precursor solution and the amount of the second precursor solution are dispensed such that an area on the substrate has a pre-determined ratio of the agent and the polymer. In some embodiments, the amount of the first precursor solution is dispensed from a first sub-nanoliter droplet dispenser, and wherein the amount of the second precursor solution is dispensed from a second sub-nanoliter droplet dispenser. In some embodiments, the method further comprises assessing the crystallization of the agent. In some embodiments, the first organic carrier and the second organic carrier are the same. In some embodiments, the first viscosity-enhancing agent and the second viscosity-enhancing agent are the same. In some embodiments, the substrate is a thin film.

In other aspects, provided herein is a droplet dispenser for producing a dosage form comprising a nanosuspension of a hydrophobic agent, the dispenser comprising: (a) a dispensing head comprising one or more nozzles; (b) a first cartridge containing a suspension comprising: an aqueous carrier; and a plurality of nanoparticles comprising a hydrophobic agent suspended in the aqueous carrier, wherein the viscosity of the suspension is less than 3 cP, wherein the first cartridge is operably coupled to the first dispensing head; and (c) a control system configured to dispense the mixture via the first dispensing head according to a dispensing pattern.

In some embodiments, the first dispensing head comprises about 100 to about 1000 nozzles. In some embodiments, each nozzle has an aperture diameter of about 20 μm to about 120 μm. In some embodiments, the first dispensing head dispenses droplets using a thermal process. In some embodiments, the first dispensing head dispenses droplets using a piezoelectric process. In some embodiments, the method further comprises a second dispensing head operably coupled to a second cartridge, wherein the second cartridge contains a second agent. In some embodiments, the method further comprises a third dispensing head operably coupled to a third cartridge, wherein the third cartridge contains a protective coating agent. In some embodiments, the method further comprises a fourth dispensing head operably coupled to a fourth cartridge, wherein the fourth cartridge contains an edible agent.

In other aspects, provided herein is a droplet dispenser for producing a dosage form comprising an amorphous solid dispersion of a hydrophobic agent, the dispenser comprising: (a) a first dispensing head comprising one or more nozzles; (b) a first mixture cartridge containing an ASD forming solution comprising an organic carrier, the agent, a polymer, and a viscosity-modulating agent, wherein the agent and the polymer are present in the mixture in a ratio such that crystallization of the agent is inhibited in a dried state on a substrate, wherein the first mixture cartridge is operably coupled to the first dispensing head; and (c) a control system configured to dispense the mixture via the dispensing head according to dispensing pattern.

In some embodiments, the first dispensing head comprises about 100 to about 1000 nozzles. In some embodiments, each nozzle has an aperture diameter of about 20 μm to about 120 μm. In some embodiments, the first dispensing head dispenses droplets using a thermal process. In some embodiments, the first dispensing head dispenses droplets using a piezoelectric process. In some embodiments, the method further comprises a second dispensing head operably coupled to a second mixture cartridge, wherein the second mixture cartridge contains a second mixture comprising a second agent. In some embodiments, the method further comprises a third dispensing head operably coupled to a third mixture cartridge, wherein the third mixture cartridge contains a protective coating mixture. In some embodiments, the method further comprises a fourth dispensing head operably coupled to a fourth mixture cartridge, wherein the fourth mixture cartridge contains an edible mixture.

In other aspects, provided herein is a gradient of two components on a substrate, comprising a first component comprising a hydrophobic agent; a second component comprising a polymeric crystallization inhibitor; and a ratio of the first component to the second component, wherein the ratio changes across the gradient.

In some embodiments, the first component further comprises an organic carrier. In some embodiments, the first component further comprises a viscosity-enhancing agent. In some embodiments, the second component further comprises an organic carrier. In some embodiments, the second component further comprises a viscosity-enhancing agent. In some embodiments, the second component is capable of inhibiting crystallization of the hydrophobic agent. In some embodiments, the first and second components each comprise an organic carrier, wherein the organic carriers are the same. In some embodiments, the first and second components each comprise a viscosity-enhancing agent, wherein the viscosity-enhancing agents are the same. In some embodiments, the ratio becomes low enough at a certain point on the gradient to prevent crystallization of the hydrophobic agent. In some embodiments, the first and second components are dispensed onto the substrate from a sub-nanoliter droplet dispenser. In some embodiments, the first component is dispensed onto the substrate from a first sub-nanoliter droplet dispenser and the second component is dispensed onto the substrate from a second sub-nanoliter droplet dispenser. In some embodiments, the ratio changes at a consistent rate across the gradient.

### BRIEF DESCRIPTION OF THE DRAWINGS

FIG. 1 shows a microscopy image of a nano-suspension.

FIG. 2 shows a microscopy image of a dispensed nano-suspension on a substrate.

FIG. 3 shows a microscopy image of a dispensed nano-suspension on a substrate.

FIGS. 4A-4C each show a microscopy image of a dispensed nano-suspension on a substrate exposed to wetting by an advancing water line.

FIG. 5 shows a microscopy image of reconstituted nano-suspension obtained from droplets of nano-suspension deposited on a substrate. The resulting reconstituted nano-suspension was recovered and analyzed by optical microscopy, showing a homogeneous, well-dispersed suspension with no significant aggregation.

FIGS. 6A and 6B show schematics of depositions comprising a hydrophobic agent load and/or a polymer. FIG. 6A shows a schematic of a gradient deposition having varying amounts of the hydrophobic agent and/or polymer. FIG. 6B shows a schematic of areas having a deposition having with a certain amount of a hydrophobic agent relative to a polymer.

FIGS. 7A and 7B show schematics of deposition of a fast-crystallizing drug model, griseofulvin, and a polymer, HPMCAS-LF. FIG. 7A additionally shows the crystalline drug form when a deposition is produced without a polymer, and the amorphous polymer at 100% polymer load. FIG. 7B additionally shows the deposition of a 20% drug load resulting in an amorphous solid dispersion lacking a crystalline form of the drug.

### DETAILED DESCRIPTION

Provided herein, in certain aspects, are methods of making a dosage form comprising dispensing a plurality of droplets, such as in a pattern or patterned layer, of a formulation comprising a hydrophobic agent onto a substrate via a droplet dispenser, such as a high frequency droplet dispenser or a high frequency, sub-nanoliter droplet dispenser. In other aspects, provided herein are embodiments, such as composition, devices, and systems, useful with or produced from the methods described herein. In some embodiments, the methods described herein can be used to prepare formulations such as nanosuspensions and ASDs in a precise way (e.g., in personalized medicines), which can help improve the absorption and bioavailability of drugs.

Provided herein, in certain aspects, are methods of making a dosage form comprising dispensing a plurality of droplets, such as in a pattern or patterned layer, of a nano-suspension comprising a plurality of nanoparticles comprising a hydrophobic agent onto a substrate via a droplet dispenser, such as a high frequency droplet dispenser or a high frequency, sub-nanoliter droplet dispenser. In other aspects, provided herein are embodiments, such as composition, devices, and systems, useful with or produced from the methods described herein.

The disclosure of the present application is based on the unique perspectives and findings regarding the deposition of droplets of nano-suspensions comprising nanoparticles of a hydrophobic agent using a droplet dispenser (such as a high frequency droplet dispenser or high frequency, sub-nanoliter droplet dispenser), and associated production of dosage forms. As described herein, the findings demonstrate that precise and reproducible quantities of hydrophobic agent-containing nanoparticles in a nano-suspension can be deposited onto a substrate, such as a thin film, across a wide range of concentrations with no impact to nanoparticle size or chemical and physical stability during the process. Such deposition can be, e.g., performed using a droplet dispenser that deposits droplets using a thermal and/or piezoelectric process. Importantly, after the deposited droplets dry, the hydrophobic agent nanoparticles remain stable and can be successfully reconstituted by the addition of water. The compatibility of using nano-suspension formulations as a suitable “ink” enables such deposition techniques to be used as a general approach for poorly soluble drug molecules, significantly expanding the utility of the embodiments described herein. In some embodiments, the resulting material consists of a substrate, such as a thin film, comprising ultra-low microgram quantities of nanoparticles (such as drug nanoparticles), making this suitable for the preparation of products with low dosages that are challenging for traditional solid dosage forms and has implications for products for highly potent molecules or pediatric formulations. In addition, in certain embodiments, the final deposited product enables rapid dissolution and release of the hydrophobic agent (such as a drug), enabling fast absorption and precise delivery. Such deposition also allows for the custom tailoring of individual dosage forms on demand through control of the initial concentration in the formulation as well as adjustment of the deposition of material to form the dosage form. Moreover, the results described herein demonstrate that this approach can be undertaken with common commercially available off the shelf equipment with little custom modification.

Thus, in some aspects, provided is a method of making a dosage form comprising a hydrophobic agent, the method comprising: depositing a plurality of droplets of a nano-suspension onto a substrate using a high frequency droplet dispenser to form a dosage form, wherein said nano-suspension comprises: an aqueous carrier, and a plurality of nanoparticles suspended in said aqueous carrier, wherein said nanoparticles comprise the hydrophobic agent, and wherein the viscosity of the nano-suspension is less than 2 cP.

In other aspects, provided is a nano-suspension comprising: an aqueous carrier; a plurality of nanoparticles comprising a hydrophobic agent suspended in the aqueous carrier, wherein the viscosity of the nano-suspension is less than 2 cP.

In other aspects, provided is a composition comprising a dried or lyophilized nano-suspension, wherein upon reconstitution, a nano-suspension described herein is formed.

In other aspects, a plurality of a dosage form described herein is prepared as a batch of drug dosage forms, wherein the amount of the hydrophobic agent of each drug dosage form of the batch does not vary more than 5% from the average.

In other aspects, provided herein is a system for producing a dosage form, said system comprising: a nano-suspension comprising a hydrophobic agent, wherein the nano-suspension comprises an aqueous carrier and a plurality of nanoparticles, wherein each nanoparticle of the plurality of nanoparticles comprises the hydrophobic agent, and wherein the viscosity of the nano-suspension is less than 2 cP; and a high frequency droplet dispenser comprising about 100 to about 1000 individual nozzles, each nozzle configured to deposit at least one nano-suspension droplet onto a substrate of the dosage form.

Provided herein, in certain aspects, are method of making a dosage form comprising an amorphous solid dispersion (ASD), the method comprising depositing droplets of an ASD forming solution onto a substrate using a droplet dispenser. In other aspects, further provided are methods of identifying an ASD forming solution comprising an agent susceptible to crystallization, as well as embodiments encompassed by or produced using the methods described herein, e.g., ASD forming solutions and/or precursor solutions thereof, drug dosage forms, devices, and systems.

The disclosure of the present application is based on the inventor's unique perspective and finding regarding low-volume dispensing and deposition of droplets that form an ASD using droplet dispensers. Such low-volume dispensing and deposition greatly reduces the amount of an agent susceptible to crystallization, such as a hydrophobic agent, needed to identify and/or produce an ASD. In contrast, current methodology for studying ASDs involves spray drying techniques, including micro spray drying, which requires at least about 100 mg of initial loading material. The techniques taught herein can identify and produce ASDs from starting materials of microgram quantities or less. Moreover, as taught herein, two or more precursor solutions can be dispensed from two or more droplet dispensers, thus enabling gradient deposition studies to identifying ratios of an agent susceptible to crystallization and one or more polymers that provide an ASD of the agent. Involved with the discoveries detailed herein is the finding that solutions comprising an organic carrier can be formed such that they are compatible for use with droplet dispensers, such as a sub-nanoliter droplet dispenser.

Thus, in some aspects, provided is a method of making a dosage form comprising an amorphous solid dispersion (ASD) comprising a hydrophobic agent, the method comprising depositing droplets of an ASD forming solution comprising the hydrophobic agent, an organic carrier, and a polymer onto a substrate using a droplet dispenser, wherein the ratio of the polymer and the hydrophobic agent inhibits crystallization of the hydrophobic agent in a dried state on the substrate.

In other aspects, provided herein is an ASD forming solution comprising: an organic carrier, a hydrophobic agent, a polymer, and a viscosity-modulating agent, wherein the polymer inhibits crystallization of the agent in a dried state.

In other aspects, provided is a dispensing cartridge comprising an ASD forming solution, or a precursor solution, described herein.

In other aspects, provided is a dosage form comprising an amorphous solid dispersion of a hydrophobic agent deposited on a substrate via droplets.

In other aspects, provided is a dosage form prepared by a method described herein.

In other aspects, provided is a method of determining a ratio of a hydrophobic agent and a polymer that forms an amorphous solid dispersion, the method comprising dispensing onto a substrate an amount of a first mixture comprising a first organic carrier, the hydrophobic agent, and a first viscosity-enhancing agent, and an amount of a second mixture comprising a second organic carrier, a polymer, and a second viscosity-enhancing agent to determine the ratio of the hydrophobic agent and the polymer that forms an amorphous solid dispersion, wherein the dispensing is performed in such a manner that the first mixture and the second mixture are mixed on, or prior to being applied to, the substrate.

In other aspects, provided is a droplet dispenser for producing a dosage form comprising an amorphous solid dispersion of a hydrophobic agent, the dispenser comprising: (a) a dispensing head comprising one or more nozzles; (b) an ASD forming solution comprising an organic carrier, the agent, a polymer, and a viscosity-modulating agent, wherein the agent and the polymer are present in the mixture in a ratio such that crystallization of the agent is inhibited in a dried state on a substrate, wherein the mixture cartridge is operably coupled to the dispensing head; and (c) a control system configured to dispense the mixture via the dispensing head according to dispensing pattern.

In other aspects, provided is a gradient on a substrate, the gradient comprising: a first position, a second position, and a third position each comprising a mixture of an amount of a first deposit and an amount of a second deposit, wherein the first deposit comprises a hydrophobic agent, wherein the ratio of the amount of the first deposit to the amount of the second deposit in said first position is less than the ratio of the amount of the first deposit to the amount of the second deposit in said second position, and the ratio of the amount of the first deposit to the amount of the second deposit in said second position is less than the ratio of amount of the first deposit to the amount of the second deposit in said third position, wherein the interaction between the first deposit and the second deposit in the mixture at the first position, the second position, and the third position produces a measurable effect of the hydrophobic agent of the first deposit. In some embodiments, the difference in the ratios of the amount of the first deposit to the amount of the second deposit in each of the first position, second position and third position is the same.

### I. Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

The terms “polypeptide” and “protein,” as used herein, may be used interchangeably to refer to a polymer comprising amino acid residues, and are not limited to a minimum length. Such polymers may contain natural or non-natural amino acid residues, or combinations thereof, and include, but are not limited to, peptides, polypeptides, oligopeptides, dimers, trimers, and multimers of amino acid residues. Full-length polypeptides or proteins, and fragments thereof, are encompassed by this definition. The terms also include modified species thereof, e.g., post-translational modifications of one or more residues, for example, methylation, phosphorylation glycosylation, sialylation, or acetylation.

Throughout this disclosure, various aspects of the claimed subject matter are presented in a range format. It should be understood that the description in range format is merely for convenience and brevity and should not be construed as an inflexible limitation on the scope of the claimed subject matter. Accordingly, the description of a range should be considered to have specifically disclosed all the possible sub-ranges as well as individual numerical values within that range. For instance, where a range of values is provided, it is understood that each intervening value, to the tenth of the unit of the lower limit, unless the context clearly dictates otherwise, between the upper and lower limit of that range and any other stated or intervening value in that stated range, is encompassed within the disclosure, subject to any specifically excluded limit in the stated range. Where the stated range includes one or both of the limits, ranges excluding either or both of those included limits are also included in the disclosure. In some embodiments, two opposing and open-ended ranges are provided for a feature, and in such description it is envisioned that combinations of those two ranges are provided herein. For example, in some embodiments, it is described that a feature is greater than about 10 units, and it is described (such as in another sentence) that the feature is less than about 20 units, and thus, the range of about 10 units to about 20 units is described herein.

The term “about” as used herein refers to the usual error range for the respective value readily known in this technical field. Reference to “about” a value or parameter herein includes (and describes) variations that are directed to that value or parameter per se. For example, description referring to “about X” includes description of “X.”

As used herein, including in the appended claims, the singular forms “a,” “or,” and “the” include plural referents unless the context clearly dictates otherwise. For example, “a” or “an” means “at least one” or “one or more.” It is understood that aspects and variations described herein include embodiments “consisting” and/or “consisting essentially of” such aspects and variations.

As used herein, a “subject” or an “individual,” which are terms that are used interchangeably, is a mammal. In some embodiments, a “mammal” includes humans, non-human primates, domestic and farm animals, and zoo, sports, or pet animals, such as dogs, horses, rabbits, cattle, pigs, hamsters, gerbils, mice, ferrets, rats, cats, monkeys, etc. In some embodiments, the subject or individual is human.

Those skilled in the art will recognize that several embodiments are possible within the scope and spirit of the present disclosure. The following description illustrates the disclosure and, of course, should not be construed in any way as limiting the scope of the inventions described herein.

### II. Methods of Making a Dosage Form

In certain aspects, provided herein are methods of making a dosage form comprising a plurality of nanoparticles comprising a hydrophobic agent. As discussed herein, various aspects of the methods are described in a modular fashion in subsequence sections, and one will readily appreciate that the methods encompass the embodiments described therein. Such description in a modular fashion is not intended to limit the scope of the disclosure, and based on the teachings provided herein one of ordinary skill in the art will readily appreciate that certain modules can be integrated, at least in part. The section heading used herein are for organizational purposes only and are not to be construed as limiting the subject matter described.

In some embodiments, the methods comprises dispensing droplets, such as according to a pattern or patterned layer, of a nano-suspension comprising a plurality of nanoparticles comprising a hydrophobic agent onto a substrate via a droplet dispenser (such as a high frequency droplet dispenser or a high frequency, sub-nanoliter droplet dispenser). In some embodiments, the nano-suspension is sourced from a dispensing cartridge. In some embodiments, the method comprises conveying a nano-suspension to a droplet dispenser or a component thereof, such as a dispensing head thereof. In some embodiments, the viscosity of the nano-suspension is about 1 cP to about 5 cP, such as less than 2 cP, including about 1 cp to less than 2 cp or about 1.2 cP to about 1.8 cP.

In some embodiments, the method comprises conveying a nano-suspension comprising a plurality of nanoparticles comprising a hydrophobic agent suspended in an aqueous carrier to a droplet dispenser (such as a high frequency droplet dispenser or a high frequency, sub-nanoliter droplet dispenser), wherein the viscosity of the nano-suspension is less than 2 cP, such as about 1 cp to less then 2 cp or about 1.2 cP to about 1.8 cP; and depositing a plurality of droplets, such as according to a pattern or a patterned layer, of the nano-suspension onto a substrate using the droplet dispenser to form a dosage form.

In some embodiments, the droplet dispenser dispenses droplets in the direction of a substrate to deposit the droplets thereon. Generally speaking, the dispensed droplets from the droplet dispenser may experience changes in form during the deposition process, e.g., (a) one droplet may merge with another droplet after being dispensed from the droplet dispenser (whether in the air or on the substrate), and (b) deposited droplets dry on the substrate. Description of droplets is provided herein and may be applicable to the various states that occur during the deposition process. Such description is not intended to be limited to only a certain state unless otherwise expressly noted. In some embodiments, the depositing comprises dispensing a number of droplets of the nano-suspension per square inch (also referred to herein as dots per inch). In some embodiments, the resulting pattern or patterned layer produced by a method described herein comprises, at least to a degree, merging of one or more dispensed drops of a nano-suspension. Such a pattern of droplets need not be regular, and in some embodiments, reflects a number of droplets applied to a set area of a substrate. In some embodiments, the deposited droplets (whether or not merged prior to or upon the substrate) resemble a pattern on a macroscale, such as a shape. In some embodiments, the dosage form comprises one or more layers of droplets deposited on a substrate, such as formed from one or more passes of a droplet dispenser over an area of a dosage form. In some embodiments, the patterned layer, prior to drying, is a continuous layer of the nano-suspension, e.g., the deposited droplets are interconnected to at least a degree. In some embodiments, the droplets of a nano-suspension are deposited at about 1 dot per inch (dpi) to about 1,000 dpi, such as any of about 1 dpi to about 720 dpi, about 72 dpi to about 720 dpi, about 72 dpi to about 500 dpi, or about 72 dpi to about 300 dpi. In some embodiments, the droplets of a nano-suspension are deposited at about 1 dpi or greater, such as about any of 20 dpi or greater, 50 dpi or greater, 72 dpi or greater, 100 dpi or greater, 120 dpi or greater, 150 dpi or greater, 200 dpi or greater, 250 dpi or greater, 300 dpi or greater, 350 dpi or greater, 400 dpi or greater, 450 dpi or greater, 500 dpi or greater, 550 dpi or greater, 600 dpi or greater, 650 dpi or greater, 700 dpi or greater, 720 dpi or greater, 750 dpi or greater, 800 dpi or greater, 850 dpi or greater, 900 dpi or greater, 950 dpi or greater, or 1,000 dpi or greater. In some embodiments, the droplets of a nano-suspension are deposited at about 1 dot per inch (dpi) to about 1,000 dpi, such as any of about 1 dpi to about 720 dpi, about 72 dpi to about 720 dpi, about 72 dpi to about 500 dpi, or about 72 dpi to about 300 dpi. In some embodiments, the droplets of a nano-suspension are deposited at about 1 dpi or greater, such as about any of 20 dpi or greater, 50 dpi or greater, 72 dpi or greater, 100 dpi or greater, 120 dpi or greater, 150 dpi or greater, 200 dpi or greater, 250 dpi or greater, 300 dpi or greater, 350 dpi or greater, 400 dpi or greater, 450 dpi or greater, 500 dpi or greater, 550 dpi or greater, 600 dpi or greater, 650 dpi or greater, 700 dpi or greater, 720 dpi or greater, 750 dpi or greater, 800 dpi or greater, 850 dpi or greater, 900 dpi or greater, 950 dpi or greater, or 1,000 dpi or greater.

In some embodiments, the dispensed or deposited droplet of a nano-suspension has a diameter of about 1 nm to about 200 μm, such as any of about 20 nm to about 800 nm, about 100 nm to about 500 nm, or about 10 μm to about 100 μm. In some embodiments, the dispensed or deposited droplet of a nano-suspension has a diameter of at least about 1 nm, such as at least about any of 10 nm, 20 nm, 30 nm, 40 nm, 50 nm, 60 nm, 70 nm, 80 nm, 90 nm, 100 nm, 110 nm, 120 nm, 130 nm, 140 nm, 150 nm, 160 nm, 170 nm, 180 nm, 190 nm, 200 nm, 210 nm, 220 nm, 230 nm, 240 nm, 250 nm, 260 nm, 270 nm, 280 nm, 290 nm, 300 nm, 310 nm, 320 nm, 330 nm, 340 nm, 350 nm, 360 nm, 370 nm, 380 nm, 390 nm, 400 nm, 410 nm, 420 nm, 430 nm, 440 nm, 450 nm, 460 nm, 470 nm, 480 nm, 490 nm, 500 nm, 525 nm, 550 nm, 575 nm, 600 nm, 625 nm, 650 nm, 675 nm, 700 nm, 725 nm, 750 nm, 775 nm, 800 nm, 825 nm, 850 nm, 875 nm, 900 nm, 925 nm, 950 nm, 975 nm, 1 μm, 5 μm, 10 μm, 15 μm, 20 μm, 25 μm, 30 μm, 35 μm, 40 μm, 45 μm, 50 μm, 60 μm, 70 μm, 80 μm, 90 μm, or 100 μm. In some embodiments, the average of the population of dispensed or deposited droplets of a dosage form described herein meet the criteria discussed above.

In some embodiments, the dispensed or deposited droplets of a nano-suspension comprise an average volume of about 1 picoliter (pL) to about 1 nL, such as about 1 pL to about 500 pL or about 1 pL to about 50 pL. In some embodiments, the dispensed or deposited droplets of a nano-suspension comprises comprise an average volume of about 1 nL or less, such as about any of 950 pL or less, 900 pL or less, 850 pL or less, 800 pL or less, 750 pL or less, 700 pL or less, 650 pL or less, 600 pL or less, 550 pL or less, 500 pL or less, 450 pL or less, 400 pL or less, 350 pL or less, 300 pL or less, 250 pL or less, 200 pL or less, 150 pL or less, 100 pL or less, 75 pL or less, 50 pL or less, or 25 pL or less.

In some embodiments, the method further comprises coupling a cartridge containing a nano-suspension and a droplet dispenser (such as a high frequency droplet dispenser or high frequency sub-nanoliter droplet dispenser). In some embodiments, the method further comprises selecting a cartridge comprises a nano-suspension to produce a desired dosage form, such as based on one or more of a desired characteristics of the final dosage form or one or more patterned layers thereon, e.g., amount of the hydrophobic agent to be deposited on the dosage form, a characteristic a patterned layer (such as any of dpi, shape, size, and position on the dosage form), a release profile of a hydrophobic agent, and a feature of the final dosage form (e.g., structural integrity and/or protection of the patterned layer). In certain embodiments, the method comprises dispensing at least one nano-suspension and, optionally, another material (e.g., a food safe material to form a label), wherein the methods encompass selection of the each dispensed component. In some embodiments, the method comprises selecting a nano-suspension from a library of nano-suspension (such as compiled in a kit). In some embodiments, the method further comprises making the nano-suspension.

In some embodiments, the method comprises designing a pattern, such as characterized by dpi, of droplets of a nano-solution comprising a plurality of nanoparticles comprising a hydrophobic agent. In some embodiments, the method of designing comprises determining (such as calculating) one or more settings based on any one or more of: the total amount of the hydrophobic agent of the dosage form; the concentration of the hydrophobic agent in the nano-suspension; the desired surface area of exposure of the nano-suspension, or a dried form thereof, of the dosage form; the desired release profile of the hydrophobic agent; and a characteristic of the sub-nanoliter droplet dispenser (or use thereof), such as aperture diameter, ejection frequency, and produced drop size.

In some embodiments, the method comprises creating and/or receiving instructions to control the droplet dispenser, such as to form a dosage form described herein. In some embodiments, the instructions are personalized for an individual.

In some embodiments, the method further comprises performing a drying time following dispensing of a nano-suspension on a substrate, e.g., such that the nano-suspension can dry, at least to a degree, and/or the nano-suspension can associate with (such as be absorbed into) the substrate. In some embodiments, the method further comprises one or more processing steps following dispensing of a nano-suspension on a substrate, such as cutting a substrate into individual dosage forms and/or packing one or more dosage forms.

In some embodiments, the method further comprises cleaning one or more dispensing head of a droplet dispenser, such as by dispensing a non-drug containing solvent. For example, such cleaning steps help to ensure that a nano-suspension has not clogged a nozzle of a dispensing head and/or to reduce carry-over of a hydrophobic agent.

### III. Nano-Suspensions

Provided herein, in certain aspects, is a nano-suspension comprising a plurality of nanoparticles comprising a hydrophobic agent suitable for use in and/or with described embodiments herein including the methods, devices, dispensing cartridges, compositions, and systems disclosed herein. In some embodiments, the nano-suspension comprises a carrier (such as an aqueous carrier); and a plurality of nanoparticles comprising a hydrophobic agent suspended in the carrier, wherein the nano-suspension has a viscosity suitable for use in the described embodiments herein including methods of making a dosage form using a sub-nanoliter droplet dispenser. In some embodiments, the nano-suspension comprises a viscosity-modulating agent, such as a viscosity-enhancing agent. Without being bound by theory, nano-suspensions can stabilize low solubility drugs as nanoparticles that can be absorbed rapidly by a patient or subject.

In some embodiments, provided herein is a nano-suspension comprising a carrier (such as an aqueous carrier), a plurality of nanoparticles comprising a hydrophobic agent suspended in the aqueous carrier, and a stabilizing component. In some embodiments, the aqueous carrier comprises water (such as about 100% water). In some embodiments, the stabilizing component is a polymer (e.g., polyvinylpyrrolidone, PVP), a surfactant (e.g., sodium dodecyl sulfate, SDS), or a mixture thereof. In some embodiments, the polymer stabilizing component comprises any one or more of a polyvinylpyrrolidone (PVP) including any type and/or grade thereof, hydroxypropyl cellulose (HPC), hydroxypropylmethyl cellulose (HPMC), methylcellulose (MC), poloxamer (including any type and/or grade thereof), and polyethylene glycol (PEG, including any type and/or grade thereof). In some embodiments, the surfactant stabilizing component comprises any one or more of a polysorbate or Tween (including any type and/or grade thereof), Vit-E TPGS, lecithin, sodium alkyl sulfonate salt(s), and sodium cholate salt(s). In some embodiments, the concentration of the hydrophobic agent, such as contained in the plurality of nanoparticles, in the nano-suspension is about 20 mg/mL to about 200 mg/mL, such as about 100 mg/mL. In some embodiments, the viscosity is less than 5 cP, such as less than about any of 4, cP, 3 cP, or 2 cP, including about 1.2 cP to about 1.8 cP. In some embodiments, the nano-suspension comprises a viscosity-modulating agent, such as a viscosity-enhancing agent.

In some embodiments, provided herein is a nano-suspension comprising an aqueous carrier, a plurality of nanoparticles comprising a hydrophobic agent suspended in the aqueous carrier, and a stabilizing component, wherein the viscosity of the nano-suspension is less than 5 cP, such as less than about any of 4, cP, 3 cP, or 2 cP, including about 1 cp to less than 2 cp or about 1.2 cP to about 1.8 cP, and wherein the concentration of the hydrophobic agent, such as contained in the plurality of nanoparticles, in the nano-suspension is about 20 mg/mL to about 200 mg/mL, such as about 100 mg/mL. In some embodiments, the aqueous carrier comprises water (such as about 100% water). In some embodiments, the stabilizing component is a polymer (e.g., PVP), a surfactant (e.g., SDS), or a mixture thereof. In some embodiments, the nano-suspension comprises a viscosity-modulating agent, such as a viscosity-enhancing agent.

In some embodiments, provided herein is a nano-suspension comprising an aqueous carrier (water), a plurality of nanoparticles comprising a hydrophobic agent suspended in the aqueous carrier, and a stabilizing component comprising a polymer (e.g., PVP), a surfactant (e.g., SDS), or a mixture thereof, wherein the viscosity of the nano-suspension is less than 5 cP, such as less than about any of 4, cP, 3 cP, or 2 cP, including about 1 cp to less than 2 cp or about 1.2 cP to about 1.8 cP, and wherein the concentration of the hydrophobic agent, such as contained in the plurality of nanoparticles, in the nano-suspension is about 20 mg/mL to about 200 mg/mL, such as about 100 mg/mL. In some embodiments, the nano-suspension comprises a viscosity-modulating agent, such as a viscosity-enhancing agent.

In some embodiments, provided is a nano-suspension comprising: (a) an aqueous carrier, wherein the aqueous carrier comprises about 100% water (wt %), (b) a plurality of nanoparticles comprising a hydrophobic agent, such as naproxen, suspended in the aqueous carrier, and (c) PVP and SDS, wherein the ratio of the hydrophobic agent, PVP, and SDS is about 1:0.25:0.01 (100:25:1), wherein the concentration of the hydrophobic agent in the nano-suspension is about 100 mg/mL, and wherein the viscosity of the nano-suspension is between about 1 cP and about 3 cP, including about 2.1 cP. In some embodiments, the nano-suspension comprises a viscosity-modulating agent, such as a viscosity-enhancing agent.

In some embodiments, provided is a nano-suspension comprising: (a) an aqueous carrier, wherein the aqueous carrier comprises water, (b) a plurality of nanoparticles comprising a hydrophobic agent, such as naproxen, suspended in the aqueous carrier, (c) PVP and SDS, wherein the ratio of the hydrophobic agent, PVP, and SDS is about 1:0.25:0.01 (100:25:1), and (d) a viscosity-modulating agent, such as a viscosity-enhancing agent, e.g., ethylene glycol, wherein the concentration of the hydrophobic agent in the nano-suspension is about 10 mg/mL, and wherein the viscosity of the nano-suspension is between about 1 cP and about 3 cP, including about 1.5 cP.

In the following subsections, additional description of the various aspects of the nano-suspensions described herein is provided. Such description in a modular fashion is not intended to limit the scope of the disclosure, and based on the teachings provided herein one of ordinary skill in the art will readily appreciate that certain modules can be integrated, at least in part. The section heading used herein are for organizational purposes only and are not to be construed as limiting the subject matter described.

### A. Aqueous Carriers

In some embodiments, the nano-suspension is a colloidal suspension of hydrophobic agent-containing nanoparticles in an aqueous carrier. In some embodiments, the aqueous carrier does not solubilize the hydrophobic agent-containing nanoparticles contained therein. In some embodiments, the aqueous carrier is a substantially aqueous solution, e.g., a solution comprising at least about 50% v/v, such as at least about any of 55% v/v, 60% v/v, 65% v/v, 70% v/v, 75% v/v, 80% v/v, 85% v/v, 90% v/v, 95% v/v, or 100% v/v, of an aqueous medium, such as water. In some embodiments, the aqueous medium is water, including distilled water, double-distilled water, deionized water, purified water, ultrapure water, DNase- and/or RNase-free water, protease-free water, water suitable for injection, or pharmaceutical-grade water.

In some embodiments, the aqueous carrier of a nano-suspension comprises water and an organic solvent. In some embodiments, the ratio of water and an organic solvent in an aqueous carrier is between about 6:4 to about 9.9:1, such as any of 6:4, 7:3, 8:2, or 9:1. In some embodiments, the ratio of water and an organic solvent is greater than 10:1, greater than 100:1, greater than 1000:1, or greater than 10000:1. In some embodiments, the organic solvent is selected from the group consisting of acetonitrile, ethanol, isopropanol, DMSO, a polyethylene glycol (PEG), and a propylene glycol, or a mixture thereof. In some embodiments, the organic solvent is suitable for administration to an individual, such as a human. In some embodiments, the organic solvent at the amount that remains in a dosage form administered to an individual and produced from the nano-suspension described herein is suitable for administration to the individual.

### B. Viscosity

The nano-suspensions provided herein have a viscosity such that the nano-suspensions are suitable for use in the described embodiments herein, including methods of making a dosage form and droplet dispensers. In some embodiments, the viscosity of a nano-suspension provided herein is such that it can be printed with a sub-nanoliter droplet disperser. In some embodiments, the viscosity of a nano-suspension provided herein is such that it does not leak from a dispensing cartridge described herein.

In some embodiments, the viscosity of the nano-suspension is about 0.5 cP to about 5 cP, such as any of about 0.5 cP to about 2.5 cP, about 1 cP to about 2.5 cP, about 1.5 to about 2.5, or about 1.2 cP to about 1.8 cP. In some embodiments, the viscosity of the nano-suspension is less than about 5 cP, such as less than about any of 4.9 cP, 4.8 cP, 4.7 cP, 4.6 cP, 4.5 cP, 4.4 cP, 4.3 cP, 4.2 cP, 4.1 cP, 4.0 cP, 3.9 cP, 3.8 cP, 3.7 cP, 3.6 cP, 3.5 cP, 3.4 cP, 3.3 cP, 3.2 cP, 3.1 cP, 3.0 cP, 2.9 cP, 2.8 cP, 2.7 cP, 2.6 cP, 2.5 cP, 2.4 cP, 2.3 cP, 2.2 cP, 2.1 cP, 2.0 cP, 1.9 cP, 1.8 cP, 1.7 cP, 1.6 cP, 1.5 cP, 1.4 cP, 1.3 cP, 1.2 cP, 1.1 cP, 1.0 cP, 0.9 cP, 0.8 cP, 0.7 cP, 0.6 cP or 0.5 cP. In some embodiments, the viscosity of the nano-suspension is less than about 2 cP, such as less than about any of 1.95 cP, 1.9 cP, 1.85 cP, 1.8 cP, 1.75 cP, 1.7 cP, 1.65 cP, 1.6 cP, 1.55 cP, 1.5 cP, 1.45 cP, 1.4 cP, 1.35 cP, 1.3 cP, 1.25 cP, 1.2 cP, 1.15 cP, 1.1 cP, 1.05 cP, 1 cP, 0.95 cP, 0.9 cP, 0.85 cP, 0.8 cP, 0.75 cP, 0.7 cP, 0.65 cP, 0.6 cP, 0.55 cP, or 0.5 cP. In some embodiments, the viscosity of the nano-suspension is at least about 0.5 cP, such as at least about any of 0.6 cP, 0.7 cP, 0.8 cP, 0.9 cP, 1.0 cP, 1.1 cP, 1.2 cP, 1.3 cP, 1.4 cP, 1.5 cP, 1.6 cP, 1.7 cP, 1.8 cP, 1.9 cP, 2.0 cP, 2.1 cP, 2.2 cP, 2.3 cP, 2.4 cP, 2.5 cP, 2.6 cP, 2.7 cP, 2.8 cP, 2.9 cP, 3.0 cP, 3.1 cP, 3.2 cP, 3.3 cP, 3.4 cP, 3.5 cP, 3.6 cP, 3.7 cP, 3.8 cP, 3.9 cP, 4.0 cP, 4.1 cP, 4.2 cP, 4.3 cP, 4.4 cP, 4.5 cP, 4.6 cP, 4.7 cP, 4.8 cP, 4.9 cP, or 5.0 cP. In some embodiments, the viscosity of the nano-suspension is at least about 0.5 cP, such as at least about any of 0.55 cP, 0.6 cP, 0.65 cP, 0.7 cP, 0.75 cP, 0.8 cP, 0.85 cP, 0.9 cP, 0.95 cP, 1 cP, 1.05 cP, 1.1 cP, 1.15 cP, 1.2 cP, 1.25 cP, 1.3 cP, 1.35 cP, 1.4 cP, 1.45 cP, 1.5 cP, 1.55 cP, 1.6 cP, 1.65 cP, 1.7 cP, 1.75 cP, 1.8 cP, 1.85 cP, 1.9 cP, 1.95 cP, or 2.0 cP. In some embodiments, the viscosity of the nano-suspension is about any of 0.5 cP, 0.6 cP, 0.7 cP, 0.8 cP, 0.9 cP, 1.0 cP, 1.1 cP, 1.2 cP, 1.3 cP, 1.4 cP, 1.5 cP, 1.6 cP, 1.7 cP, 1.8 cP, 1.9 cP, 2.0 cP, 2.1 cP, 2.2 cP, 2.3 cP, 2.4 cP, 2.5 cP, 2.6 cP, 2.7 cP, 2.8 cP, 2.9 cP, 3.0 cP, 3.1 cP, 3.2 cP, 3.3 cP, 3.4 cP, 3.5 cP, 3.6 cP, 3.7 cP, 3.8 cP, 3.9 cP, 4.0 cP, 4.1 cP, 4.2 cP, 4.3 cP, 4.4 cP, 4.5 cP, 4.6 cP, 4.7 cP, 4.8 cP, 4.9 cP, or 5.0 cP. In some embodiments, the viscosity of the nano-suspension is about any of 0.5 cP, 0.55 cP, 0.6 cP, 0.65 cP, 0.7 cP, 0.75 cP, 0.8 cP, 0.85 cP, 0.9 cP, 0.95 cP, 1 cP, 1.05 cP, 1.1 cP, 1.15 cP, 1.2 cP, 1.25 cP, 1.3 cP, 1.35 cP, 1.4 cP, 1.45 cP, 1.5 cP, 1.55 cP, 1.6 cP, 1.65 cP, 1.7 cP, 1.75 cP, 1.8 cP, 1.85 cP, 1.9 cP, 1.95 cP, or 2.0 cP. In the embodiments described herein, the viscosity of the nano-suspension is assessed at about room temperature (such as about 20° C. to about 22° C., including at about any of 20° C., 21° C., or 22° C.).

Various components may, in some embodiments, contribute to the viscosity of a nano-suspension, including the concentration of a hydrophobic agent and nanoparticles thereof. In some embodiments, the nano-suspension further comprises a viscosity-modulating agent. In some embodiments, the viscosity-modulating agent is a viscosity-enhancing agent, e.g., increases the viscosity of a nano-suspension. In some embodiments, the viscosity-modulating agent is a viscosity-reducing agent, e.g., reduces the viscosity of a nano-suspension.

In some embodiments, the viscosity-enhancing agent is selected from the group consisting of ethylene glycol, polyethylene glycol, glycerol, propylene glycerol, and a cellulosic polymer, or mixtures thereof. In some embodiments, the cellulosic polymer is selected from a methylcellulose, hydroxymethyl cellulose, and hydroxypropylmethyl cellulose, or mixtures thereof.

In some embodiments, the viscosity-reducing agent is selected from the group consisting of a salt and an amino acid, or mixtures thereof.

In some embodiments, the viscosity-modulating agent, such as a viscosity-enhancing agent or a viscosity-reducing agent, has concentration in a nano-suspension of about 0.1% v/v to about 40% v/v, such as any of about 1% v/v to about 10% v/v, about 5% v/v to about 15% v/v, about 10% v/v to about 20% v/v, about 15% v/v to about 25% v/v, about 20% v/v to about 30% v/v, about 25% v/v to about 35% v/v, or about 30% v/v to about 40% v/v. In some embodiments, the viscosity-modulating agent, such as a viscosity-enhancing agent or a viscosity-reducing agent, has concentration in a nano-suspension of about 40% v/v or less, such as about any of 35% v/v, 30% v/v, 25% v/v, 20% v/v, 15% v/v, 10% v/v, 9% v/v, 8% v/v, 7% v/v, 6% v/v, 5% v/v, 4% v/v, 3% v/v, 2% v/v, 1% v/v, 0.9% v/v, 0.8% v/v, 0.7% v/v, 0.6% v/v, 0.5% v/v, 0.4% v/v, 0.3% v/v, 0.2% v/v, or 0.1% v/v. In some embodiments, the concentration of the viscosity-modulating agent, such as the viscosity-enhancing agent or a viscosity-reducing agent, in a nano-suspension is based on a desired viscosity of the nano-suspension.

In some embodiments, the ratio of the viscosity-modulating agent, such as a viscosity-enhancing agent or a viscosity-reducing agent, to the aqueous carrier of the nano-suspension is about 1:1000 to about 1:2, such as any of about 1:500 to about 1:2, about 1:100 to about 1:2, about 1:50 to about 1:2, about 1:20 to about 1:2, about 1:1000 to about 1:5, about 1:500 to about 1:5, about 1:100 to about 1:5, about 1:50 to about 1:5, about 1:20 to about 1:5, about 1:1000 to about 1:10, about 1:500 to about 1:10, about 1:100 to about 1:10, about 1:50 to about 1:10, or about 1:20 to about 1:10, or about 1:10 to about 1:5. In some embodiments, the ratio is about 1:5. In some embodiments, the ratio is about 1:10.

### D. Hydrophobic Agents and Nanoparticles Thereof

The nano-suspensions described herein comprise a hydrophobic agent in the form of a nanoparticle. In the method described herein, nano-suspensions are deposited onto a substrate using a droplet dispenser. In some embodiments, the droplet dispenser comprises a dispensing head comprising a nozzle having established aperture size characteristics. In some embodiments, the hydrophobic agent-containing nanoparticles of a nano-suspension have characteristics, such as size, that are based on an aperture size characteristic, such as diameter or largest crossing dimension.

In some embodiments, the nanoparticles of a nano-suspension, e.g., each nanoparticle of a plurality of nanoparticles, have a diameter (or largest crossing dimension) of about 500 μm or less, such as about any of 490 μm or less, 480 μm or less, 470 μm or less, 460 μm or less, 450 μm or less, 440 μm or less, 430 μm or less, 420 μm or less, 410 μm or less, 400 μm or less, 390 μm or less, 380 μm or less, 370 μm or less, 360 μm or less, 350 μm or less, 340 μm or less, 330 μm or less, 320 μm or less, 310 μm or less, 300 μm or less, 290 μm or less, 280 μm or less, 270 μm or less, 260 μm or less, 250 μm or less, 240 μm or less, 230 μm or less, 220 μm or less, 210 μm or less, 200 μm or less, 190 μm or less, 180 μm or less, 170 μm or less, 160 μm or less, 150 μm or less, 140 μm or less, 130 μm or less, 120 μm or less, 110 μm or less, 100 μm or less, 90 μm or less, 80 μm or less, 70 μm or less, 60 μm or less, 50 μm or less, 40 μm or less, 30 μm or less, 20 μm or less, 10 μm or less, 1 μm or less, 950 nm or less, 900 nm or less, 850 nm or less, 800 nm or less, 750 nm or less, 700 nm or less, 650 nm or less, 600 nm or less, 550 nm or less, 500 nm or less, 450 nm or less, 400 nm or less, 350 nm or less, 300 nm or less, 250 nm or less, 200 nm or less, 150 nm or less, 100 nm or less, or 50 nm or less.

In some embodiments, the nanoparticles of a nano-suspension have a D50 (also referred to as Dv(50) the median diameter (or largest crossing dimension) or the medium value of the particle size distribution) of about 250 μm to about 50 nm, such as any of 100 μm to about 1 μm, about 50 μm to about 500 nm, about 400 nm to about 100 nm, or about 250 nm to about 100 nm. In some embodiments, the nanoparticles of a nano-suspension have a D50 of about 250 μm or less, such as about any of 240 μm or less, 230 μm or less, 220 μm or less, 210 μm or less, 200 μm or less, 190 μm or less, 180 μm or less, 170 μm or less, 160 μm or less, 150 μm or less, 140 μm or less, 130 μm or less, 120 μm or less, 110 μm or less, 100 μm or less, 90 μm or less, 80 μm or less, 70 μm or less, 60 μm or less, 50 μm or less, 40 μm or less, 30 μm or less, 20 μm or less, 10 μm or less, 1 μm or less, 950 nm or less, 900 nm or less, 850 nm or less, 800 nm or less, 750 nm or less, 700 nm or less, 650 nm or less, 600 nm or less, 550 nm or less, 500 nm or less, 450 nm or less, 400 nm or less, 350 nm or less, 300 nm or less, 250 nm or less, 200 nm or less, 150 nm or less, 100 nm or less, or 50 nm or less.

In some embodiments, the nanoparticles of a nano-suspension have a D90 (also referred to as the Dv(90)) of about 250 μm to about 50 nm, such as any of 100 μm to about 1 μm, about 50 μm to about 500 nm, about 400 nm to about 100 nm, or about 250 nm to about 100 nm. In some embodiments, the nanoparticles of a nano-suspension have a D90 of about 250 μm or less, such as about any of 240 μm or less, 230 μm or less, 220 μm or less, 210 μm or less, 200 μm or less, 190 μm or less, 180 μm or less, 170 μm or less, 160 μm or less, 150 μm or less, 140 μm or less, 130 μm or less, 120 μm or less, 110 μm or less, 100 μm or less, 90 μm or less, 80 μm or less, 70 μm or less, 60 μm or less, 50 μm or less, 40 μm or less, 30 μm or less, 20 μm or less, 10 μm or less, 1 μm or less, 950 nm or less, 900 nm or less, 850 nm or less, 800 nm or less, 750 nm or less, 700 nm or less, 650 nm or less, 600 nm or less, 550 nm or less, 500 nm or less, 450 nm or less, 400 nm or less, 350 nm or less, 300 nm or less, 250 nm or less, 200 nm or less, 150 nm or less, 100 nm or less, or 50 nm or less.

In some embodiments, the diameter (or largest crossing dimension) of an aperture of a droplet dispenser is at least about 20% greater, such as at least about any of 25% greater, 30% greater, 35% greater, 40% greater, 45% greater, 50% greater, 55% greater, 60% greater, 65% greater, 70% greater, 75% greater, 80% greater, 85% greater, 90% greater, 95% greater, 100% greater, 125% greater, 150% greater, 175% greater, 200% greater, 225% greater, 250% greater, 275% greater, 300% greater, 350% greater, 400% greater, 450% greater, or 500% greater, than the diameter (or largest crossing dimension) of nanoparticles of a nano-suspension, e.g., the diameter (or largest crossing dimension) each nanoparticle of a plurality of nanoparticles.

There are numerous techniques known in the art for measuring particle characteristics, such as nanoparticle size. The description provided herein encompasses all techniques for evaluating particle characteristics of the nano-suspensions described herein. In some embodiments, the particle characteristic (such as size, e.g., diameter or largest crossing dimension) is measured using dynamic light scattering (DLS). In some embodiments, the particle characteristic (such as size, e.g., diameter or largest crossing dimension) is measured using disc centrifugation. In some embodiments, the particle characteristic (such as size, e.g., diameter or largest crossing dimension) is measured using nanoparticle tracking analysis. In some embodiments, the particle characteristic (such as size, e.g., diameter or largest crossing dimension) is measured using tunable resistive pule sensing. In some embodiments, the particle characteristic (such as size, e.g., diameter or largest crossing dimension) is measured using atomic force microscopy. In some embodiments, the particle characteristic (such as size, e.g., diameter or largest crossing dimension) is measured using electron microscopy.

In some embodiments, the hydrophobic agent is poorly water soluble. In some embodiments, the drug has a solubility in a solvent, e.g., water, of about 1 mg/mL or less, such about any of 750 μg/mL or less, 500 μg/mL or less, 250 μg/mL or less, 200 μg/mL or less, 150 μg/mL or less, 100 μg/mL or less, 50 μg/mL or less, 25 μg/mL or less, 1 μg/mL or less, 500 ng/mL or less, or 100 ng/mL. In some embodiments, the solvent is an aqueous solvent, such as water, a bicarbonate buffer, a biorelevant media, or a solvent comprising a surfactant. In some embodiments, hydrophobic agent is not soluble at a target concentration for the nano-suspension.

In some embodiments, the hydrophobic agent is sparingly soluble, slightly soluble, very slightly soluble, or practically insoluble (insoluble), as defined by the United States Pharmacopeia-National Formulary (USP-NF). In some embodiments, the hydrophobic agent is classified as sparingly soluble when having a solubility of between about 30 parts of solvent required for 1 part of solute to about 100 parts of solvent required for 1 part of solute. In some embodiments, the hydrophobic agent is classified as slightly soluble when having a solubility of between about 100 parts of solvent required for 1 part of solute to about 1,000 parts of solvent required for 1 part of solute. In some embodiments, the hydrophobic agent is classified as sparingly soluble when having a solubility of between about 1,000 parts of solvent required for 1 part of solute to about 10,000 parts of solvent required for 1 part of solute. In some embodiments, the hydrophobic agent is classified as sparingly soluble when having a solubility of about 10,000 or greater parts of solvent required for 1 part of solute. In some embodiments, the solvent is an aqueous solvent, such as water, a bicarbonate buffer, a biorelevant media, or a solvent comprising a surfactant.

In some embodiments, the hydrophobic agent is a Biopharmaceutics Classification System (BCS) class II drug. In some embodiments, the hydrophobic agent is a BCS class IV drug.

In some embodiments, the hydrophobic agent is a drug, such as a therapeutic agent useful for treating and/or preventing a condition.

In some embodiments, the hydrophobic agent comprises, or is, a small molecule drug. In some embodiments, the small molecule drug is a synthetic small molecule drug. In some embodiments, the small molecule drug has a molecular weight of about 1,500 Da or less, such as about any of 1,250 Da or less, 1,000 Da or less, 750 Da or less, or 500 Da or less. In some embodiments, the small molecule drug satisfies one or more of Lipinski's rule of five (no more than 5 hydrogen bond donors (the total number of nitrogen-hydrogen and oxygen-hydrogen bonds), no more than 10 hydrogen bond acceptors (all nitrogen or oxygen atoms), a molecular mass less than 500 daltons, and an octanol-water partition coefficient[10] (log P) that does not exceed 5).

In some embodiments, the hydrophobic agent comprises, or is, a biomolecule. In some embodiments, the biomolecule is a polypeptide, a lipid, or a nucleic acid. In some embodiments, the polypeptide is an antibody or a fragment thereof, including an antibody-drug-conjugate (ADC). In some embodiments, the polypeptide is a hormone. In some embodiments, the nucleic acid comprises an RNA or DNA, including pDNA, siRNA, miRNA, mRNA, and lnRNA.

In some embodiments, the hydrophobic agent has a narrow therapeutic index, e.g., a drug requiring accurate dosage amounts for safety purposes. In some embodiments, the hydrophobic agent has a broad therapeutic index. In some embodiments, the hydrophobic agent is a high-potency drug, e.g., a drug requiring low dosage amounts, such as less than about 1 mg.

In some embodiments, the hydrophobic agent is a regulatory approved compound, such as a drug approved for medical treatment by the United States Food and Drug Administration. In some embodiments, the hydrophobic agent is a prodrug. In some embodiments, the hydrophobic agent is a novel agent. In some embodiments, the hydrophobic agent is a fermentation product, semi-synthetic product, radiopharmaceutical, biological, biotechnology-derived product, herbal product, or crude products of animal or plant origin.

In some embodiments, the concentration of a hydrophobic agent in a nano-suspension described herein is about 1 μg/mL to about 1,000 mg/mL, such as any of about 0.1 mg/mL to about 1,000 mg/mL, about 1 mg/mL to about 500 mg/mL, about 5 mg/mL to about 1,000 mg/mL, or about 5 mg/mL to about 1,000 mg/mL. In some embodiments, the concentration of a hydrophobic agent in a nano-suspension described herein is about 1,000 mg/mL or less, such as about any of 750 mg/mL or less, 500 mg/mL or less, 250 mg/mL or less, 200 mg/mL or less, 150 mg/mL or less, 100 mg/mL or less, 50 mg/mL or less, 40 mg/mL or less, 30 mg/mL or less, 20 mg/mL or less, 10 mg/mL or less, 1 mg/mL or less, 900 μg/mL or less, 800 μg/mL or less, 700 μg/mL or less, 600 μg/mL or less, 500 μg/mL or less, 400 μg/mL or less, 300 μg/mL or less, 200 μg/mL or less, 100 μg/mL or less, 50 μg/mL or less, or 1 μg/mL or less. In some embodiments, the concentration of a hydrophobic agent in a nano-suspension described herein is at least about 1 μg/mL, such as at least about any of 50 μg/mL, 100 μg/mL, 200 μg/mL, 300 μg/mL, 400 μg/mL, 500 μg/mL, 600 μg/mL, 700 μg/mL, 800 μg/mL, 900 μg/mL, 1 mg/mL, 10 mg/mL, 20 mg/mL, 30 mg/mL, 40 mg/mL, 50 mg/mL, 100 mg/mL, 150 mg/mL, 200 mg/mL, 250 mg/mL, 500 mg/mL, 750 mg/mL or 1,000 mg/mL.

In some embodiments, the hydrophobic agent-containing nanoparticles of a nano-suspension described herein are stable for at least about any of 6 hours, 12 hours, 18 hours, 24 hours, 36 hours, 48 hours, or 72 hours. In some embodiments, the nano-suspension has a dispersity index of about 0.5 or less, such as about any of 0.4 or less, 0.3 or less, 0.2 or less, or 0.1 or less. In some embodiments, the nano-suspension has about 20% or less, such as any of 15% or less, 10% or less, 9% or less, 8% or less, 7% or less, 6% or less, 5% or less, 4% or less, 3% or less, 2% or less, or 1% or less, Ostwald ripening in about 12 hours. In some embodiments, the hydrophobic agent-containing nanoparticles of a nano-suspension have about 20% or less, such as any of 15% or less, 10% or less, 9% or less, 8% or less, 7% or less, 6% or less, 5% or less, 4% or less, 3% or less, 2% or less, or 1% or less, aggregation in about 12 hours.

In some embodiments, the hydrophobic agent-containing nanoparticles of a nano-suspension described herein are produced via a milling technique. In some embodiments, the milling technique is selected from the group consisting of a ball mill, wet media mill, dry media mill, jet mill (such as a fluidized bed jet mill), and a grinder. In some embodiments, the hydrophobic agent-containing nanoparticles of a nano-suspension described herein are produced via a hydrodynamic cavitation technique. In some embodiments, the hydrophobic agent-containing nanoparticles of a nano-suspension described herein are produced via an acoustic technique.

### C. Stabilizing Components

In some embodiments, the nano-suspension comprises a stabilizing component. In some embodiments, the stabilizing component is suitable for stabilizing the hydrophobic agent-containing nanoparticle in a nano-suspension described herein.

In some embodiments, the stabilizing component comprises any one or more of PVP, SDS, HPC, HPMC, a pluronic (poloxamer), Tween (polysorbate), or Vit-E TPGS. In some embodiments, the stabilizing component comprises PVP and/or SDS.

In some embodiments, the amount of a stabilizing component in a nano-suspension described herein is at least about 0.1% w/w, such as at least about any of 0.2% w/w, 0.3% w/w, 0.4% w/w, 0.5% w/w, 0.6% w/w, 0.7% w/w, 0.8% w/w, 0.9% w/w, 1.0% w/w, 1.5% w/w, 2.0% w/w, 2.5% w/w, 3.0% w/w, 3.5% w/w, 4.0% w/w, 4.5% w/w, 5.0% w/w, 6.0% w/w, 7.0% w/w, 8.0% w/w, 9.0% w/w, or 10% w/w. In some embodiments, the amount of a stabilizing component in a nano-suspension described herein is about 5% w/w or less, such as any of 4.5% w/w or less, 4.0% w/w or less, 3.5% w/w or less, 3.0% w/w or less, 2.5% w/w or less, 2.0% w/w or less, 1.5% w/w or less, 1.0% w/w or less, 0.9% w/w or less, 0.8% w/w or less, 0.7% w/w or less, 0.6% w/w or less, 0.5% w/w or less, 0.4% w/w or less, 0.3% w/w or less, 0.2% w/w or less, or 0.1% w/w or less.

In some embodiments, the amount of the stabilizing component in a nano-suspension described herein is based on a stabilizer component-to-drug weight ratio. In some embodiments, the stabilizer component-to-drug weight ratio is about 1:100 to about 2:1.

In some embodiments, the stabilizer component-to-drug weight ratio is at least about 1:100, such as at least about any of 2:100, 3:100, 4:100, 5:100, 6:100, 7:100, 8:100, 9:100, 10:100, 11:100, 12:100, 13:100, 14:100, 15:100, 16:100, 17:100, 18:100, 19:100, 20:100, 21:100, 22:100, 23:100, 24:100, 25:100, 30:100, 35:100, 40:100, 45:100, 50:100, 55:100, 60:100, 65:100, 70:100, 75:100, 80:100, 85:100, 90:100, 95:100, 1:1, 1.1:1, 1.2:1, 1.3:1, 1.4:1, 1.5:1, 1.6:1, 1.7:1. 1.8:1, 1.9:1, or 2:1.

In some embodiments, the amount of the stabilizing component in a nano-suspension described herein is based on a stabilizer component-to-carrier ratio. In some embodiments, the stabilizer component-to-carrier ratio is less than about 1:10, 1:50, 1:100, 1:200, 1:300, 1:400, 1:500, 1:600, 1:700, 1:800, 1:900, or 1:1000. In some embodiments, the stabilizer component-to-carrier ratio is greater than about 1:10,000, 1:1000, or 1:500. In some embodiments, the stabilizer component-to-carrier ratio is about 1:10, 1:50, 1:100, 1:200, 1:300, 1:400, 1:500, 1:600, 1:700, 1:800, 1:900, or 1:1000. In some embodiments, the stabilizer component-to-carrier ratio is from about 1:50 to about 1:500. In some embodiments, the stabilizer component-to-carrier ratio is about 1:50 or about 1:500.

### IV. Nano-Suspension Dispensing Cartridges and Composition Therein

Provided herein, in certain aspects, is a dispensing cartridge comprising (or suitable for use with) a nano-suspension described herein, or a derivative or precursor thereof. The dispensing cartridges described herein are generally suitable to hold one or more nano-suspensions described herein, such as in a reservoir of a dispensing cartridge, and are configured to interface with a dispensing head of a droplet dispenser such that a nano-suspension can be delivered to the dispensing head. In some embodiments, the dispensing cartridge is a disposable cartridge. In some embodiments, the dispensing cartridge is suitable for one-time or limited-time use.

In some embodiments, the dispensing cartridge comprises a material suitable for use with a nano-suspension described herein. In some embodiments, the dispensing cartridge comprises a material suitable for use in a pharmaceutical application, e.g., the material does not dissolve or leach a component thereof in the presence of a nano-suspension.

In some embodiments, the dispensing cartridge comprises one reservoir configured to hold a nano-suspension described herein. In some embodiments, the dispensing cartridge comprises more than one reservoir configured to hold a nano-suspension described herein. In embodiments of dispensing cartridges having more than one reservoir, in some instances a dispensing cartridge comprises any one or more of: (i) a first nano-suspension comprising a hydrophobic agent and a second nano-suspension comprising another agent, such as a hydrophobic agent; (ii) a first nano-suspension comprising a hydrophobic agent and a second nano-suspension comprising the hydrophobic agent, e.g., wherein the hydrophobic agent is at different concentrations in the first and second nano-suspensions and/or the first and second nano-suspensions comprise different non-drug components or concentrations thereof; and (iii) a first nano-suspension comprising a hydrophobic agent and a second solution comprising a material useful for an embodiments described herein, wherein the material is not a nano-suspension described herein. In some embodiments, provided is a dispensing cartridge comprising a material useful for an embodiment described herein, wherein the material is not a nano-suspension described herein. For example, in some embodiments, the dispensing cartridge comprises a fluid suitable for depositing on a substrate, such as to communicate a feature or identity (including unique identity) of a dosage form via a logo and/or label. In some embodiments, the label is a machine-readable label, such as a QR code or a barcode. Also disclosed herein are kits comprising one or more dispensing cartridges described herein.

In some embodiments, the dispensing cartridge comprises a reservoir, such as to contain a volume of a nano-suspension described herein. In some embodiments, the dispensing cartridge comprises a reservoir port, such as facilitate the flow of material(s) into or out of a reservoir. In some embodiments, the dispensing cartridge comprises a sponge material. In some embodiments, the sponge material is in a reservoir. In some embodiments, the dispensing cartridge comprises a dispensing head interface configured to enable the passage of a nano-suspension from a reservoir to a dispensing head of a droplet dispenser.

In some embodiments, provided herein is a composition comprising a form of a nano-suspension, including a derivative or precursor thereof. In some embodiments, the form of a nano-suspension is a frozen form, a dried form, or a lyophilized form of the nano-suspension. In some embodiments, the form of a nano-suspension can be processed to produce a nano-suspension described herein, such as via reconstitution. In some embodiments, the composition is contained in a dispensing cartridge.

### V. Nano-Suspension Dosage Forms

Provided herein, in certain aspects, is a dosage form comprising a patterned layer comprising a plurality of nanoparticles comprising a hydrophobic agent. In some embodiments, the dosage form comprises: a thin film substrate; and a patterned layer comprising a plurality of nanoparticles, wherein the nanoparticles comprise a hydrophobic agent.

In some embodiments, the dosage form is an oral dosage form (including an oral drug dosage form such as a capsule comprising a thin film substrate, e.g., a rolled and/or folded thin film substrate). In some embodiments, the dosage form is an intra-oral dosage form. In some embodiments, the dosage form is a buccal dosage form. In some embodiments, the dosage form is a sublingual dosage form. In some embodiments, the dosage form is a vaginal dosage form. In some embodiments, the dosage form is a rectal dosage form. In some embodiments, the dosage form is an ocular dosage from. In some embodiments, the dosage form comprises a component derived from deposition of a described nano-suspension using the methods provided herein. In some embodiments, the thin film substrate is suitable for human consumption. In some embodiments, the dosage form is suitable for contact-based delivery of a hydrophobic agent, such a transdermal dosage form. In some embodiments, the dosage form is an ocular dosage form. In some embodiments, the thin film substrate comprises a polymer (e.g., the thin film substrate is a polymer film). In some embodiments, the thin film substrate comprises one or more of chitosan, ethylenediaminetetraacetic acid, polyvinyl pyrrolidone, polyvinyl alcohol, alginate, agar, carrageenan, guar gum, xanthan gum, polycarbophil, and polyacrylic acid derivatives.

The dosage forms described herein may have a patterned layer of any shape and/or size. In some embodiments, characteristics of a patterned layer are based on an amount of a hydrophobic agent of a dosage form. For example, such characteristics of a patterned layer may be based on the hydrophobic agent concentration of a nano-suspension and the total desired amount of the hydrophobic agent to be deposited on a thin film substrate. In some embodiments, the characteristic of a patterned layer (such as any of shape, size, and position on the dosage form) are based on a desired release profile of a hydrophobic agent (e.g., a patterned layer may be designed to increase surface area to a bodily fluid) and/or feature of the final dosage form (e.g., structural integrity and/or protection of the patterned layer). In some embodiments, the term layer is not to be construed as to mean only a single deposition from a droplet dispenser described herein. In some embodiments, a patterned layer is formed from many instances of droplet deposition from a droplet dispenser described herein (e.g., repeated printing of an area using a nano-suspension described herein). For example, in producing a dosage form comprising a patterned layer, a droplet dispenser may be controlled to make one or more passes to deposit droplets of a nano-suspension to form the final patterned layer.

In some embodiments, the patterned layer is produced via a droplet dispenser depositing a nano-suspension at about 1 dot per inch (dpi) to about 1,000 dpi, such as any of about 1 dpi to about 720 dpi, about 72 dpi to about 720 dpi, about 72 dpi to about 500 dpi, or about 72 dpi to about 300 dpi. In some embodiments, the patterned layer is produced via a droplet dispenser depositing a nano-suspension at about 1 dpi or greater, such as about any of 20 dpi or greater, 50 dpi or greater, 72 dpi or greater, 100 dpi or greater, 120 dpi or greater, 150 dpi or greater, 200 dpi or greater, 250 dpi or greater, 300 dpi or greater, 350 dpi or greater, 400 dpi or greater, 450 dpi or greater, 500 dpi or greater, 550 dpi or greater, 600 dpi or greater, 650 dpi or greater, 700 dpi or greater, 720 dpi or greater, 750 dpi or greater, 800 dpi or greater, 850 dpi or greater, 900 dpi or greater, 950 dpi or greater, or 1,000 dpi or greater. In some embodiments, the patterned layer comprises a nano-suspension deposited at about 1 dot per inch (dpi) to about 1,000 dpi, such as any of about 1 dpi to about 720 dpi, about 72 dpi to about 720 dpi, about 72 dpi to about 500 dpi, or about 72 dpi to about 300 dpi. In some embodiments, the patterned layer comprises a nano-suspension deposited at about 1 dpi or greater, such as about any of 20 dpi or greater, 50 dpi or greater, 72 dpi or greater, 100 dpi or greater, 120 dpi or greater, 150 dpi or greater, 200 dpi or greater, 250 dpi or greater, 300 dpi or greater, 350 dpi or greater, 400 dpi or greater, 450 dpi or greater, 500 dpi or greater, 550 dpi or greater, 600 dpi or greater, 650 dpi or greater, 700 dpi or greater, 720 dpi or greater, 750 dpi or greater, 800 dpi or greater, 850 dpi or greater, 900 dpi or greater, 950 dpi or greater, or 1,000 dpi or greater.

In some embodiments, the deposited droplet of a nano-suspension has a diameter (or largest crossing dimension) of about 1 nm to about 200 μm, such as any of about 20 nm to about 800 nm, about 100 nm to about 500 nm, or about 10 μm to about 100 μm. In some embodiments, the deposited droplet of a nano-suspension has a diameter (or largest crossing dimension) of at least about 1 nm, such as at least about any of 10 nm, 20 nm, 30 nm, 40 nm, 50 nm, 60 nm, 70 nm, 80 nm, 90 nm, 100 nm, 110 nm, 120 nm, 130 nm, 140 nm, 150 nm, 160 nm, 170 nm, 180 nm, 190 nm, 200 nm, 210 nm, 220 nm, 230 nm, 240 nm, 250 nm, 260 nm, 270 nm, 280 nm, 290 nm, 300 nm, 310 nm, 320 nm, 330 nm, 340 nm, 350 nm, 360 nm, 370 nm, 380 nm, 390 nm, 400 nm, 410 nm, 420 nm, 430 nm, 440 nm, 450 nm, 460 nm, 470 nm, 480 nm, 490 nm, 500 nm, 525 nm, 550 nm, 575 nm, 600 nm, 625 nm, 650 nm, 675 nm, 700 nm, 725 nm, 750 nm, 775 nm, 800 nm, 825 nm, 850 nm, 875 nm, 900 nm, 925 nm, 950 nm, 975 nm, 1 μm, 5 μm, 10 μm, 15 μm, 20 μm, 25 μm, 30 μm, 35 μm, 40 μm, 45 μm, 50 μm, 60 μm, 70 μm, 80 μm, 90 μm, or 100 μm. In some embodiments, the average of the population of droplets (such as a patterned layer(s)) described herein meet the criteria discussed above.

In some embodiments, the patterned layer is, at least to a degree, within the thin film substrate (e.g., at least a portion of a nano-suspension described herein penetrates and/or is absorbed into the thin film substrate). In some embodiments, the patterned layer is, at least to a degree, on top of the thin film substrate (e.g., at least a portion of a nano-suspension described herein is at or above the surface of the thin film substrate). In some embodiments, the patterned layer is, at least to a degree, within the thin film substrate, and is, at least to a degree, on top of the thin film substrate.

In some embodiments, the dosage form comprises more than one patterned layer, e.g., a first patterned layer and a second patterned layer. In some embodiments, the first patterned layer comprises nanoparticles comprising a hydrophobic agent, and the second patterned layer comprises another agent. In some embodiments, the first patterned layer comprises nanoparticles comprising a hydrophobic agent, and the second patterned layer comprises nanoparticles comprising the hydrophobic agent. In some embodiments, the first patterned layer comprises nanoparticles comprising a hydrophobic agent, and the second patterned layer comprises nanoparticles comprising a second hydrophobic agent. In some embodiments, each patterned layer is produced via a layer-by-layer deposition technique.

In some embodiments, the dosage form comprises an amount of the hydrophobic agent of about 1 ng to about 1 mg, such as about 100 ng to about 500 ng, about 250 ng to about 750 ng, about 500 ng to about 1 pg, about 750 ng to about 1.25 pg, about 1 μg to about 2 pg, about 1 μg to about 10 pg, about 10 μg to about 50 pg, or about 25 μg to about 100 pg.

In some embodiments, the dosage form comprises an amount of the hydrophobic agent of at least about 1 ng, such as at least about any of 5 ng, 10 ng, 20 ng, 30 ng, 40 ng, 50 ng, 60 ng, 70 ng, 80 ng, 90 ng, 100 ng, 125 ng, 150 ng, 175 ng, 200 ng, 225 ng, 250 ng, 275 ng, 300 ng, 325 ng, 350 ng, 375 ng, 400 ng, 425 ng, 450 ng, 475 ng, 500 ng, 525 ng, 550 ng, 575 ng, 600 ng, 625 ng, 650 ng, 675 ng, 700 ng, 725 ng, 750 ng, 775 ng, 800 ng, 825 ng, 850 ng, 875 ng, 900 ng, 925 ng, 950 ng, 975 ng, 1 μg, 2 pg, 3 μg, 4 pg, 5 μg, 6 pg, 7 μg, 8 pg, 9 μg, 10 pg, 15 μg, 20 pg, 25 μg, 30 pg, 35 μg, 40 pg, 45 μg, 50 pg, 55 μg, 60 pg, 65 μg, 70 pg, 75 μg, 80 pg, 85 μg, 90 pg, 95 pg, or 100 pg.

In some embodiments, the dosage form further comprises a marking and/or a label. For example, in certain embodiments, the dosage form may contain printed information pertaining to any one or more of the active agent, amount of the active agent, the date produced, the individual the dosage form is produced for, the point of production, a trackable identification code, an expiration date, instructions for use, instructions for storage, or a company name and/or logo. In some embodiments, the marking and/or label is suitable for consumption, e.g., comprises a food safe dye.

In some embodiments, the dosage form comprises a protective coating, e.g., applied over a patterned layer. In some embodiments, the protective coating is applied to a dosage from via a droplet dispenser, such as a sub-nanoliter droplet dispenser or a high frequency, sub-nanoliter droplet dispenser. In some embodiments, the dosage form comprises a film coating.

In some embodiments, the dosage form is suitable for human use and/or consumption. In some embodiments, the dosage form is sterile.

In some embodiments, the dosage form is produced according to any of the methods described herein. For example, in some embodiments, provided is a dosage form produced by dispensing a patterned layer of a nano-suspension comprising a plurality of nanoparticles comprising a hydrophobic agent onto a substrate via a droplet dispenser (such as a sub-nanoliter droplet dispenser or a high frequency, sub-nanoliter droplet dispenser).

In certain aspects, provided here is a batch of dosage forms described herein. In some embodiments, the batch of dosage forms comprises at least 5 dosage forms, such as any of 10, 20, 30, 40, 50, 60, 70, 80, 90, or 100 dosage forms. In some embodiments, the batch of a drug dosage form described comprises individual dosage forms having an amount of a hydrophobic agent that does not vary more than 5% from the average.

### VI. Devices for Dispensing a Nano-Suspension Described Herein

Provided herein, in certain aspects, are devices for dispensing and depositing droplets of a nano-suspension described herein. In some embodiments, the device comprises a sub-nanoliter droplet dispenser. In some embodiments, the device comprises a high frequency, sub-nanoliter droplet dispenser. Sub-nanoliter droplet dispensers, including sub-nanoliter, high frequency droplet dispensers, are known in the art, including as described in U.S. Pat. Nos. 7,585,038, 7,963,635, and 6,851,786, which are hereby incorporated by reference in their entirety.

In some embodiments, provided is a droplet dispenser, such as a sub-nanoliter droplet dispenser or high frequency, sub-nanoliter droplet dispenser, for producing a dosage form, the dispenser comprising: (a) a dispensing head operably connected to a reservoir for a nano-suspension; and (b) a control system configured to control the deposition of the nano-suspension via the dispensing head. In some embodiments, provided is a droplet dispenser, such as a sub-nanoliter droplet dispenser or high frequency, sub-nanoliter droplet dispenser, for producing a dosage form, the dispenser comprising: (a) a dispensing head; (b) a nano-suspension dispensing cartridge receiving space, wherein the nano-suspension dispensing cartridge receiving space is configured to operably couple a nano-suspension dispensing cartridge to the dispensing head; and (c) a control system configured to control the deposition of a nano-suspension via the dispensing head according to a dispensing pattern. In some embodiments, provided is a droplet dispenser, such as a sub-nanoliter droplet dispenser or a high frequency, sub-nanoliter droplet dispenser, for producing a dosage form, the dispenser comprising: (a) a dispensing head; (b) a nano-suspension dispensing cartridge comprising a nano-suspension comprising a hydrophobic agent, wherein the nano-suspension dispensing cartridge is operably coupled to the dispensing head, wherein the nano-suspension comprises an aqueous carrier and a plurality of nanoparticles, wherein each nanoparticle of the plurality of nanoparticles comprises the hydrophobic agent; and (c) a control system configured to deposit the nano-suspension via the dispensing head according to a dispensing pattern. In some embodiments, the viscosity of the nano-suspension is less than 2 cP, such as between about 1 cp and less than 2 cp or 1.8 cP and about 1.2 cP.

In some embodiments, the dispensing head comprises about 10 to about 1000 individual nozzles. In some embodiments, the dispensing head comprises at least about 10, such as at least about any of 20, 30, 40, 50, 60, 70, 80, 90, 100, 150, 200, 250, 300, 350, 400, 450, 500, 550, 600, 650, 700, 750, 800, 850, 900, 950 or 1,000, individual nozzles.

In some embodiments, each nozzle of a dispensing head has an aperture (where the nano-suspension is dispensed from) with a largest cross-section (such as diameter) of about 20 μm to about 120 μm, such as about 20 μm to about 70 μm or about 30 μm to about 60 μm. In some embodiments, the nozzle of a dispensing head has an aperture with a largest cross-section (such as diameter) of at least about 20 μm, such as at least about any of 30 μm, 35 μm, 40 μm, 45 μm, 50 μm, 55 μm, 60 μm, 75 μm, 100 μm, or 120 μm.

In some embodiments, each nozzle of a dispensing head is configured to dispense a droplet of a nano-suspension having a volume of about 1 to about 500 picoliters (pL). In some embodiments, the nozzle of a dispensing head is configured to dispense a droplet of a nano-suspension having a volume of about 500 pL or less, such as about any of 450 pL or less, 400 pL or less, 350 pL or less, 300 pL or less, 250 pL or less, 200 pL or less, 150 pL or less, 100 pL or less, 75 pL or less, 50 pL or less, or 25 pL or less.

In some embodiments, the dispensing head is a continuous dispensing head. In some embodiments, the dispensing head is a drop-on-demand printing head. In some embodiments, the dispensing head is a thermal inkjet print head. In some embodiments, the dispensing head is a piezoelectric inkjet print head. In some embodiments, the dispensing head is a solenoid valve printing head. In some embodiments, the dispensing head is an electrostatic printing head. In some embodiments, the dispensing head is an acoustic printing head.

In some embodiments, the dispensing head is a sub-nanoliter dispensing head. In some embodiments, the dispensing head is a high frequency sub-nanoliter dispensing head (such as a dispensing head operating at an ejection frequency of above 2,000 Hz, such as above about 2.5 kHz, 3 kHz, 3.5 kHz, 4 kHz, 4.5 kHz, or 5 kHz).

In some embodiments, the dispensing device comprises more than one dispensing head. In some embodiments, the dispensing device further comprises a second dispensing head operably coupled to a second dispensing cartridge. In some embodiments, the dispensing device further comprises a third dispensing head operably coupled to a third dispensing cartridge. In some embodiments, the dispensing device further comprises a fourth dispensing head operably coupled to a fourth dispensing cartridge. In configurations comprising more than one dispensing head, the dispensing device is configured to be operably coupled to dispensing cartridges comprising any variety of materials, such as nano-suspensions, protective coatings, and label and/or marking materials (such as an edible ink).

### VII. Systems and Applications Comprising Nano-Suspension Embodiments Described Herein

Provided herein, in certain aspects, are systems and applications utilizing a method of making a dosage form described herein, and features associated therewith. In some embodiments, provided is a system comprises a dispensing device configured to receive instructions for printing a dosage form described herein. In some embodiments, the system is established at a point-of-care, such as in a hospital, care facility, pharmacy, or home. In some embodiments, the dosage form is a personalized dosage form, e.g., designed for an individual based on dosage form features such as the amount of a hydrophobic agent.

In some embodiments, the methods and dispensing devices described herein are suitable for personalized medicine. In some embodiments, the methods and dispensing devices described herein are suitable for point-of-care application. In some embodiments, the methods and dispensing devices described herein are suitable for rapid dosage form production (including on a small scale, such as less than 100 dosage forms). In some embodiments, the methods and dispensing devices described herein are suitable for producing dosage forms having a precise and accurate amount of a hydrophobic agent (such as required for hydrophobic agents having a narrow therapeutic index, high potency, and/or pediatric use). In some embodiments, the methods and dispensing devices described herein are suitable for producing dosage forms having a hydrophobic agent having poor stability (such as not stable for extended periods of time following production, e.g., greater than 24 hours). In some embodiments, the methods and dispensing devices described herein are suitable for non-pharmaceutical applications, such as nutraceutics.

### VIII. ASD Forming Solutions and Precursor Solutions Thereof

In certain aspects, provided herein are amorphous solid dispersion (ASD) forming solutions, and precursor solutions thereof. Such ASD forming solutions, and precursor solutions thereof, are useful for forming ASDs and/or studying and identifying conditions that result (or do not result) in the formation of an ASD comprising an agent susceptible to crystallization, such as a hydrophobic agent. As taught herein, the ASD forming solutions, including components and precursors thereof, may be dispensed and deposited in a number of ways. In some embodiments, the ASD forming solution is dispensed from a droplet dispenser in the form of a single solution. In some embodiments, the ASD forming solution is dispensed from two or more droplet dispensers, wherein the ASD forming solution forms following dispensing from the two or more droplet dispenser (e.g., precursor solutions from two or more droplet dispensers mix prior to and/or on a substrate to form the ASD forming solution and/or ASD). In some embodiments, the ASD forming solution is in the form of a single solution. In some embodiments, the precursor solutions described herein combine to form an ASD forming solution. For example, an ASD forming solution may be formed from two precursor solutions, such as a first precursor solution comprising at least one component (e.g., an agent susceptible to crystallization such as a hydrophobic agent) of the ASD forming solution and a second precursor solution comprising at least one component (e.g., a polymer) of the ASD forming solution. In some embodiments, the ASD forming solution is formulated such that, upon and/or following dispensing and/or depositing precursor solutions, the ADS forming solution produces an ASD comprising an agent that is susceptible to crystallization, such as a hydrophobic agent. In certain aspects described herein, one or more precursor solutions in a deposited condition (e.g., at a deposited ratio between an agent and a polymer) do not form an ASD. Without being bound by theory, ASDs convert crystalline drug material into amorphous material, which increases their solubility and thus enhances absorption.

In some aspects, the ASD forming solutions provided herein comprise an organic carrier, a polymer, and an agent susceptible to crystallization, such as a hydrophobic agent, and precursor solutions comprising at least one component of an ASD forming solution. As discussed herein, various aspects of the ASD forming solutions, and precursor solutions thereof, are described in a modular fashion in sections provided herein, and one will readily appreciate that the ASD forming solutions, and precursor solutions thereof, encompass the embodiments described therein and any combination thereof. Such description in a modular fashion is not intended to limit the scope of the disclosure, and based on the teachings provided herein one of ordinary skill in the art will readily appreciate that certain modules can be integrated, at least in part. The section heading used herein are for organizational purposes only and are not to be construed as limiting the subject matter described.

### A. Organic Carriers

In some aspects, provided herein are ASD forming solutions, or precursor solutions thereof, comprising an organic carrier. In some embodiments, the organic carrier improves solubility of an agent susceptible to crystallization, such as a hydrophobic agent, and/or improves carrier evaporation during the deposition process of a droplet dispenser. In some embodiments, the precursor solution does not comprise an organic carrier, e.g., a precursor solution comprising components compatible with an aqueous carrier.

In some embodiments, the organic carrier of the ASD forming solution comprises any one or more of acetone, dimethylformamide, ethanol, methanol, pentane, hexane, heptane, ethyl acetate, isopropyl alcohol, ethylene glycol, and dimethyl sulfoxide, or a combination thereof.

In some embodiments, the ASD forming solution, or a precursor solution thereof, comprises at least about 30% organic carrier by volume, such as at least about any of 35% organic carrier by volume, 40% organic carrier by volume, 45% organic carrier by volume, 50% organic carrier by volume, 55% organic carrier by volume, 60% organic carrier by volume, 65% organic carrier by volume, 70% organic carrier by volume, 75% organic carrier by volume, 80% organic carrier by volume, 85% organic carrier by volume, 90% organic carrier by volume, 95% organic carrier by volume, or 100% organic carrier by volume.

In some embodiments, the organic carrier comprises water. In some embodiments, the organic carrier comprises less than 30% water by volume, less than 20% water by volume, less than 10% water by volume, less than 5% water by volume, less than 2% water by volume, less than 1% water by volume, or less than 0.5% water by volume. In some embodiments, the organic carrier does not contain any water. In some embodiments, the organic carrier contains only trace amounts of water.

In some embodiments, the ASD forming solutions, or precursor solutions thereof, may comprise an aqueous carrier and a solubilizing agent. In such cases, the aqueous carrier replaces the organic carrier in the ASD forming solution or precursor solution thereof. An aqueous carrier could replace the organic carrier in any of the embodiments presented herein. In some embodiments, the solubilizing agent is selected from the group consisting of a surfactant, an emulsifier, a polyethylene glycol, propylene glycol, a polysorbate, a cyclodextrin, or a metal chelator (e.g., ethylenediamine-tetraacetic acid).

### B. Polymers

In some aspects, provided herein are ASD forming solutions, or precursor solutions thereof, comprising a polymer suitable for forming an ASD with an agent susceptible to crystallization, such as a hydrophobic agent. Generally speaking, the polymer at certain concentrations or ratios relative to an agent susceptible to crystallization inhibits formation of crystalline forms of the agent. In some embodiments, the precursor solution does not comprise a polymer, e.g., the polymer is contained in another precursor solution.

In some embodiments, the polymer of the ASD forming solution is suitable for formation of an amorphous solid dispersion (ASD) following deposition by a droplet dispenser. In some embodiments, the polymer modulates the viscosity of an ASD forming solution, or a precursor solution thereof.

In some embodiments, the polymer is selected based on one or more characteristics of an agent susceptible to crystallization, such as a hydrophobic agent. In some embodiments, the polymer is selected based on an interaction with an agent susceptible to crystallization, such as a hydrophobic agent. For example, in some embodiments, the interaction between the polymer and the agent includes one or more of van der Waals forces, ionic interactions, or hydrogen bonding.

In some embodiments, the polymer is selected from the group consisting of a cellulose derivative, poly(vinyl pyrrolidinone) (PVP), poly(vinyl pyrrolidinone-co-vinyl acetate) (copovidone, or PVPVA), hydroxypropyl methyl cellulose (hypromellose, or HPMC), and HPMC acetate succinate (hypromellose acetate succinate or HPMC-AS), and eudragit, or any combination thereof. In some embodiments, the polymer is a grade selected from the group consisting of LF, MF, HF, LMP, MMP, HMP, LG, MG, and HG.

In some embodiments, the polymer is HPMC-AS-LF.

In some embodiments, the weight ratio of the polymer to an agent susceptible to crystallization, such as a hydrophobic agent, in the ASD forming solution is from 1:20 to 20:1, such as 1:20 to any of 1:15, 1:10, 1:5, 1:2, 1:1, 2:1, 5:1, 10:1. 15:1 or 20:1. In some embodiments, the weight ratio is from 1:15 to any of 1:10, 1:5, 1:2, 1:1, 2:1, 5:1, 10:1. 15:1 or 20:1. In some embodiments, the weight ratio is from 1:10 to any of 1:5, 1:2, 1:1, 2:1, 5:1, 10:1. 15:1 or 20:1. In some embodiments, the weight ratio is from any of 1:5 to any of 1:2, 1:1, 2:1, 5:1, 10:1, 15:1, or 20:1. In some embodiments, the weight ratio is from 1:2 to any of 1:1, 2:1, 5:1, 10:1, 15:1, or 20:1. In some embodiments, the weight ratio is from 1:1 to any of 2:1, 5:1, 10:1, 15:1, or 20:1. In some embodiments, the weight ratio is from 2:1 to 5:1, 10:1, 15:1, or 20:1. In some embodiments, the weight ratio is from 5:1 to any of 10:1, 15:1, or 20:1. In some embodiments, the weight ratio is from 10:1 to 15:1 or 20:1. In some embodiments, the weight ratio is from 15:1 to 20:1.

In some embodiments, the weight ratio of the concentration of the polymer to the concentration of an agent susceptible to crystallization, such as a hydrophobic agent, in the ASD forming solution is 1:2 to 2:1.

In some embodiments, the polymer load of an ASD forming solution, or a precursor solution thereof, is about 1% to about 99% (w/w), such as any of about 5% to about 15%, about 15% to about 25%, about 25% to about 25%, about 35% to about 45%, about 45% to about 55%, about 55% to about 65%, about 65% to about 75%, about 75% to about 85%, or about 85% to about 95%. In some embodiments, the polymer load of an ASD forming solution, or a precursor solution thereof, is about any of 5%, 10%, 15%, 20%, 25%, 30%, 35%, 40%, 45%, 50%, 55%, 60%, 65%, 70%, 75%, 80%, 85%, 90%, or 95%.

### C. Agents Susceptible to Crystallization

The ASD forming solutions described herein comprise one or more agents susceptible to crystallization, such as a hydrophobic agent including a hydrophobic drug. In some embodiments, the agent is susceptible to crystallization during handling and/or production of a dosage form, e.g., spray drying and/or deposition via a droplet dispenser as described herein. In some embodiments, the agent is susceptible to crystallization at the desired concentrations in a final product, such as a drug dosage form, as described herein. Techniques for measuring crystallization are well known in the art, e.g., imaging, such as microscopy and polarized microscopy, raman spectroscopy, and differential scanning calorimetry. In some embodiments, crystallization is assessed at the time of producing the ASD (such as following deposition of an ASD forming solution according to the methods described herein). In some embodiments, crystallization is assessed following a period of time after producing the ASD, such as at any of about 1 days, 2 days, 3 days, 4 days, 5 days, 6 days, 1 week, 2 weeks, 3 weeks, 1 month, 3 months, 6 months, 9 months, or 1 year after producing the ASD. In some embodiments, the ASD is held for the period of time at a temperature above room temperature, such as at or above 37° C.

In some embodiments, the agent susceptible to crystallization is a hydrophobic agent. In some embodiments, the hydrophobic agent is poorly water soluble. In some embodiments, the hydrophobic agent has a solubility in a solvent, e.g., pure water, of about 1 mg/mL or less, such about any of 750 μg/mL or less, 500 μg/mL or less, 250 μg/mL or less, 200 μg/mL or less, 150 μg/mL or less, 100 μg/mL or less, 50 μg/mL or less, 25 μg/mL or less, 1 μg/mL or less, 500 ng/mL or less, or 100 ng/mL. In some embodiments, the solvent for assessing solubility is an aqueous solvent, such as water, a bicarbonate buffer, a biorelevant media, or a solvent comprising a surfactant. In some embodiments, hydrophobic agent is not soluble at a target concentration for the ASD forming solution.

In some embodiments, the hydrophobic agent is sparingly soluble, slightly soluble, very slightly soluble, or practically insoluble (insoluble), as defined by the United States Pharmacopeia-National Formulary (USP-NF). In some embodiments, the hydrophobic agent is classified as sparingly soluble when having a solubility of between about 30 parts of solvent required for 1 part of solute to about 100 parts of solvent required for 1 part of solute. In some embodiments, the hydrophobic agent is classified as slightly soluble when having a solubility of between about 100 parts of solvent required for 1 part of solute to about 1,000 parts of solvent required for 1 part of solute. In some embodiments, the hydrophobic agent is classified as sparingly soluble when having a solubility of between about 1,000 parts of solvent required for 1 part of solute to about 10,000 parts of solvent required for 1 part of solute. In some embodiments, the hydrophobic agent is classified as sparingly soluble when having a solubility of about 10,000 or greater parts of solvent required for 1 part of solute. In some embodiments, the solvent is an aqueous solvent, such as water, a bicarbonate buffer, a biorelevant media, or a solvent comprising a surfactant.

In some embodiments, the hydrophobic agent is a Biopharmaceutics Classification System (BCS) class II drug. In some embodiments, the hydrophobic agent is a BCS class IV drug.

In some embodiments, the hydrophobic agent is a drug, such as a therapeutic agent useful for treating and/or preventing a condition.

In some embodiments, the hydrophobic agent comprises, or is, a small molecule drug. In some embodiments, the small molecule drug is a synthetic small molecule drug. In some embodiments, the small molecule drug has a molecular weight of about 1,500 Da or less, such as about any of 1,250 Da or less, 1,000 Da or less, 750 Da or less, or 500 Da or less. In some embodiments, the small molecule drug satisfies one or more of Lipinski's rule of five (no more than 5 hydrogen bond donors (the total number of nitrogen-hydrogen and oxygen-hydrogen bonds), no more than 10 hydrogen bond acceptors (all nitrogen or oxygen atoms), a molecular mass less than 500 daltons, and an octanol-water partition coefficient[10] (log P) that does not exceed 5).

In some embodiments, the hydrophobic agent comprises, or is, a biomolecule. In some embodiments, the biomolecule is a polypeptide, a lipid, or a nucleic acid. In some embodiments, the polypeptide is an antibody or a fragment thereof, including an antibody-drug-conjugate (ADC). In some embodiments, the polypeptide is a hormone. In some embodiments, the nucleic acid comprises an RNA or DNA, including pDNA, siRNA, miRNA, mRNA, and lnRNA.

In some embodiments, the hydrophobic agent has a narrow therapeutic index, e.g., a drug requiring accurate dosage amounts for safety purposes. In some embodiments, the hydrophobic agent has a broad therapeutic index. In some embodiments, the hydrophobic agent is a high-potency drug, e.g., a drug requiring low dosage amounts, such as less than about 1 mg.

In some embodiments, the hydrophobic agent is a regulatory approved compound, such as a drug approved for medical treatment by the United States Food and Drug Administration. In some embodiments, the hydrophobic agent is a prodrug. In some embodiments, the hydrophobic agent is a novel agent. In some embodiments, the hydrophobic agent is a fermentation product, semi-synthetic product, radiopharmaceutical, biological, biotechnology-derived product, herbal product, or crude products of animal or plant origin.

In some embodiments, the concentration of a hydrophobic agent in an ASD forming solution, or a precursor thereof, described herein is about 1 μg/mL to about 1,000 mg/mL, such as any of about 0.1 mg/mL to about 1,000 mg/mL, about 1 mg/mL to about 500 mg/mL, about 5 mg/mL to about 1,000 mg/mL, or about 5 mg/mL to about 1,000 mg/mL. In some embodiments, the concentration of a hydrophobic agent in an ASD forming solution, or a precursor thereof, described herein is about 1,000 mg/mL or less, such as about any of 750 mg/mL or less, 500 mg/mL or less, 250 mg/mL or less, 200 mg/mL or less, 150 mg/mL or less, 100 mg/mL or less, 50 mg/mL or less, 40 mg/mL or less, 30 mg/mL or less, 20 mg/mL or less, 10 mg/mL or less, 1 mg/mL or less, 900 μg/mL or less, 800 μg/mL or less, 700 μg/mL or less, 600 μg/mL or less, 500 μg/mL or less, 400 μg/mL or less, 300 μg/mL or less, 200 μg/mL or less, 100 μg/mL or less, 50 μg/mL or less, or 1 μg/mL or less. In some embodiments, the concentration of a hydrophobic agent in an ASD forming solution, or a precursor solution thereof, described herein is at least about 1 μg/mL, such as at least about any of 50 μg/mL, 100 μg/mL, 200 μg/mL, 300 μg/mL, 400 μg/mL, 500 μg/mL, 600 μg/mL, 700 μg/mL, 800 μg/mL, 900 μg/mL, 1 mg/mL, 10 mg/mL, 20 mg/mL, 30 mg/mL, 40 mg/mL, 50 mg/mL, 100 mg/mL, 150 mg/mL, 200 mg/mL, 250 mg/mL, 500 mg/mL, 750 mg/mL or 1,000 mg/mL.

In some embodiments, the load of an agent susceptible to crystallization, such as a hydrophobic agent, of an ASD forming solution, or a precursor solution thereof, is about 1% to about 99% (w/w), such as any of about 5% to about 15%, about 15% to about 25%, about 25% to about 25%, about 35% to about 45%, about 45% to about 55%, about 55% to about 65%, about 65% to about 75%, about 75% to about 85%, or about 85% to about 95%. In some embodiments, the agent load of an ASD forming solution, or a precursor solution thereof, is about any of 5%, 10%, 15%, 20%, 25%, 30%, 35%, 40%, 45%, 50%, 55%, 60%, 65%, 70%, 75%, 80%, 85%, 90%, or 95%.

The ASD forming solutions described herein may prevent one or more agents susceptible to crystallization from crystallizing indefinitely or for a finite period of time. In some embodiments, the ASD forming solution forms an ASD wherein the agent susceptible to crystallization never crystallizes.

### D. Viscosity and Viscosity-Modulating Agents

The ASD forming solutions, and precursor solutions thereof, provided herein have a viscosity such that said solutions are suitable for use in the described embodiments herein, including methods of making a dosage form and droplet dispensers. In some embodiments, the viscosity of a solution provided herein (e.g., an ASD forming solution or a precursor solution thereof, such as a solution dispensed to from an ASD forming solution) is such that it can be dispensed with a droplet disperser, e.g., a sub-nanoliter droplet dispenser or a high frequency, sub-nanoliter droplet dispenser. In some embodiments, the viscosity of an ASD forming solution, or a precursor solution thereof, provided herein is such that it does not leak from a dispensing cartridge described herein or a droplet dispenser.

In some embodiments, the viscosity of an ASD forming solution, or a precursor solution thereof, is about 0.5 cP to about 5 cP, such as any of about 0.5 cP to about 3 cp, about 0.5 cP to about 2.5 cP, about 1 cP to about 2.5 cP, about 1.5 to about 2.5, about 1 cp to about 2 cp, or about 1.2 cP to about 1.8 cP. In some embodiments, the viscosity of an ASD forming solution, or a precursor solution thereof, is less than about 5 cP, such as less than about any of 4.9 cP, 4.8 cP, 4.7 cP, 4.6 cP, 4.5 cP, 4.4 cP, 4.3 cP, 4.2 cP, 4.1 cP, 4.0 cP, 3.9 cP, 3.8 cP, 3.7 cP, 3.6 cP, 3.5 cP, 3.4 cP, 3.3 cP, 3.2 cP, 3.1 cP, 3.0 cP, 2.9 cP, 2.8 cP, 2.7 cP, 2.6 cP, 2.5 cP, 2.4 cP, 2.3 cP, 2.2 cP, 2.1 cP, 2.0 cP, 1.9 cP, 1.8 cP, 1.7 cP, 1.6 cP, 1.5 cP, 1.4 cP, 1.3 cP, 1.2 cP, 1.1 cP, 1.0 cP, 0.9 cP, 0.8 cP, 0.7 cP, 0.6 cP or 0.5 cP. In some embodiments, the viscosity of an ASD forming solution, or a precursor solution thereof, is less than about 2 cP, such as less than about any of 1.95 cP, 1.9 cP, 1.85 cP, 1.8 cP, 1.75 cP, 1.7 cP, 1.65 cP, 1.6 cP, 1.55 cP, 1.5 cP, 1.45 cP, 1.4 cP, 1.35 cP, 1.3 cP, 1.25 cP, 1.2 cP, 1.15 cP, 1.1 cP, 1.05 cP, 1 cP, 0.95 cP, 0.9 cP, 0.85 cP, 0.8 cP, 0.75 cP, 0.7 cP, 0.65 cP, 0.6 cP, 0.55 cP, or 0.5 cP. In some embodiments, the viscosity of an ASD forming solution, or a precursor solution thereof, is at least about 0.5 cP, such as at least about any of 0.6 cP, 0.7 cP, 0.8 cP, 0.9 cP, 1.0 cP, 1.1 cP, 1.2 cP, 1.3 cP, 1.4 cP, 1.5 cP, 1.6 cP, 1.7 cP, 1.8 cP, 1.9 cP, 2.0 cP, 2.1 cP, 2.2 cP, 2.3 cP, 2.4 cP, 2.5 cP, 2.6 cP, 2.7 cP, 2.8 cP, 2.9 cP, 3.0 cP, 3.1 cP, 3.2 cP, 3.3 cP, 3.4 cP, 3.5 cP, 3.6 cP, 3.7 cP, 3.8 cP, 3.9 cP, 4.0 cP, 4.1 cP, 4.2 cP, 4.3 cP, 4.4 cP, 4.5 cP, 4.6 cP, 4.7 cP, 4.8 cP, 4.9 cP, or 5.0 cP. In some embodiments, the viscosity of an ASD forming solution, or a precursor solution thereof, is at least about 0.5 cP, such as at least about any of 0.55 cP, 0.6 cP, 0.65 cP, 0.7 cP, 0.75 cP, 0.8 cP, 0.85 cP, 0.9 cP, 0.95 cP, 1 cP, 1.05 cP, 1.1 cP, 1.15 cP, 1.2 cP, 1.25 cP, 1.3 cP, 1.35 cP, 1.4 cP, 1.45 cP, 1.5 cP, 1.55 cP, 1.6 cP, 1.65 cP, 1.7 cP, 1.75 cP, 1.8 cP, 1.85 cP, 1.9 cP, 1.95 cP, or 2.0 cP. In some embodiments, the viscosity of an ASD forming solution, or a precursor solution thereof, is about any of 0.5 cP, 0.6 cP, 0.7 cP, 0.8 cP, 0.9 cP, 1.0 cP, 1.1 cP, 1.2 cP, 1.3 cP, 1.4 cP, 1.5 cP, 1.6 cP, 1.7 cP, 1.8 cP, 1.9 cP, 2.0 cP, 2.1 cP, 2.2 cP, 2.3 cP, 2.4 cP, 2.5 cP, 2.6 cP, 2.7 cP, 2.8 cP, 2.9 cP, 3.0 cP, 3.1 cP, 3.2 cP, 3.3 cP, 3.4 cP, 3.5 cP, 3.6 cP, 3.7 cP, 3.8 cP, 3.9 cP, 4.0 cP, 4.1 cP, 4.2 cP, 4.3 cP, 4.4 cP, 4.5 cP, 4.6 cP, 4.7 cP, 4.8 cP, 4.9 cP, or 5.0 cP. In some embodiments, the viscosity of an ASD forming solution, or a precursor solution thereof, is about any of 0.5 cP, 0.55 cP, 0.6 cP, 0.65 cP, 0.7 cP, 0.75 cP, 0.8 cP, 0.85 cP, 0.9 cP, 0.95 cP, 1 cP, 1.05 cP, 1.1 cP, 1.15 cP, 1.2 cP, 1.25 cP, 1.3 cP, 1.35 cP, 1.4 cP, 1.45 cP, 1.5 cP, 1.55 cP, 1.6 cP, 1.65 cP, 1.7 cP, 1.75 cP, 1.8 cP, 1.85 cP, 1.9 cP, 1.95 cP, 2.0 cP, 2.05 cp, 2.1 cp, 2.15 cp, 2.2 cp, 2.25 cp, 2.3 cp, 2.35 cp, 2.4 cp, 2.45 cp, 2.5 cp, 2.55 cp, 2.6 cp, 2.65 cp, 2.7 cp, 2.75 cp, 2.8, 2.85 cp, 2.9 cp, 2.95 cp, or 3 cp. In the embodiments described herein, the viscosity of an ASD forming solution, or a precursor solution thereof, is assessed at about room temperature (such as about 20° C. to about 22° C., including at about any of 20° C., 21° C., or 22° C.). In some embodiments, wherein an ASD forming solution is produced from two or more precursor solutions thereof, at least two of the two or more precursor solutions have the same viscosity. In some embodiments, wherein an ASD forming solution is produced from two or more precursor solutions thereof, at least two of the two or more precursor solutions have a different viscosity.

Various components may, in some embodiments, contribute to the viscosity of an ASD forming solution, or a precursor solution thereof, including the type and/or concentration of a hydrophobic agent and/or polymer. In some embodiments, the ASD forming solution, or a precursor solution thereof, further comprises a viscosity-modulating agent. In some embodiments, the viscosity-modulating agent is a viscosity-enhancing agent, e.g., increases the viscosity of an ASD forming solution, or a precursor solution thereof. In some embodiments, the viscosity-modulating agent is a viscosity-reducing agent, e.g., reduces the viscosity of an ASD forming solution, or a precursor solution thereof.

In some embodiments, the viscosity-enhancing agent is selected from the group consisting of ethylene glycol, polyethylene glycol, glycerol, propylene glycerol, and a cellulosic polymer, or mixtures thereof. In some embodiments, the cellulosic polymer is selected from a methylcellulose, hydroxymethyl cellulose, and hydroxypropylmethyl cellulose, or mixtures thereof. In some embodiments, the viscosity-enhancing agent is a polymer useful for forming an ASD, such as discussed in other sections of the instant application.

In some embodiments, the viscosity-reducing agent is selected from the group consisting of a salt and an amino acid, or mixtures thereof.

In some embodiments, the viscosity-modulating agent, such as a viscosity-enhancing agent or a viscosity-reducing agent, has concentration in an ASD forming solution, or a precursor solution thereof, of about 0.1% v/v to about 40% v/v, such as any of about 1% v/v to about 10% v/v, about 5% v/v to about 15% v/v, about 10% v/v to about 20% v/v, about 15% v/v to about 25% v/v, about 20% v/v to about 30% v/v, about 25% v/v to about 35% v/v, or about 30% v/v to about 40% v/v. In some embodiments, the viscosity-modulating agent, such as a viscosity-enhancing agent or a viscosity-reducing agent, has concentration in an ASD forming solution, or a precursor solution thereof, of about 40% v/v or less, such as about any of 35% v/v, 30% v/v, 25% v/v, 20% v/v, 15% v/v, 10% v/v, 9% v/v, 8% v/v, 7% v/v, 6% v/v, 5% v/v, 4% v/v, 3% v/v, 2% v/v, 1% v/v, 0.9% v/v, 0.8% v/v, 0.7% v/v, 0.6% v/v, 0.5% v/v, 0.4% v/v, 0.3% v/v, 0.2% v/v, or 0.1% v/v. In some embodiments, the concentration of the viscosity-modulating agent, such as the viscosity-enhancing agent or a viscosity-reducing agent, in an ASD forming solution, or a precursor solution thereof, is based on a desired viscosity of the ASD forming solution, or a precursor solution thereof.

In some embodiments, the ratio of the viscosity-modulating agent, such as a viscosity-enhancing agent or a viscosity-reducing agent, to an organic carrier of an ASD forming solution, or a precursor solution thereof, is about 1:1000 to about 1:2, such as any of about 1:500 to about 1:2, about 1:100 to about 1:2, about 1:50 to about 1:2, about 1:20 to about 1:2, about 1:1000 to about 1:5, about 1:500 to about 1:5, about 1:100 to about 1:5, about 1:50 to about 1:5, about 1:20 to about 1:5, about 1:1000 to about 1:10, about 1:500 to about 1:10, about 1:100 to about 1:10, about 1:50 to about 1:10, or about 1:20 to about 1:10, or about 1:10 to about 1:5. In some embodiments, the ratio is about 1:5. In some embodiments, the ratio is about 1:10.

### E. Precursor Solutions

In certain aspects, provided herein is a precursor solution to an ASD forming solution. As described herein, methods of depositing an ASD forming solution onto a substrate comprises dispensing the ASD forming solution, or components thereof, from one or more droplet dispensers. In some embodiments, the ASD forming solution is formed via dispensing two or more precursor solutions comprising components thereof. For example, in some embodiments, the ASD forming solution may be formed via dispensing a first precursor solution comprising an agent susceptible to crystallization and dispensing a second precursor solution comprising a polymer, wherein the first precursor solution and the second precursor solution mix following dispensing to form the ASD forming solution. Accordingly, precursor solutions may contain components of an ASD forming solution in any configuration and/or concentration such that mixing of two or more precursor solutions results in an ADS forming solution.

In some embodiments, the precursor solution does not contain a component of an ASD forming solution. For example, in some embodiments, the ASD forming solution comprises an organic carrier, a polymer, and an agent susceptible to crystallization, wherein the ASD forming solution inhibits, such as prevents, the crystallization of the agent in the dried state. In some embodiments, the ADS forming solution optionally comprises a viscosity-modulating agent, such as a viscosity-modulating agent different from the polymer. In some embodiments, the precursor solution does not contain the organic carrier. In some embodiments, wherein the precursor solution does not contain the organic carrier, the precursor solution does not contain the agent susceptible to crystallization. In some embodiments, the precursor solution does not contain the agent susceptible to crystallization. In some embodiments, the precursor solution does not contain a viscosity-modulating agent.

In some embodiments, wherein the precursor solution does not contain an organic carrier, the precursor solution comprises any carrier suitable for use in a droplet dispenser. In some embodiments, the precursor solution comprises an aqueous carrier.

In some aspects provided herein, one or more precursor solutions are deposited onto a substrate and an ASD is not formed. For example, in some embodiments, a first precursor solution comprising an agent susceptible to crystallization and a second precursor solution comprising a polymer are deposited onto a substrate such that a gradient is formed comprising two or more different ratios of the agent to the polymer. In such embodiments, not all ratios, or not any, of the agent to the polymer will result in an ASD. In view of the disclosure provided herein, the term precursor solution is not intended to limit the scope of such solutions to those that form ASD forming solutions.

### IX. Methods of Identifying an ASD and/or ASD Forming Solution, and Products Thereof

In certain aspects, provided herein are methods of identifying an ASD and/or an ASD forming solution. ASDs are generally optimized to balance the amount of a polymer needed to inhibit crystallization of an agent and the amount of polymer needed to provide certain characteristic(s) associated with the agent, e.g., drug release from a drug dosage form comprising the ASD. Conventional identification of ASDs, and component ratios thereof, is a laborious process requiring large amounts of raw materials and high costs. In certain aspects, the methods provided herein comprise deposition of ASD forming solutions and/or precursor solutions, such as from one or more droplet dispensers, enabling both the identification of an ASD forming condition as well as an ASD having desired properties, such as a ratio of the agent to polymer having a desired release therefrom.

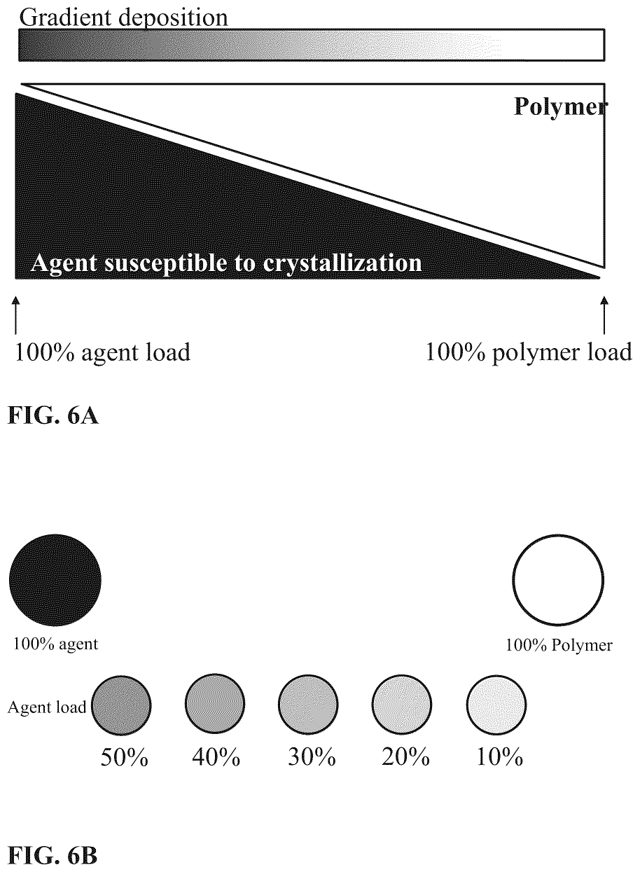

FIGS. 6A and 6B are provided for purposes of illustrating techniques for identifying an ASD and/or ASD forming solution. As shown in FIG. 6A, in some embodiments, a precursor solution comprising an agent susceptible to crystallization, such as a hydrophobic agent, and a precursor solution comprising a polymer suitable for ASD formation are dispensed from independent droplet dispensers. The amount of the first precursor solution dispensed from a first droplet dispenser and the amount of the second precursor solution dispensed from a second droplet dispenser can be varied across an area such that a gradient deposition is produced. The degree of variation can be controlled as desired. In FIG. 6A, the left side of the gradient deposition comprises 100% load of the agent susceptible to crystallization and the right side of the gradient deposition comprises 100% polymer load. The amount of each precursor solution deposited in the gradient deposition is illustrated with the black (agent) and white (polymer) triangles. The resulting gradient deposition can then be assessed to identify conditions suitable for forming an ASD comprising the agent. In some embodiments, provided herein is a product of the described methods, such as a gradient deposition comprising varying ratios of the agent susceptible to crystallization and the polymer. FIG. 6B illustrates another technique encompassed by the methods provided herein. Using a precursor solution comprising an agent susceptible to crystallization, such as a hydrophobic agent, and a precursor solution comprising a polymer suitable, depositions of an area (such as the illustrated circles) can be performed to produce specific agent loads. The resulting areas can then be assessed to identify conditions suitable for forming an ASD comprising the agent.

In some aspects, provided herein is a method of determining a ratio of an agent susceptible to crystallization, such as a hydrophobic agent, and a polymer that forms an ASD, the method comprising dispensing onto a substrate an amount of a first precursor solution comprising the agent, and an amount of a second precursor solution comprising the polymer to determine the ratio of the agent and the polymer that forms an ASD, wherein the dispensing is performed in such a manner that the first precursor solution and the second precursor solution are mixed on, or prior to being applied to, the substrate. In some embodiments, the method comprises generating a plurality of areas on the substrate each having a different ratio of the agent to the polymer from the precursor solutions. In some embodiments, the different areas form a gradient of ratios of the agent and polymer, such as from 0% agent and 100% polymer to 100% agent and 0% polymer. In some embodiments, the first precursor solution comprises an organic carrier and the agent. In some embodiments, the first precursor solution further comprises the polymer. In some embodiments, the first precursor solution further comprises a viscosity-enhancing agent. In some embodiments, the second precursor solution comprises the polymer and another carrier, such as the organic carrier. In some embodiments, the second precursor solution comprises another viscosity-enhancing agent.

In some embodiments, the first precursor solution is dispensed from a first droplet dispenser, such as a sub-nanoliter droplet dispenser, e.g., a high-frequency, sub-nanoliter dispenser. In some embodiments, the second precursor solution is dispensed from a second droplet dispenser, such as a sub-nanoliter droplet dispenser, e.g., a high-frequency, sub-nanoliter dispenser.

In some embodiments, the method comprises admixing components of a first precursor solution and a second precursor solution, such as to obtain an ASD forming solution, or a candidate thereof, and dispensing the ASD forming solution, or the candidate thereof, onto a substrate using a droplet dispenser to determine if an ASD is formed. In some embodiments, a plurality of ADS forming solutions are independently deposited on a substrate to determine a ratio of an agent susceptible to crystallization, such as a hydrophobic agent, and a polymer that forms an ASD.

In some embodiments, the method further comprises assessing the crystallization of the agent susceptible to crystallization, such as a hydrophobic agent.

In some embodiments, the substrate is a thin film, such as a pharmaceutical thin film substrate described herein.

In certain aspects, provided is a gradient on a substrate, the gradient comprising: a first position, a second position, and a third position each comprising a mixture of an amount of a first deposit and an amount of a second deposit, wherein the first deposit comprises an agent susceptible to crystallization, such as a hydrophobic agent, wherein the ratio of the amount of the first deposit to the amount of the second deposit in said first position is less than the ratio of the amount of the first deposit to the amount of the second deposit in said second position, and the ratio of the amount of the first deposit to the amount of the second deposit in said second position is less than the ratio of amount of the first deposit to the amount of the second deposit in said third position, wherein the interaction between the first deposit and the second deposit in the mixture at the first position, the second position, and the third position produces a measureable effect of the agent of the first deposit. In some embodiments, the difference in the ratios of the amount of the first deposit to the amount of the second deposit in each of the first position, second position and third position is the same.

In some embodiments, the first deposit comprises a hydrophobic deposit and the second deposit comprises a polymer. In some embodiments, the first deposit further comprises a first organic carrier and a first viscosity-enhancing agent, and the second deposit further comprises a second organic carrier and a second viscosity-enhancing agent. In some embodiments, the first organic carrier and the second organic carrier are the same. In some embodiments, the first viscosity-enhancing agent and the second viscosity-enhancing agent are the same. In some embodiments, the amount of the first deposit and the amount of the second deposit is a concentration. In some embodiments, the measureable effect of the hydrophobic agent is crystallinity. In some embodiments, the first deposit is dispensed from a first droplet dispenser, such as a sub-nanoliter droplet dispenser, and wherein the second deposit is dispensed from a second droplet dispenser, such as a sub-nanoliter droplet dispenser.

### X. Methods of Making a Dosage Form Comprising an ASD

In some aspects, provided herein is a method of making a dosage form comprising an amorphous solid dispersion (ASD) comprising an agent susceptible to crystallization, e.g., a hydrophobic agent, the method comprising depositing droplets of an ASD forming solution comprising the agent, an organic carrier, and a polymer onto a substrate using a droplet dispenser, wherein the ratio of the polymer and the agent inhibits crystallization of the agent in a dried state on the substrate. In some embodiments, depositing droplets of an ASD forming solution onto a substrate comprises dispensing the ASD forming solution from a droplet dispenser. In some embodiments, depositing droplets of an ASD forming solution onto a substrate is performed via dispensing the ASD forming solution and/or precursor solutions thereof from more than one droplet dispenser, wherein the ASD forming solution is formed on the substrate and/or during travel from the droplet dispenser(s) to the substrate. In some embodiments, the ASD forming solution precursor comprises a subset of components of an ASD forming solution. In some embodiments, the ASD forming solution precursor comprises the components of an ASD forming solution at a different concentration than in a resulting ASD forming solution. In some embodiments, different ASD forming solutions and/or precursor solutions thereof may each be dispensed from a different droplet dispenser.

In the methods provided herein, the droplet dispenser ejects a droplet which travels to a substrate. Many configurations of methods are provided herein based on, e.g., the configuration of one or more droplet dispensers and an ASD forming solution. In some embodiments, the ASD forming solution is formed during travel from a droplet dispenser to a substrate. In some embodiments, the ASD forming solution is formed once deposited on a substrate by a droplet dispenser. In some embodiments, the ASD forming solution is formed during travel from a droplet dispenser to a substrate and once deposited on a substrate by a droplet dispenser. In some embodiments, the ASD is formed during travel from a droplet dispenser to a substrate. In some embodiments, the ASD is formed once deposited on a substrate by a droplet dispenser. In some embodiments, the ASD is formed during travel from a droplet dispenser to a substrate and once deposited on a substrate by a droplet dispenser.

In some embodiments, the method comprises depositing an ASD forming solution onto a substrate, wherein the ASD forming solution was identified using the methods described herein. The methods provided herein may utilize any component described herein, such as an ASD forming solution and/or precursor solutions thereof, droplet dispensers, and substrates. The modular fashion of description is not intended to limit the scope of the teachings provided herein, and one of ordinary skill in the art will readily appreciate that the methods encompass the embodiments described therein and any combination thereof.

In some embodiments, the method comprises depositing droplets of an ASD forming solution comprising an agent susceptible to crystallization, such as a hydrophobic agent, an organic carrier, and a polymer onto a substrate using a droplet dispenser, wherein the ratio of the polymer and the agent susceptible to crystallization inhibits crystallization of the agent in a dried state on the substrate. In some embodiments, depositing droplets of the ASD forming solution comprises dispensing the ASD forming solution from a droplet dispenser. In some embodiments, depositing droplets of the ASD forming solution comprises dispensing a precursor solution from a droplet dispenser. In some embodiments, the ASD forming solution, or a precursor solution thereof, has a viscosity of about 0.5 cp to about 5 cP, including about 1 cp to less than 2 cp or about 1.2 cP to about 1.8 cP. In some embodiments, the droplet dispenser is a sub-nanoliter droplet dispenser. In some embodiments, the droplet dispenser is a high-frequency, sub-nanoliter droplet dispenser. In some embodiments, the ASD forming solution, or a precursor solution thereof, is sourced from a dispensing cartridge. In some embodiments, the method comprises conveying an ASD forming solution, or a precursor solution thereof, to a droplet dispenser or a component thereof, such as a dispensing head thereof.

In some embodiments, the droplet dispenser dispenses droplets in the direction of a substrate to deposit the droplets thereon. Generally speaking, the dispensed droplets from the droplet dispenser may experience changes in form during the deposition process, e.g., (a) one droplet may merge with another droplet after being dispensed from the droplet dispenser (whether in the air or on the substrate), (b) deposited droplets dry on the substrate, and (c) loss of solvent during travel from the droplet dispenser to the substrate. Description of droplets is provided herein and may be applicable to the various states that occur during the deposition process. Such description is not intended to be limited to only a certain state unless otherwise expressly noted. In some embodiments, the depositing comprises dispensing a number of droplets of an ASD forming solution per square inch (also referred to herein as dots per inch). In some embodiments, the resulting pattern or patterned layer produced by a method described herein comprises, at least to a degree, merging of one or more dispensed drops of an ASD forming solution. Such a pattern of droplets need not be regular, and in some embodiments, reflects a number of droplets applied to a set area of a substrate. In some embodiments, the deposited droplets (whether or not merged prior to or upon the substrate) resemble a pattern on a macroscale, such as a printed shape. In some embodiments, the dosage form comprises one or more layers of droplets deposited on a substrate, such as formed from one or more passes of a droplet dispenser over an area of a dosage form. In some embodiments, the patterned layer, prior to drying, is a continuous layer of the ADS forming solution, e.g., the deposited droplets are interconnected to at least a degree. In some embodiments, the droplets of an ADS forming solution are deposited at about 1 dot per inch (dpi) to about 1,000 dpi, such as any of about 1 dpi to about 720 dpi, about 72 dpi to about 720 dpi, about 72 dpi to about 500 dpi, or about 72 dpi to about 300 dpi. In some embodiments, the droplets of an ASD forming solution are deposited at about 1 dpi or greater, such as about any of 20 dpi or greater, 50 dpi or greater, 72 dpi or greater, 100 dpi or greater, 120 dpi or greater, 150 dpi or greater, 200 dpi or greater, 250 dpi or greater, 300 dpi or greater, 350 dpi or greater, 400 dpi or greater, 450 dpi or greater, 500 dpi or greater, 550 dpi or greater, 600 dpi or greater, 650 dpi or greater, 700 dpi or greater, 720 dpi or greater, 750 dpi or greater, 800 dpi or greater, 850 dpi or greater, 900 dpi or greater, 950 dpi or greater, or 1,000 dpi or greater. In some embodiments, the droplets of an ASD forming solution are deposited at about 1 dot per inch (dpi) to about 1,000 dpi, such as any of about 1 dpi to about 720 dpi, about 72 dpi to about 720 dpi, about 72 dpi to about 500 dpi, or about 72 dpi to about 300 dpi. In some embodiments, the droplets of an ASD forming solution are deposited at about 1 dpi or greater, such as about any of 20 dpi or greater, 50 dpi or greater, 72 dpi or greater, 100 dpi or greater, 120 dpi or greater, 150 dpi or greater, 200 dpi or greater, 250 dpi or greater, 300 dpi or greater, 350 dpi or greater, 400 dpi or greater, 450 dpi or greater, 500 dpi or greater, 550 dpi or greater, 600 dpi or greater, 650 dpi or greater, 700 dpi or greater, 720 dpi or greater, 750 dpi or greater, 800 dpi or greater, 850 dpi or greater, 900 dpi or greater, 950 dpi or greater, or 1,000 dpi or greater.

In some embodiments, the dispensed or deposited droplet of an ASD forming solution, or a precursor solution thereof, has a diameter of about 1 nm to about 200 μm, such as any of about 20 nm to about 800 nm, about 100 nm to about 500 nm, or about 10 μm to about 100 μm. In some embodiments, the dispensed or deposited droplet of an ASD forming solution, or a precursor solution thereof, has a diameter of at least about 1 nm, such as at least about any of 10 nm, 20 nm, 30 nm, 40 nm, 50 nm, 60 nm, 70 nm, 80 nm, 90 nm, 100 nm, 110 nm, 120 nm, 130 nm, 140 nm, 150 nm, 160 nm, 170 nm, 180 nm, 190 nm, 200 nm, 210 nm, 220 nm, 230 nm, 240 nm, 250 nm, 260 nm, 270 nm, 280 nm, 290 nm, 300 nm, 310 nm, 320 nm, 330 nm, 340 nm, 350 nm, 360 nm, 370 nm, 380 nm, 390 nm, 400 nm, 410 nm, 420 nm, 430 nm, 440 nm, 450 nm, 460 nm, 470 nm, 480 nm, 490 nm, 500 nm, 525 nm, 550 nm, 575 nm, 600 nm, 625 nm, 650 nm, 675 nm, 700 nm, 725 nm, 750 nm, 775 nm, 800 nm, 825 nm, 850 nm, 875 nm, 900 nm, 925 nm, 950 nm, 975 nm, 1 μm, 5 μm, 10 μm, 15 μm, 20 μm, 25 μm, 30 μm, 35 μm, 40 μm, 45 μm, 50 μm, 60 μm, 70 μm, 80 μm, 90 μm, or 100 μm. In some embodiments, the average of the population of dispensed or deposited droplets of a dosage form described herein meet the criteria discussed above.

In some embodiments, the dispensed or deposited droplets of an ASD forming solution, or a precursor solution thereof, comprise an average volume of about 1 picoliter (pL) to about 1 nL, such as about 1 pL to about 500 pL or about 1 pL to about 50 pL. In some embodiments, the dispensed or deposited droplets of an ASD forming solution, or a precursor solution thereof, comprises comprise an average volume of about 1 nL or less, such as about any of 950 pL or less, 900 pL or less, 850 pL or less, 800 pL or less, 750 pL or less, 700 pL or less, 650 pL or less, 600 pL or less, 550 pL or less, 500 pL or less, 450 pL or less, 400 pL or less, 350 pL or less, 300 pL or less, 250 pL or less, 200 pL or less, 150 pL or less, 100 pL or less, 75 pL or less, 50 pL or less, or 25 pL or less.

As described in more detail in other sections herein, in some embodiments, the droplet dispenser dispenses an ASD forming solution, or a precursor solution thereof, using a thermal process. In some embodiments, the droplet dispenser dispenses an ASD forming solution, or a precursor solution thereof, using a piezoelectric process. In some embodiments, the droplet dispenser is a sub-nanoliter droplet dispenser. In some embodiments, such as described in more detail in other section herein, the sub-nanoliter droplet dispenser is a high frequency, sub-nanoliter droplet dispenser. In some embodiments, the droplet dispenser comprises about 100 to about 1000 individual nozzles. In some embodiments, each nozzle comprises an aperture with a diameter of about 20 μm to about 120 μm. In some embodiments, the high frequency, sub-nanoliter droplet dispenser dispenses droplets at more than 3 kHz as measured per nozzle.

In some embodiments, the method further comprises coupling a cartridge containing an ASD forming solution, or a precursor solution thereof, and a droplet dispenser (such as a high frequency droplet dispenser or high frequency sub-nanoliter droplet dispenser). In some embodiments, the method further comprises selecting a cartridge comprises an ASD forming solution, or a precursor solution thereof, to produce a desired dosage form, such as based on one or more of a desired characteristics of the final dosage form or one or more patterned layers thereon, e.g., amount of a hydrophobic agent to be deposited on the dosage form, amount of a polymer to be deposited on the dosage form, a characteristic of a patterned layer (such as any of dpi, shape, size, and position on the dosage form), a release profile of a hydrophobic agent, and a feature of the final dosage form (e.g., structural integrity and/or protection of the patterned layer). In certain embodiments, the method comprises dispensing or depositing at least one ASD forming solution and, optionally, another material (e.g., a food safe material to form a label), wherein the methods encompass selection of the each dispensed component. In some embodiments, the method comprises selecting an ASD forming solution, or a precursor solution thereof, from a library (such as compiled in a kit). In some embodiments, the method further comprises making an ASD forming solution and/or a precursor solution thereof.

In some embodiments, the method comprises designing a pattern, such as characterized by dpi, of droplets of an ASD forming solution comprising an agent susceptible to crystallization, e.g., a hydrophobic agent. In some embodiments, the method of designing comprises determining (such as calculating) one or more settings based on any one or more of: the total amount of the agent of the dosage form, such as a hydrophobic agent; the concentration of the agent in the ASD forming solution, or a precursor solution thereof; the desired surface area of exposure of the resulting ASD, or a dried form thereof, of the dosage form; the desired release profile of the agent; the amount of polymer relative to the agent; and a characteristic of the droplet dispenser (or use thereof), such as aperture diameter, ejection frequency, and produced drop size.

In some embodiments, the method comprises creating and/or receiving instructions to control the droplet dispenser, such as to form a dosage form described herein. In some embodiments, the instructions are personalized for an individual.

In some embodiments, the method further comprises performing a drying time following depositing of an ASD forming solution on a substrate, e.g., such that the ASD forming solution can dry, at least to a degree, and/or the ASD can be formed. In some embodiments, the method further comprises one or more processing steps following depositing of an ASD forming solution on a substrate, such as cutting a substrate into individual dosage forms and/or packing one or more dosage forms.

In some embodiments, the method further comprises cleaning one or more dispensing head of a droplet dispenser, such as by dispensing a non-drug containing solvent. For example, such cleaning steps help to ensure that an ASD forming solution, or a precursor solution thereof, has not clogged a nozzle of a dispensing head and/or to reduce carry-over of a component dispensed therefrom.

### XI. ASD Dispensing Cartridges and Compositions Therein

Provided herein, in certain aspects, is a dispensing cartridge comprising (or suitable for use with) an ASD forming solution and/or a precursor solution thereof described herein, or a derivative or precursor thereof. The dispensing cartridges described herein are generally suitable to hold one or more ASD forming solutions and/or a precursor solutions thereof described herein, such as in a reservoir of a dispensing cartridge, and are configured to interface with a dispensing head of a droplet dispenser such that the contents of the dispensing cartridge can be delivered to the dispensing head. In some embodiments, the dispensing cartridge is a disposable cartridge. In some embodiments, the dispensing cartridge is suitable for one-time or limited-time use.

In some embodiments, the dispensing cartridge comprises a material suitable for use with an ASD forming solution and/or a precursor solution thereof described herein. In some embodiments, the dispensing cartridge comprises a material suitable for use in a pharmaceutical application, e.g., the material does not dissolve or leach a component thereof in the presence of an ASD forming solution and/or a precursor solution thereof.

In some embodiments, the dispensing cartridge comprises one reservoir configured to hold an ASD forming solution and/or a precursor solution thereof described herein. In some embodiments, the dispensing cartridge comprises more than one reservoir configured to hold ASD forming solution(s) and/or a precursor solution(s) thereof described herein. In some embodiments, provided is a dispensing cartridge comprising a material useful for an embodiment described herein, wherein the material is not an ASD forming solution and/or a precursor solution thereof described herein. For example, in some embodiments, the dispensing cartridge comprises a fluid suitable for depositing onto a substrate, such as to communicate a feature or identity (including unique identity) of a dosage form via a logo and/or label. In some embodiments, the label is a machine-readable label, such as a QR code or a barcode. Also disclosed herein are kits comprising one or more dispensing cartridges described herein.

In some embodiments, the dispensing cartridge comprises a reservoir, such as to contain a volume of an ASD forming solution and/or a precursor solution thereof described herein. In some embodiments, the dispensing cartridge comprises a reservoir port, such as facilitate the flow of material(s) into or out of a reservoir. In some embodiments, the dispensing cartridge comprises a sponge material. In some embodiments, the sponge material is in a reservoir. In some embodiments, the dispensing cartridge comprises a dispensing head interface configured to enable the passage of an ASD forming solution and/or a precursor solution thereof from a reservoir to a dispensing head of a droplet dispenser.

In some embodiments, provided herein is a composition comprising a form of an ASD forming solution and/or a precursor solution thereof, including a derivative or precursor thereof. In some embodiments, the form of an ASD forming solution and/or a precursor solution thereof is a frozen form, a dried form, or a lyophilized form. In some embodiments, the form of an ASD forming solution and/or a precursor solution thereof can be processed to produce the desired ASD forming solution and/or a precursor solution thereof described herein, such as via reconstitution. In some embodiments, the composition is contained in a dispensing cartridge.

### XII. ASD Dosage Forms

Provided herein, in certain aspects, is a dosage form comprising an ASD comprising an agent susceptible to crystallization, such as a hydrophobic agent, deposited onto a substrate. In some embodiments, provided herein is a dosage form produced according to the methods provided herein. In some embodiments, the dosage form has different mechanical properties as compared to a dosage from produced via conventional means, such as solvent casting.

In some embodiments, the agent susceptible to crystallization is present in the dosage form with less than 1%, 2%, 3%, 4%, 5%, 6%, 7%, 8%, 9%, or 10% of the agent in crystalline form. In some embodiments, the agent susceptible to crystallization is present in the dosage form with less than 5% of the agent in crystalline form. In some embodiments, substantially no crystallized form of the agent susceptible to crystallization is present in the dosage form.

In some embodiments, the dosage form is an oral dosage form (including an oral drug dosage form such as a capsule comprising a thin film substrate, e.g., a rolled and/or folded thin film substrate). In some embodiments, the dosage form is an intra-oral dosage form. In some embodiments, the dosage form is a buccal dosage form. In some embodiments, the dosage form is a sublingual dosage form. In some embodiments, the dosage form is a vaginal dosage form. In some embodiments, the dosage form is a rectal dosage form. In some embodiments, the dosage form is an ocular dosage from. In some embodiments, the dosage form comprises a component derived from deposition of a described ASD forming solution using the methods provided herein. In some embodiments, the thin film substrate is suitable for human consumption. In some embodiments, the dosage form is suitable for contact-based delivery of a hydrophobic agent, such a transdermal dosage form. In some embodiments, the dosage form is an ocular dosage form. In some embodiments, the thin film substrate comprises a polymer (e.g., the thin film substrate is a polymer film). In some embodiments, the thin film substrate comprises one or more of chitosan, ethylenediaminetetraacetic acid, polyvinyl pyrrolidone, polyvinyl alcohol, alginate, agar, carrageenan, guar gum, xanthan gum, polycarbophil, and polyacrylic acid derivatives.

The dosage forms described herein may have a patterned layer of any shape and/or size. In some embodiments, characteristics of a patterned layer are based on an amount of an agent susceptible to crystallization, such as a hydrophobic agent, of a dosage form. For example, such characteristics of a patterned layer may be based on the hydrophobic agent concentration of an ASD forming solution and the total desired amount of the hydrophobic agent to be deposited onto a substrate, such as a thin film substrate. In some embodiments, the characteristic of a patterned layer (such as any of shape, size, and position on the dosage form) are based on a desired release profile of an agent susceptible to crystallization, such as a hydrophobic agent (e.g., a patterned layer may be designed to increase surface area to a bodily fluid) and/or feature of the final dosage form (e.g., structural integrity and/or protection of the patterned layer). In some embodiments, the term layer is not to be construed as to mean only a single deposition from a droplet dispenser described herein. In some embodiments, a patterned layer is formed from many instances of droplet deposition from a droplet dispenser described herein (e.g., repeated printing of an area using an ASD forming solution and/or a precursor solution thereof described herein). For example, in producing a dosage form comprising a patterned layer, a droplet dispenser may be controlled to make one or more passes to deposit droplets of an ASD forming solution and/or a precursor solution described herein to form the final patterned layer.

In some embodiments, the dosage form comprises an ASD forming solution deposited onto a substrate at about 1 dot per inch (dpi) to about 1,000 dpi, such as any of about 1 dpi to about 720 dpi, about 72 dpi to about 720 dpi, about 72 dpi to about 500 dpi, or about 72 dpi to about 300 dpi. In some embodiments, the dosage form comprises an ASD forming solution deposited onto a substrate at about 1 dpi or greater, such as about any of 20 dpi or greater, 50 dpi or greater, 72 dpi or greater, 100 dpi or greater, 120 dpi or greater, 150 dpi or greater, 200 dpi or greater, 250 dpi or greater, 300 dpi or greater, 350 dpi or greater, 400 dpi or greater, 450 dpi or greater, 500 dpi or greater, 550 dpi or greater, 600 dpi or greater, 650 dpi or greater, 700 dpi or greater, 720 dpi or greater, 750 dpi or greater, 800 dpi or greater, 850 dpi or greater, 900 dpi or greater, 950 dpi or greater, or 1,000 dpi or greater. In some embodiments, the dosage form comprises an ASD forming solution deposited onto a substrate at about 1 dot per inch (dpi) to about 1,000 dpi, such as any of about 1 dpi to about 720 dpi, about 72 dpi to about 720 dpi, about 72 dpi to about 500 dpi, or about 72 dpi to about 300 dpi. In some embodiments, the dosage form comprises an ASD forming solution deposited onto a substrate at about 1 dpi or greater, such as about any of 20 dpi or greater, 50 dpi or greater, 72 dpi or greater, 100 dpi or greater, 120 dpi or greater, 150 dpi or greater, 200 dpi or greater, 250 dpi or greater, 300 dpi or greater, 350 dpi or greater, 400 dpi or greater, 450 dpi or greater, 500 dpi or greater, 550 dpi or greater, 600 dpi or greater, 650 dpi or greater, 700 dpi or greater, 720 dpi or greater, 750 dpi or greater, 800 dpi or greater, 850 dpi or greater, 900 dpi or greater, 950 dpi or greater, or 1,000 dpi or greater.

In some embodiments, the deposited droplet of an ASD forming solution has a diameter (or largest crossing dimension) of about 1 nm to about 200 μm, such as any of about 20 nm to about 800 nm, about 100 nm to about 500 nm, or about 10 μm to about 100 μm. In some embodiments, the deposited droplet of an ASD forming solution has a diameter (or largest crossing dimension) of at least about 1 nm, such as at least about any of 10 nm, 20 nm, 30 nm, 40 nm, 50 nm, 60 nm, 70 nm, 80 nm, 90 nm, 100 nm, 110 nm, 120 nm, 130 nm, 140 nm, 150 nm, 160 nm, 170 nm, 180 nm, 190 nm, 200 nm, 210 nm, 220 nm, 230 nm, 240 nm, 250 nm, 260 nm, 270 nm, 280 nm, 290 nm, 300 nm, 310 nm, 320 nm, 330 nm, 340 nm, 350 nm, 360 nm, 370 nm, 380 nm, 390 nm, 400 nm, 410 nm, 420 nm, 430 nm, 440 nm, 450 nm, 460 nm, 470 nm, 480 nm, 490 nm, 500 nm, 525 nm, 550 nm, 575 nm, 600 nm, 625 nm, 650 nm, 675 nm, 700 nm, 725 nm, 750 nm, 775 nm, 800 nm, 825 nm, 850 nm, 875 nm, 900 nm, 925 nm, 950 nm, 975 nm, 1 μm, 5 μm, 10 μm, 15 μm, 20 μm, 25 μm, 30 μm, 35 μm, 40 μm, 45 μm, 50 μm, 60 μm, 70 μm, 80 μm, 90 μm, or 100 μm. In some embodiments, the average of the population of droplets (such as a patterned layer(s)) described herein meet the criteria discussed above.

In some embodiments, the patterned layer is, at least to a degree, within the substrate, such as a thin film substrate (e.g., at least a portion of an ASD described herein penetrates and/or is absorbed into the thin film substrate). In some embodiments, the patterned layer is, at least to a degree, on top of the substrate, such as a thin film substrate (e.g., at least a portion of an ASD described herein is at or above the surface of the thin film substrate). In some embodiments, the patterned layer is, at least to a degree, within the substrate, such as a thin film substrate, and is, at least to a degree, on top of the substrate.

In some embodiments, the dosage form comprises more than one patterned layer, e.g., a first patterned layer and a second patterned layer. In some embodiments, the first patterned layer comprises an ASD comprising an agent susceptible to crystallization, such as a hydrophobic agent, and the second patterned layer comprises another agent. In some embodiments, the first patterned layer comprises an ASD comprising an agent susceptible to crystallization, such as a hydrophobic agent, and the second patterned layer comprises an ASD comprising the agent. In some embodiments, the first patterned layer comprises an ASD comprising an agent susceptible to crystallization, such as a hydrophobic agent, and the second patterned layer comprises an ASD comprising a second agent susceptible to crystallization, such as a second hydrophobic agent. In some embodiments, each patterned layer is produced via a layer-by-layer deposition technique.

In some embodiments, the dosage form comprises an amount of the agent susceptible to crystallization, such as a hydrophobic agent, of about 1 ng to about 1 mg, such as about 100 ng to about 500 ng, about 250 ng to about 750 ng, about 500 ng to about 1 pg, about 750 ng to about 1.25 pg, about 1 μg to about 2 pg, about 1 μg to about 10 pg, about 10 μg to about 50 pg, or about 25 μg to about 100 pg.

In some embodiments, the dosage form comprises an amount of the agent susceptible to crystallization, such as a hydrophobic agent, of at least about 1 ng, such as at least about any of 5 ng, 10 ng, 20 ng, 30 ng, 40 ng, 50 ng, 60 ng, 70 ng, 80 ng, 90 ng, 100 ng, 125 ng, 150 ng, 175 ng, 200 ng, 225 ng, 250 ng, 275 ng, 300 ng, 325 ng, 350 ng, 375 ng, 400 ng, 425 ng, 450 ng, 475 ng, 500 ng, 525 ng, 550 ng, 575 ng, 600 ng, 625 ng, 650 ng, 675 ng, 700 ng, 725 ng, 750 ng, 775 ng, 800 ng, 825 ng, 850 ng, 875 ng, 900 ng, 925 ng, 950 ng, 975 ng, 1 μg, 2 pg, 3 μg, 4 pg, 5 pg, 6 μg, 7 pg, 8 μg, 9 pg, 10 μg, 15 pg, 20 μg, 25 pg, 30 μg, 35 pg, 40 μg, 45 pg, 50 μg, 55 pg, 60 μg, 65 pg, 70 μg, 75 pg, 80 μg, 85 pg, 90 μg, 95 pg, or 100 pg.

In some embodiments, the dosage form further comprises a marking and/or a label. For example, in certain embodiments, the dosage form may contain printed information pertaining to any one or more of the active agent, amount of the active agent(s), the date produced, the individual the dosage form is produced for, the point of production, a trackable identification code, an expiration date, instructions for use, instructions for storage, or a company name and/or logo. In some embodiments, the marking and/or label is suitable for consumption, e.g., comprises a food safe dye.

In some embodiments, the dosage form comprises a protective coating, e.g., applied over a deposited ASD or a portion thereof. In some embodiments, the protective coating is applied to a dosage from via a droplet dispenser, such as a sub-nanoliter droplet dispenser or a high frequency, sub-nanoliter droplet dispenser. In some embodiments, the dosage form comprises a film coating.

In some embodiments, the dosage form is suitable for human use and/or consumption. In some embodiments, the dosage form is sterile.

In certain aspects, provided here is a batch of dosage forms described herein. In some embodiments, the batch of dosage forms comprises at least 5 dosage forms, such as any of 10, 20, 30, 40, 50, 60, 70, 80, 90, or 100 dosage forms. In some embodiments, the batch of a drug dosage form described comprises individual dosage forms having an amount of an agent susceptible to crystallization, such as a hydrophobic agent, that does not vary more than 5% from the average.

### XIII. Devices for Dispensing and Depositing ASDs

Provided herein, in certain aspects, are devices for dispensing and depositing droplets, such as droplets of an ASD forming solution and/or a precursor solution thereof described herein. In some embodiments, the device comprises a sub-nanoliter droplet dispenser. In some embodiments, the device comprises a high frequency, sub-nanoliter droplet dispenser. Sub-nanoliter droplet dispensers, including sub-nanoliter, high frequency droplet dispensers, are known in the art, including as described in U.S. Pat. Nos. 7,585,038, 7,963,635, and 6,851,786, which are hereby incorporated by reference in their entirety.

In some embodiments, provided is a droplet dispenser, such as a sub-nanoliter droplet dispenser or high frequency, sub-nanoliter droplet dispenser, for producing a dosage form, the dispenser comprising: (a) a dispensing head operably connected to a reservoir for an ASD forming solution or a precursor solution thereof; and (b) a control system configured to control the deposition of the ASD forming solution or the precursor solution thereof via the dispensing head. In some embodiments, provided is a droplet dispenser, such as a sub-nanoliter droplet dispenser or high frequency, sub-nanoliter droplet dispenser, for producing a dosage form, the dispenser comprising: (a) a dispensing head; (b) an ASD forming solution or a precursor solution thereof dispensing cartridge receiving space, wherein the dispensing cartridge receiving space is configured to operably couple a dispensing cartridge to the dispensing head; and (c) a control system configured to control the deposition of an ASD forming solution or a precursor solution thereof via the dispensing head according to a dispensing configuration, such as a dpi characteristic. In some embodiments, the viscosity of the ASD forming solution or the precursor solution thereof is about 0.5 cp to about 5 cp, such as about 1 cp to about 2 cp or about 1.8 cP and about 1.2 cP.

In some embodiments, the dispensing head comprises about 10 to about 1000 individual nozzles. In some embodiments, the dispensing head comprises at least about 10, such as at least about any of 20, 30, 40, 50, 60, 70, 80, 90, 100, 150, 200, 250, 300, 350, 400, 450, 500, 550, 600, 650, 700, 750, 800, 850, 900, 950 or 1,000, individual nozzles.

In some embodiments, each nozzle of a dispensing head has an aperture (where the nano-suspension is dispensed from) with a largest cross-section (such as diameter) of about 20 μm to about 120 μm, such as about 20 μm to about 70 μm or about 30 μm to about 60 μm. In some embodiments, the nozzle of a dispensing head has an aperture with a largest cross-section (such as diameter) of at least about 20 μm, such as at least about any of 30 μm, 35 μm, 40 μm, 45 μm, 50 μm, 55 μm, 60 μm, 75 μm, 100 μm, or 120 μm.

In some embodiments, each nozzle of a dispensing head is configured to dispense a droplet of an ASD forming solution or a precursor solution thereof having a volume of about 1 to about 500 picoliters (pL). In some embodiments, the nozzle of a dispensing head is configured to dispense a droplet of an ASD forming solution or a precursor solution thereof having a volume of about 500 pL or less, such as about any of 450 pL or less, 400 pL or less, 350 pL or less, 300 pL or less, 250 pL or less, 200 pL or less, 150 pL or less, 100 pL or less, 75 pL or less, 50 pL or less, or 25 pL or less.

In some embodiments, the dispensing head is a continuous dispensing head. In some embodiments, the dispensing head is a drop-on-demand printing head. In some embodiments, the droplet dispenser dispenses the droplets using a thermal process. In some embodiments, the droplet dispenser dispenses the droplets using a piezoelectric process. In some embodiments, the dispensing head is a thermal inkjet print head. In some embodiments, the dispensing head is a piezoelectric inkjet print head. In some embodiments, the dispensing head is a solenoid valve printing head. In some embodiments, the dispensing head is an electrostatic printing head. In some embodiments, the dispensing head is an acoustic printing head.

In some embodiments, the dispensing head is a sub-nanoliter dispensing head. In some embodiments, the dispensing head is a high frequency sub-nanoliter dispensing head (such as a dispensing head operating at an ejection frequency of above 2,000 Hz, such as above about 2.5 kHz, 3 kHz, 3.5 kHz, 4 kHz, 4.5 kHz, or 5 kHz).

In some embodiments, the dispensing device comprises more than one dispensing head. In some embodiments, the dispensing device further comprises a second dispensing head operably coupled to a second dispensing cartridge. In some embodiments, the dispensing device further comprises a third dispensing head operably coupled to a third dispensing cartridge. In some embodiments, the dispensing device further comprises a fourth dispensing head operably coupled to a fourth dispensing cartridge. In configurations comprising more than one dispensing head, the dispensing device is configured to be operably coupled to dispensing cartridges comprising any variety of materials, such as nano-suspensions, protective coatings, and label and/or marking materials (such as an edible ink).

### XIV. Systems and Applications Comprising ASD Embodiments Described Herein

Provided herein, in certain aspects, are systems and applications utilizing a method of making a dosage form described herein, and features associated therewith. In some embodiments, provided is a system comprises a dispensing device configured to receive instructions for printing a dosage form described herein. In some embodiments, the system is established at a point-of-care, such as in a hospital, care facility, pharmacy, or home. In some embodiments, the dosage form is a personalized dosage form, e.g., designed for an individual based on dosage form features such as the amount of a hydrophobic agent.

In some embodiments, the methods and dispensing devices described herein are suitable for personalized medicine. In some embodiments, the methods and dispensing devices described herein are suitable for point-of-care application. In some embodiments, the methods and dispensing devices described herein are suitable for rapid dosage form production (including on a small scale, such as less than 100 dosage forms). In some embodiments, the methods and dispensing devices described herein are suitable for producing dosage forms having a precise and accurate amount of an agent susceptible to crystallization, such as a hydrophobic agent (such as required for agents having a narrow therapeutic index, high potency, and/or pediatric use), in the form of an ASD. In some embodiments, the methods and dispensing devices described herein are suitable for producing dosage forms having an agent susceptible to crystallization, such as a hydrophobic agent, having poor stability (such as not stable for extended periods of time following production, e.g., greater than 24 hours) in the form of an ASD. In some embodiments, the methods and dispensing devices described herein are suitable for non-pharmaceutical applications, such as nutraceutics.

### XV. Embodiments Having and/or Resulting from a Combination of a Nano-Suspension and an ASD

Provided herein, in certain aspects, are embodiments having and/or resulting from a nano-suspension formulation, as described herein, and an amorphous solid dispersion (ASD) formulation, as described herein. For example, in some embodiments, provided herein is a method of making a dosage form, the method comprising depositing a plurality of droplets of a nano-suspension formulation described herein onto a substrate using a high frequency droplet dispenser, and depositing a plurality of droplets of an ASD formulation described herein onto the substrate using a high frequency droplet dispenser, thereby making the dosage form. In some embodiments, the devices, systems, and/or dispensing cartridges disclosed herein may comprise a nano-suspension formulation, as described herein, and an ASD formulation, as described herein. For example, in some embodiments, the device and/or system comprises a first dispensing cartridge comprising a nano-suspension formulation, as described herein, and a second dispensing cartridge comprising an ASD formulation, as described herein.

It is to be understood that in every embodiment comprising an amorphous solid dispersion (ASD), an embodiment is also envisioned that the ASD is a nanoparticle-forming ASD. Such nanoparticle-forming ASDs can, in some embodiments, be further defined by the teachings provided herein for nano-suspensions and products produced therefrom, e.g., particle size. In some embodiments, the nanosuspension is an ASD formulation (as herein described). In an embodiment, the ASD formulation is a nanoparticle-forming ASD (as herein described).

The present invention is not intended to be limited in scope to the particular disclosed embodiments, which are provided, for example, to illustrate various aspects of the invention. Various modifications to the compositions and methods described will become apparent from the description and teachings herein. Such variations may be practiced without departing from the true scope and spirit of the disclosure and are intended to fall within the scope of the present disclosure

### ENUMERATED EMBODIMENTS

Embodiment 1. A method of making a dosage form comprising a hydrophobic agent, the method comprising:
   depositing a plurality of droplets of a nano-suspension onto a substrate using a high frequency droplet dispenser to form a dosage form, wherein said nano-suspension comprises:
   an aqueous carrier, and
   a plurality of nanoparticles suspended in said aqueous carrier, wherein said nanoparticles comprise the hydrophobic agent, and
wherein the viscosity of the nano-suspension is less than 3 cP.
Embodiment 2. The method of embodiment 1, wherein the high frequency droplet dispenser deposits the plurality of droplets using a thermal process.
Embodiment 3. The method of embodiment 1, wherein the high frequency droplet dispenser deposits the plurality of droplets using a piezoelectric process.
Embodiment 4. The method of any one of embodiments 1-3, wherein the high frequency droplet dispenser comprises about 100 to about 1000 individual nozzles.
Embodiment 5. The method of embodiment 4, wherein each nozzle comprises an aperture with a diameter of about 20 μm to about 120 μm.
Embodiment 6. The method of any one of embodiments 1-5, wherein the high frequency droplet dispenser is a high frequency, sub-nanoliter droplet dispenser.
Embodiment 7. The method of any one of embodiments 1-6, wherein the high frequency droplet dispenser dispenses droplets with a volume of 1 nanoliter or less.
Embodiment 8. The method of embodiment 7, wherein the average volume of each dispensed droplet of the plurality of droplets of the nano-suspension is about 1 picoliter to about 50 picoliters.
Embodiment 9. The method of embodiment 7 or 8, wherein the plurality of nano-suspension droplets are deposited onto the substrate according to an average number of droplets per square inch.
Embodiment 10. The method of embodiment 9, wherein the average number of droplets deposited per square inch of the substrate is about 1 droplet to about 500 droplets per square inch.
Embodiment 11. The method of embodiment 9 or 10, wherein the plurality of droplets of the nano-suspension deposited on the substrate have an average diameter of about 5 μm to about 100 μm.
Embodiment 12. The method of any one of embodiments 1-11, wherein the plurality of droplets of the nano-suspension deposited on the substrate form a continuous layer.
Embodiment 13. The method of any one of embodiments 1-12, wherein the viscosity of the nano-suspension is less than 2 cP and greater than 1 cP.
Embodiment 14. The method of any one of embodiments 1-13, wherein the viscosity of the nano-suspension is between 1.2 cP to 1.8 cP.
Embodiment 15. The method of any one of embodiments 1-14, wherein the nano-suspension further comprises a viscosity-modulating agent.
Embodiment 16. The method of embodiment 15, wherein the viscosity-modulating agent is a viscosity-enhancing agent.
Embodiment 17. The method of embodiment 16, wherein the viscosity-enhancing agent is selected from the group consisting of ethylene glycol, polyethylene glycol, glycerol, and propylene glycerol.
Embodiment 18. The method of embodiment 16 or 17, wherein the viscosity-enhancing agent is present in the nano-suspension at a concentration of about 1% to about 40% v/v of the nano-suspension.
Embodiment 19. The method of any one of embodiments 16-18, wherein the ratio of the viscosity-enhancing agent to the aqueous carrier of the nano-suspension is from about 1:1000 to about 1:2 by volume.
Embodiment 20. The method of any one of embodiments 16-19, further comprising adding the viscosity-enhancing agent to the nano-suspension to achieve a viscosity of the nano-suspension of between about 1 cP and about 2 cP.
Embodiment 21. The method of any one of embodiments 1-20, wherein the aqueous carrier comprises water and an organic solvent.
Embodiment 22. The method of any one of embodiments 1-21, wherein the nano-suspension is a colloidal suspension.
Embodiment 23. The method of any one of embodiments 1-22, wherein the nano-suspension further comprises a stabilizing component.
Embodiment 24. The method of embodiment 23, wherein the ratio of the stabilizing component to the aqueous carrier of the nano-suspension is about 1:500 to about 1:10 by weight.
Embodiment 25. The method of embodiment 23, wherein the stabilizing component comprises PVP and/or SDS.
Embodiment 26. The method of any one of embodiments 1-25, wherein the average diameter of the plurality of nanoparticles is less than about 500 nm.
Embodiment 27. The method of any one of embodiments 1-26, wherein the plurality of nanoparticles has a D50 of about 100 nm to about 400 nm.
Embodiment 28. The method of any one of embodiments 1-27, wherein the plurality of nanoparticles has a D90 of about 250 μm to about 50 nm.
Embodiment 29. The method of any one of embodiments 1-28, wherein the hydrophobic agent is not soluble in the aqueous carrier.
Embodiment 30. The method of embodiment 29, wherein the hydrophobic agent has a solubility in water of less than 1 mg/mL.
Embodiment 31. The method of embodiment 29, wherein the hydrophobic agent is a therapeutic agent.
Embodiment 32. The method of embodiment 31, wherein the therapeutic agent is a small molecule drug.
Embodiment 33. The method of embodiment 31, wherein the therapeutic agent is a biomolecule.
Embodiment 34. The method of embodiment 31, wherein the therapeutic agent is a Biopharmaceutics Classification System (BCS) class II or class IV agent
Embodiment 35. The method of any one of embodiments 32-34, wherein the therapeutic agent has a narrow therapeutic index.
Embodiment 36. The method of any one of embodiments 32-35, wherein the therapeutic agent a high-potency drug.
Embodiment 37. The method of embodiment 29 or 30, wherein the hydrophobic agent is an excipient or an adjuvant.
Embodiment 38. The method of any one of embodiments 1-37, wherein the concentration of the hydrophobic agent in the nano-suspension is about 5 mg/mL to about 1,000 mg/mL.
Embodiment 39. The method of any one of embodiments 1-38, wherein the amount of the hydrophobic agent is less than 0.1 mg.
Embodiment 40. The method of any one of embodiments 1-39, wherein the substrate is a pharmaceutically acceptable polymer.
Embodiment 41. The method of any one of embodiments 1-40, wherein the substrate is selected from the group consisting of one or more of chitosan, ethylenediaminetetraacetic acid, polyvinyl pyrrolidone, polyvinyl alcohol, alginate, agar, carrageenan, guar gum, xanthan gum, polycarbophil, and polyacrylic acid derivatives.
Embodiment 42. The method of any one of embodiments 1-41, wherein the pharmaceutically acceptable substrate is a thin film substrate.
Embodiment 43. The method of any one of embodiments 1-42, wherein at least a portion of the plurality of nanoparticles is within the substrate.
Embodiment 44. The method of any one of embodiments 1-43, wherein at least a portion of the plurality of nanoparticles is on top of the substrate.
Embodiment 45. The method of any one of embodiments 1-44, wherein the dosage form is an oral dosage form.
Embodiment 46. The method of any one of embodiments 1-44, wherein the dosage form is a buccal dosage form.
Embodiment 47. The method of any one of embodiments 1-44, wherein the dosage form is a transdermal dosage form.
Embodiment 48. The method of any one of embodiments 1-47, wherein the dosage form is sterile.
Embodiment 49. A nano-suspension comprising:
   an aqueous carrier;
   a plurality of nanoparticles comprising a hydrophobic agent suspended in the aqueous carrier, wherein the viscosity of the nano-suspension is less than 3 cP.
Embodiment 50. The nano-suspension of embodiment 49, wherein the viscosity of the nano-suspension is less than 2 cP and greater than 1 cP.
Embodiment 51. The nano-suspension of embodiment 49 or 50, wherein the viscosity of the nano-suspension is between 1.2 cP to 1.8 cP.
Embodiment 52. The nano-suspension of any one of embodiments 49-51, wherein the nano-suspension further comprises a viscosity-modulating agent.
Embodiment 53. The nano-suspension of embodiment 52, wherein the viscosity-modulating agent is a viscosity-enhancing agent.
Embodiment 54. The nano-suspension of embodiment 53, wherein the viscosity-enhancing agent is selected from the group consisting of ethylene glycol, polyethylene glycol, glycerol, and propylene glycerol.
Embodiment 55. The nano-suspension of embodiment 52 or 53, wherein the viscosity-enhancing agent is present in the nano-suspension at a concentration of about 1% to about 40% v/v of the nano-suspension.
Embodiment 56. The nano-suspension of any one of embodiments 53-55, wherein the ratio of the viscosity enhancing agent to the aqueous carrier of the nano-suspension is about 1:1000 to about 1:2 by volume.
Embodiment 57. The nano-suspension of any one of embodiments 53-56, further comprising adding the viscosity-enhancing agent to the nano-suspension to achieve a viscosity of the nano-suspension of between about 1 cP and about 2 cP.
Embodiment 58. The nano-suspension of any one of embodiments 49-57, wherein the aqueous carrier comprises water and an organic solvent.
Embodiment 59. The nano-suspension of any one of embodiments 49-58, wherein the nano-suspension is a colloidal suspension.
Embodiment 60. The nano-suspension of any one of embodiments 49-59, wherein the nano-suspension further comprises a stabilizing component.
Embodiment 61. The nano-suspension of embodiment 60, wherein the ratio of the stabilizing component to the aqueous carrier of the nano-suspension is about 1:500 to about 1:10 by weight.
Embodiment 62. The nano-suspension of embodiment 60, wherein the stabilizing component comprises PVP and/or SDS.
Embodiment 63. The nano-suspension of any one of embodiments 49-62, wherein the average diameter of the plurality of nanoparticles is less than about 500 nm.
Embodiment 64. The nano-suspension of any one of embodiments 49-63, wherein the plurality of nanoparticles has a D50 of about 100 nm to about 400 nm.
Embodiment 65. The nano-suspension of any one of embodiments 49-64, wherein the plurality of nanoparticles has a D90 of about 250 μm to about 50 nm.
Embodiment 66. The nano-suspension of any one of embodiments 49-65, wherein the hydrophobic agent is not soluble in the aqueous carrier.
Embodiment 67. The nano-suspension of embodiment 66, wherein the hydrophobic agent has a solubility in water of less than 1 mg/mL.
Embodiment 68. The nano-suspension of embodiment 66, wherein the hydrophobic agent is a therapeutic agent.
Embodiment 69. The nano-suspension of embodiment 68, wherein the therapeutic agent is a small molecule drug.
Embodiment 70. The nano-suspension of embodiment 68, wherein the therapeutic agent is a biomolecule.
Embodiment 71. The nano-suspension of embodiment 66, wherein the hydrophobic agent is a Biopharmaceutics Classification System (BCS) class II or class IV agent.
Embodiment 72. The nano-suspension of any one of embodiments 69-71, wherein the therapeutic agent has a narrow therapeutic index.
Embodiment 73. The nano-suspension of any one of embodiments 67-72, wherein the therapeutic agent a high-potency drug.
Embodiment 74. The nano-suspension of embodiment 66 or 67, wherein the hydrophobic agent is an excipient or an adjuvant.
Embodiment 75. The nano-suspension of any one of embodiments 49-74, wherein the concentration of the hydrophobic agent in the nano-suspension is about 5 mg/mL to about 1,000 mg/mL.
Embodiment 76. The nano-suspension form of any one of embodiments 49-75, wherein the amount of the hydrophobic agent is less than 0.1 mg.
Embodiment 77. A composition comprising a dried or lyophilized nano-suspension, wherein upon reconstitution, the nano-suspension of any one of embodiments 49-76 is formed.
Embodiment 78. A dosage form prepared by the method of embodiment 1.
Embodiment 79. The dosage form of embodiment 78, wherein the dosage form comprises a fixed amount of the hydrophobic agent, wherein the hydrophobic agent is a therapeutic agent, and a fixed amount of a second hydrophobic agent.
Embodiment 80. The dosage form of embodiment 79, wherein the second hydrophobic agent is a therapeutic agent.
Embodiment 81. The dosage form of embodiment 79, wherein the second hydrophobic agent is a pharmaceutically acceptable excipient.
Embodiment 82. A plurality of the dosage form of any one of embodiments 78-81 prepared as a batch of drug dosage forms, wherein the amount of the hydrophobic agent of each drug dosage form of the batch does not vary more than 5% from the average.
Embodiment 83. A system for producing a dosage form, said system comprising:
   a nano-suspension comprising a hydrophobic agent, wherein the nano-suspension comprises an aqueous carrier and a plurality of nanoparticles, wherein each nanoparticle of the plurality of nanoparticles comprises the hydrophobic agent, and wherein the viscosity of the nano-suspension is less than 2 cP; and
   a high frequency droplet dispenser comprising about 100 to about 1000 individual nozzles, each nozzle configured to deposit at least one nano-suspension droplet onto a substrate of the dosage form.
Embodiment 84. The system of embodiment 83, wherein each nozzle has an aperture diameter of about 20 μm to about 120 μm.
Embodiment 85. The system of embodiment 83 or 84, wherein the nano-suspension droplet is deposited onto the substrate using a thermal process.
Embodiment 86. The system of embodiment 83 or 84, wherein the nano-suspension droplet is deposited onto the substrate using a piezoelectric process.

Second enumerated list of embodiments:
Embodiment 1A. A method of making a dosage form comprising an amorphous solid dispersion (ASD) comprising a hydrophobic agent, the method comprising depositing droplets of an ASD forming solution comprising the hydrophobic agent, an organic carrier, and a polymer onto a substrate using a droplet dispenser, wherein the ratio of the polymer and the hydrophobic agent inhibits crystallization of the hydrophobic agent in a dried state on the substrate.
Embodiment 2A. The method of embodiment 1A, wherein the droplet dispenser dispenses the plurality of droplets using a thermal process.
Embodiment 3A. The method of embodiment 1A, wherein the droplet dispenser dispenses the plurality of droplets using a piezoelectric process.
Embodiment 4A. The method of any one of embodiments 1A-3A, wherein the droplet dispenser comprises about 100 to about 1000 individual nozzles.
Embodiment 5A. The method of embodiment 4A, wherein each nozzle comprises an aperture with a diameter of about 20 μm to about 120 μm.
Embodiment 6A. The method of any one of embodiments 1A-5A, wherein the droplet dispenser is a sub-nanoliter droplet dispenser.
Embodiment 7A. The method of embodiment 6A, wherein the sub-nanoliter droplet dispenser is a high frequency, sub-nanoliter droplet dispenser.
Embodiment 8A. The method of embodiment 7A, wherein the high frequency, sub-nanoliter droplet dispenser dispenses droplets at more than 3 kHz as measured per nozzle.
Embodiment 9A. The method of any one of embodiments 1A-8A, wherein the droplet dispenser dispenses droplets with a volume of 1 nanoliter or less.
Embodiment 10A. The method of embodiment 9A, wherein the average volume of dispensed droplets of the ASD forming solution is about 1 picoliter to about 50 picoliters.
Embodiment 11A. The method of any one of embodiments 1A-10A, wherein the droplets of the ASD forming solution are deposited onto the substrate according to an average number of droplets per square inch.
Embodiment 12A. The method of embodiment 11A, wherein the average number of droplets of the ASD forming solution deposited per square inch of the substrate is about 1 droplet to about 500 droplets per square inch.
Embodiment 13A. The method of any one of embodiments 9A-12A, wherein the droplets of the ASD forming solution dispensed from the droplet dispenser have an average largest crossing dimension of about 5 μm to about 100 μm.
Embodiment 14A. The method of any one of embodiments 1A-13A, wherein the droplets of the ASD forming solution deposited on the substrate form a continuous layer.
Embodiment 15A. The method of any one of embodiments 1A-14A, wherein the ASD forming solution has a viscosity of 0.5 cP to 3 cP.
Embodiment 16A. The method of any one of embodiments 1A-15A, wherein the ASD forming solution has a viscosity of between 1 cP to 2 cP.
Embodiment 17A. The method of any one of embodiments 1A-16A, wherein the ASD forming solution has a viscosity of 1.2 cP to about 1.8 cP.
Embodiment 18A. The method of any one of embodiments 1A-17A, wherein the ASD forming solution further comprises a viscosity-modulating agent.
Embodiment 19A. The method of embodiment 18A, wherein the viscosity-modulating agent is a viscosity-enhancing agent.
Embodiment 20A. The method of embodiment 19A, wherein the viscosity-enhancing agent is ethylene glycol, polyethylene glycol, glycerol, or propylene glycerol, or any combination thereof.
Embodiment 21A. The method of embodiment 18A, wherein the viscosity-modulating agent is a viscosity-reducing agent.
Embodiment 22A. The method of embodiment 21A, wherein the viscosity-reducing agent is a salt or an amino acid, or a combination thereof.
Embodiment 23A. The method of any one of embodiments 18A-22A, wherein the concentration of the viscosity-modulating agent in the ASD forming solution is 1% to 40%.
Embodiment 24A. The method of any one of embodiments 18A-23A, wherein the viscosity-modulating agent has a ratio to the organic carrier of 1:100 to 40:100 by volume.
Embodiment 25A. The method of any one of embodiments 18A-24A, wherein the amount of the viscosity-modulating agent in the mixture is based on a desired viscosity of the mixture.
Embodiment 26A. The method of any one of embodiments 1A-25A, wherein the organic carrier of the ASD forming solution comprises any one or more of acetone, dimethylformamide, ethanol, methanol, pentane, hexane, heptane, ethyl acetate, isopropyl alcohol, ethylene glycol, and dimethyl sulfoxide.
Embodiment 27A. The method of any one of embodiments 1A-26A, wherein the ASD forming solution comprises at least 30% organic carrier by volume.
Embodiment 28A. The method of any one of embodiments 1A-27A, wherein the polymer of the ASD forming solution is suitable for formation of an amorphous solid dispersion (ASD) following deposition by the droplet dispenser.
Embodiment 29A. The method of any one of embodiments 1A-28A, wherein the polymer is selected based on one or more characteristics of the hydrophobic agent.
Embodiment 30A. The method of any one of embodiments 1A-29A, wherein the polymer is selected from the group consisting of a cellulose derivative, poly(vinyl pyrrolidinone) (PVP), poly(vinyl pyrrolidinone-co-vinyl acetate) (copovidone, or PVPVA), hydroxypropyl methyl cellulose (hypromellose, or HPMC), and HPMC acetate succinate (hypromellose acetate succinate or HPMC-AS), and eudragit, or any combination thereof.
Embodiment 31A. The method of any one of embodiments 1A-30A, wherein the polymer is a grade selected from the group consisting of LF, MF, HF, LMP, MMP, HMP, LG, MG, and HG.
Embodiment 32A. The method of any one of embodiments 1A-31A, wherein the polymer is HPMC-AS-LF.
Embodiment 33A. The method of any one of embodiments 1A-32A, wherein the ratio of the concentration of the polymer to the concentration of the drug in the ASD forming solution is from 1:20 to 20:1.
Embodiment 34A. The method of any one of embodiments 1A-33A, wherein ratio of the concentration of the polymer to the concentration of the drug in the ASD forming solution is 1:2 to 2:1.
Embodiment 35A. The method of any one of embodiments 1A-34A, wherein less than 5% of the hydrophobic agent crystallizes in a dried state following deposition by the droplet dispenser.
Embodiment 36A. The method of any one of embodiments 1A-35A, wherein substantially no crystallization of the hydrophobic agent occurs in a dried state.
Embodiment 37A. The method of any one of embodiments 1A-36A, wherein the ASD forming solution is supersaturated with the hydrophobic agent.
Embodiment 38A. The method of any one of embodiments 1A-37A, wherein the hydrophobic agent is a drug.
Embodiment 39A. The method of embodiment 38A, wherein the drug is a small molecule drug.
Embodiment 40A. The method of embodiment 38A or 39A, wherein the agent is a Biopharmaceutics Classification System (BCS) class II or IV drug.
Embodiment 41A. The method of embodiment 38A, wherein the drug is a biomolecule.
Embodiment 42A. The method of embodiment 41A, wherein the biomolecule is a polypeptide.
Embodiment 43A. The method of any one of embodiments 38A-42A, wherein the drug has a narrow therapeutic index.
Embodiment 44A. The method of any one of embodiments 38A-43A, wherein the drug a high-potency drug.
Embodiment 45A. The method of any one of embodiments 1A-37A, wherein the hydrophobic agent is a diagnostic compound.
Embodiment 46A. The method of any one of embodiments 1A-37A, wherein the hydrophobic agent is a reagent.
Embodiment 47A. The method of any one of embodiments 1A-37A, wherein the hydrophobic agent is an adjuvant or an excipient.
Embodiment 48A. The method of any one of embodiments 1A-47A, wherein the agent has a solubility in water of less than 0.1 mg/mL.
Embodiment 49A. The method of any one of embodiments 1A-48A, wherein the concentration of the agent in the mixture is about 5 mg/mL to about 1,000 mg/mL.
Embodiment 50A. The method of any one of embodiments 1A-49A, wherein the ASD forming solution further comprises a surfactant.
Embodiment 51A. The method of embodiment 50A, wherein the surfactant is selected from Tween 80, Vit-E, TPGS, or SDS, or any combination thereof.
Embodiment 52A. The method of embodiment 50A or 51A, wherein the ASD forming solution comprises 10% to 20% of the surfactant by volume.
Embodiment 53A. The method of any one of embodiments 50A-52A, wherein the ASD forming solution comprises a ratio of the surfactant to the hydrophobic agent of 1:100 to 5:1.
Embodiment 54A. The method of any one of embodiments 1A-53A, wherein the substrate is a pharmaceutically acceptable polymer.
Embodiment 55A. The method of any one of embodiments 1A-54A, wherein the substrate comprises chitosan, ethylenediaminetetraacetic acid, polyvinyl pyrrolidone, polyvinyl alcohol, alginate, agar, carrageenan, guar gum, xanthan gum, polycarbophil, or polyacrylic acid derivatives, or any combination thereof.
Embodiment 56A. The method of any one of embodiments 1A-55A, wherein the substrate is a thin film substrate.
Embodiment 57A. The method of any one of embodiments 1A-56A, wherein at least a portion of the hydrophobic agent is within the substrate following deposition by the droplet dispenser.
Embodiment 58A. The method of any one of embodiments 1A-57A, wherein at least a portion of the hydrophobic agent is on top of the substrate following deposition by the droplet dispenser.
Embodiment 59A. The method of any one of embodiments 1A-58A, wherein the dosage form is an oral dosage form.
Embodiment 60A. The method of any one of embodiments 1A-58A, wherein the dosage form is a buccal dosage form.
Embodiment 61A. The method of any one of embodiments 1A-58A, wherein the dosage form is a transdermal dosage form.
Embodiment 62A. The method of any one of embodiments 1A-61A, wherein the dosage form is sterile.
Embodiment 63A. The method of any one of embodiments 1A-62A, wherein the ASD forming solution is a colloid.
Embodiment 64A. The method of any one of embodiments 1A-63A, further comprising making the ASD forming solution.
Embodiment 65A. An ASD forming solution comprising:
   an organic carrier,
   a hydrophobic agent, and
   a polymer crystallization inhibitor,
      wherein the polymer inhibits crystallization of the agent in a dried state.
Embodiment 66A. The ASD formulation solution of embodiment 65A, wherein the ASD forming solution has a viscosity of 0.5 cP to 3 cP.
Embodiment 67A. The ASD forming solution of any one of embodiments 65A-66A, wherein the ASD forming solution has a viscosity of between 1 cP to 2 cP.
Embodiment 68A. The ASD forming solution of any one of embodiments 65A-67A, wherein the ASD forming solution has a viscosity of 1.2 cP to about 1.8 cP.
Embodiment 69A. The ASD forming solution of any one of embodiments 65A-68A, wherein the ASD forming solution comprises a viscosity modulating agent.
Embodiment 70A. The ASD forming solution of any one of embodiments 65A-68A, wherein the viscosity-modulating agent is a viscosity-enhancing agent.
Embodiment 71A. The ASD forming solution of any one of embodiments 65A-70A, wherein the viscosity-enhancing agent is ethylene glycol, polyethylene glycol, glycerol, or propylene glycerol, or any combination thereof.
Embodiment 72A. The ASD forming solution of any one of embodiments 65A-69A, wherein the viscosity-modulating agent is a viscosity-reducing agent.
Embodiment 73A. The ASD forming solution of any one of embodiments 65A-69A or 72A, wherein the viscosity-reducing agent is a salt or an amino acid, or a combination thereof.
Embodiment 74A. The ASD forming solution of any one of embodiments 65A-73A, wherein the concentration of the viscosity-modulating agent in the ASD forming solution is 1% to 40%.
Embodiment 75A. The ASD forming solution of any one of embodiments 65A-74A, wherein the viscosity-modulating agent has a ratio to the organic carrier of 1:100 to 40:100 by volume.
Embodiment 76A. The ASD forming solution of any one of embodiments 6A5-75A, wherein the amount of viscosity-modulating agent in the mixture is based on a desired viscosity of the mixture.
Embodiment 77A. The ASD forming solution of any one of embodiments 65A-76A, wherein the organic carrier of the ASD forming solution comprises any one or more of acetone, dimethylformamide, ethanol, methanol, pentane, hexane, heptane, ethyl acetate, isopropyl alcohol, ethylene glycol, and dimethyl sulfoxide.
Embodiment 78A. The ASD forming solution of any one of embodiments 65A-77A, wherein the ASD forming solution comprises at least 30% organic carrier by volume.
Embodiment 79A. The ASD forming solution of any one of embodiments 65A-78A, wherein the polymer is selected based on one or more characteristics of the hydrophobic agent.
Embodiment 80A. The ASD forming solution of any one of embodiments 65A-79A, wherein the polymer is selected from the group consisting of a cellulose derivative, poly (vinyl pyrrolidinone) (PVP), poly(vinyl pyrrolidinone-co-vinyl acetate) (copovidone, or PVPVA), hydroxypropyl methyl cellulose (hypromellose, or HPMC), and HPMC acetate succinate (hypromellose acetate succinate or HPMC-AS), and eudragit, or any combination thereof.
Embodiment 81A. The ASD forming solution of any one of embodiments 65A-80A, wherein the polymer is a grade selected from the group consisting of LF, MF, HF, LMP, MMP, HMP, LG, MG, and HG.
Embodiment 82A. The ASD forming solution of any one of embodiments 65A-81A, wherein the polymer is HPMC-AS-LF.
Embodiment 83A. The ASD forming solution of any one of embodiments 65A-82A, wherein the ratio of the concentration of the polymer to the concentration of the drug in the ASD forming solution is from 1:20 to 20:1
Embodiment 84A. The ASD forming solution of any one of embodiments 65A-83A, wherein the ratio of the concentration of the polymer to the concentration of the drug in the ASD forming solution is 1:2 to 2:1.
Embodiment 85A. A dispensing cartridge comprising the ASD forming solution of any one of embodiments 61A-84A.
Embodiment 86A. A dosage form comprising an amorphous solid dispersion of a hydrophobic agent deposited on a substrate via droplets.
Embodiment 87A. A dosage form prepared by the method of any one of embodiments 1A-64A.
Embodiment 88A. The dosage form of embodiment 87A, wherein the dosage form comprises a fixed amount of the hydrophobic agent, wherein the hydrophobic agent is a therapeutic agent, and a fixed amount of a second hydrophobic agent.
Embodiment 89A. The dosage form of embodiment 87A, wherein the second hydrophobic agent is a therapeutic agent.
Embodiment 90A. The dosage form of embodiment 87A, wherein the second hydrophobic agent is a pharmaceutically acceptable excipient.
Embodiment 91A. A method of determining a ratio of a hydrophobic agent and a polymer that forms an amorphous solid dispersion, the method comprising dispensing onto a substrate an amount of a first precursor solution comprising a first organic carrier, the hydrophobic agent, and a first viscosity-enhancing agent, and an amount of a second precursor solution comprising a second organic carrier, a polymer, and a second viscosity-enhancing agent to determine the ratio of the hydrophobic agent and the polymer that forms an amorphous solid dispersion of the hydrophobic agent and the polymer, wherein the dispensing causes the first precursor solution to contact the second precursor solution.
Embodiment 92A. The method of embodiment 91A, wherein the dispensing mixes the first precursor solution and the second precursor solution on the substrate.
Embodiment 93A. The method of embodiment 91A, wherein the dispensing mixes the first precursor solution and the second precursor solution prior to being applied to the substrate.
Embodiment 94A. The method of any one of embodiments 91A-93A, wherein the amount of the first precursor solution and the amount of the second precursor solution are dispensed such that a gradient of ratios of the agent and the polymer are formed in an area on the substrate.
Embodiment 95A. The method of embodiment 94A, wherein the amount of the first precursor solution and the amount of the second precursor solution are dispensed such that an area on the substrate has a pre-determined ratio of the agent and the polymer.
Embodiment 96A. The method of any one of embodiments 91A-95A, wherein the amount of the first precursor solution is dispensed from a first sub-nanoliter droplet dispenser, and wherein the amount of the second precursor solution is dispensed from a second sub-nanoliter droplet dispenser.
Embodiment 97A. The method of any one of embodiments 91A-96A, further comprising assessing the crystallization of the agent.
Embodiment 98A. The method of any one of embodiments 91A-97A, wherein the first organic carrier and the second organic carrier are the same.
Embodiment 99A. The method of any one of embodiments 91A-98A, wherein the first viscosity-enhancing agent and the second viscosity-enhancing agent are the same.
Embodiment 100A. The method of any one of embodiments 91A-99A, wherein the substrate is a thin film.
Embodiment 101A. A droplet dispenser for producing a dosage form comprising an amorphous solid dispersion of a hydrophobic agent, the dispenser comprising:
   (a) a dispensing head comprising one or more nozzles;
   (b) an ASD forming solution comprising an organic carrier, the agent, a polymer, and a viscosity-modulating agent,
      wherein the agent and the polymer are present in the mixture in a ratio such that crystallization of the agent is inhibited in a dried state on a substrate,
      wherein the mixture cartridge is operably coupled to the dispensing head; and
   (c) a control system configured to dispense the mixture via the dispensing head according to dispensing pattern.
Embodiment 102A. The dispenser of embodiment 101A, wherein the dispensing head comprises about 100 to about 1000 nozzles.
Embodiment 103A. The dispenser of embodiment 101A or 102A, wherein each nozzle has an aperture diameter of about 20 μm to about 120 μm.
Embodiment 104A. The dispenser of any one of embodiments 101A-103A, wherein the dispensing head dispenses droplets using a thermal process.
Embodiment 105A. The dispenser of any one of embodiments 101A-104A, wherein the dispensing head dispenses droplets using a piezoelectric process.
Embodiment 106A. The dispenser of any one of embodiments 101A-105A, further comprising a second dispensing head operably coupled to a second mixture cartridge, wherein the second mixture cartridge comprises a second mixture comprising a second agent.
Embodiment 107A. The dispenser of embodiments 106A, further comprising a third dispensing head operably coupled to a third mixture cartridge, wherein the third mixture cartridge comprises a protective coating mixture.
Embodiment 108A. The dispenser of embodiment 107A, further comprising a fourth dispensing head operably coupled to a fourth dispensing cartridge, wherein the fourth mixture cartridge comprises an edible mixture.
Embodiment 109A. A gradient of two components on a substrate, comprising
   a first component comprising a hydrophobic agent;
   a second component comprising a polymeric crystallization inhibitor; and
   a ratio of the first component to the second component, wherein the ratio changes across the gradient.
Embodiment 110A. The gradient of embodiment 109A, wherein the first component further comprises an organic carrier.
Embodiment 111A. The gradient of embodiment 109A or 110A, wherein the first component further comprises a viscosity-enhancing agent.
Embodiment 112A. The gradient of any one of embodiments 109A-111A, wherein the second component further comprises an organic carrier.
Embodiment 113A. The gradient of any one of embodiments 109A-112A, wherein the second component further comprises a viscosity-enhancing agent.
Embodiment 114A. The gradient of any one of embodiments 109A-113A, wherein the second component is capable of inhibiting crystallization of the hydrophobic agent.
Embodiment 115A. The gradient of embodiment 109A, wherein the first and second components each comprise an organic carrier, wherein the organic carriers are the same.
Embodiment 116A. The gradient of embodiment 109A, wherein the first and second components each comprise a viscosity-enhancing agent, wherein the viscosity-enhancing agents are the same.
Embodiment 117A. The gradient of any one of embodiments 109A-116A, wherein the ratio becomes low enough at a certain point on the gradient to prevent crystallization of the hydrophobic agent.
Embodiment 118A. The gradient of any one of embodiments 109A-117A, wherein the first and second components are dispensed onto the substrate from a sub-nanoliter droplet dispenser.
Embodiment 119A. The gradient of any one of embodiments 109A-117A, wherein the first component is dispensed onto the substrate from a first sub-nanoliter droplet dispenser and the second is dispensed onto the substrate from a second sub-nanoliter droplet dispenser.
Embodiment 120A. The gradient of any one of embodiments 109A-119A, wherein the ratio changes at a consistent rate across the gradient.

### EXAMPLES

The following examples are included for illustrative purposes and are not intended to limit the scope of the teachings provided in the present application. Reagents and materials were obtained from commercial sources and use as received unless otherwise noted. Loadings and concentrations are reported as weight percent (wt. %) unless otherwise noted.

### Example 1: Production of a Nano-Suspension

Naproxen was used as received commercially. A 20-mL glass scintillation vial was charged with 45.6 grams of yttria-stabilized zirconia powder milling beads. Naproxen solid (0.876 g) was weighed into the vial. In a separate 20-mL vial, water (2.628 mL) and a stock solution of 5% polyvinylpyrrolidone (PVP, 4.380 mL) and 1% sodium dodecyl sulfate (SDS, 0.876 mL) was mixed. The aqueous solutions were then transferred to the vial containing naproxen to form a slurry at a naproxen:PVP:SDS ratio of 1:0.25:0.01 and an overall drug concentration of 100 mg/mL. The vial was sealed with a polytetrafluoroethylene cap and then placed on a Resonant Acoustic Mixer to mill at an intensity of 40 G's for 2 hours.

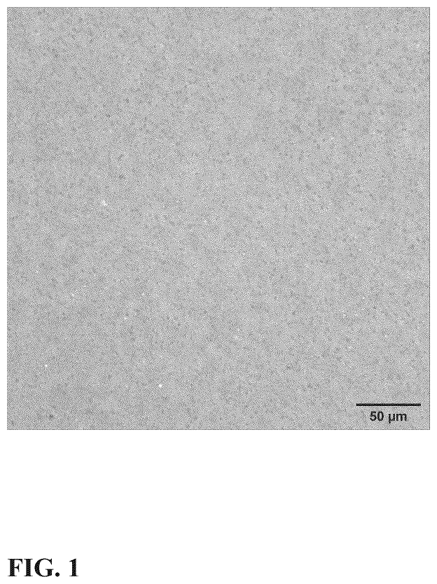

The resulting liquid nano-suspension sample was removed from the milling beads by extraction with a syringe equipped with a 26 gauge needle, giving an average yield of approximately 7 grams (80%). The resulting nano-suspension appeared as a white, opaque mixture with well-dispersed and suspended nanoparticles only visible under high magnification with optical microscopy (see FIG. 1). The nano-suspension at 100 mg/mL drug concentration was diluted with the appropriate volume of water to afford formulation samples at 10 mg/mL and 1 mg/mL drug concentration. Ethylene glycol was added as a viscosity-enhancing agent to some formulation samples.

### Example 2: Viscosity Measurements and Inkjet Printing of Nano-Suspension

The viscosity of the naproxen formulations was measured using a viscometer. A recirculating water bath was used to maintain a temperature of 25° C. at the sample chamber. An aliquot of the formulation sample (0.5 mL) was added to the sample chamber and the viscosity was recorded after the measurements had stabilized to <0.1% variation.

A commercial Inkjet Photo Printer equipped with a standard thermal inkjet print head was used to print patterns of the nano-suspension. Refillable ink cartridges were used as received and loaded with the liquid formulations for printing. The nano-suspensions were introduced to the cartridge via an aperture at the top to fill the internal reservoir. The cartridges had a spongy material used to help control the rate of release of the liquid through a large aperture at the bottom of the cartridge, where it entered the inkjet print head for final dispensing. Adobe Photoshop was used to create a simple printing pattern consisting of a circle with a diameter of 100 pixels and with a resolution of 72 dots per inch (dpi) using an ink color from a single cartridge source (e.g., cyan). Commercial 8.5″×11″ acetate transparency film was used as the printing substrate. The printed output was allowed to air dry for several hours before analysis. Between printing runs, the inkjet print head was carefully washed with deionized water and acetone to remove any residual formulation. Results for four formulation samples are shown in Table 2.

The 1 and 10 mg/mL nano-suspension formulations were not retained in the cartridge and thus could not be printed effectively. The 10 mg/mL formulation with 10% ethylene glycol had viscosity of about 1.5 cP. This higher viscosity formulation was superior for printing, consistent with measurements of commercially available inks, which had viscosities between ˜1-5 cP. Further measurements were carried out to determine the effects of ethylene glycol on the stability of the formulations.

TABLE 1
Amenability of nano-suspension formulations of varying
concentration and viscosity for inkjet printing.

| Nano-suspension | Viscosity | |
|---|---|---|
| Concentration | (cP) | Printable? |
| 100 mg/mL | 2.146 | Yes |
| 10 mg/mL | 1.022 | No |
| 1 mg/mL | 0.958 | No |
| 10 mg/mL + 10% | 1.548 | Yes |
| ethylene glycol | | |

### Example 3: Particle Size Analysis Using Dynamic Light Scattering of Nano-Suspension

The particle size distribution of the naproxen formulations as prepared in Example 1 was determined. A 10 pL aliquot of each nano-suspension sample was taken and diluted in 90 μL of water for analysis using a dynamic light scattering instrument. A 40 pL aliquot of the diluted suspension was dispensed into a low volume black polystyrene 384-well plate for analysis. The particle size of each sample was reported as an average of 10 acquisitions with an acquisition time of 3 sec at 25° C. Autocorrelation curves were fit using either the cumulants or regularization method, and the D50 radius measurement was obtained and reported. The normalized polydispersity (% Pd) was calculated as the polydispersity divided by the estimated hydrodynamic radius from the cumulants fit of the autocorrelation function multiplied by 100. Results of the D50 radius are shown in Table 2. Adding ethylene glycol to the formulations increased the size of the nanoparticles in the nano-suspensions.

TABLE 2
Effect of addition of ethylene glycol as a viscosity
enhancing agent on nanoparticle size.

| Nano-suspension | |
|---|---|
| Composition | Radius (nm) |
| 10 mg/mL naproxen | 111 |
| 10 mg/mL naproxen + 10% ethylene glycol | 134 |
| 10 mg/mL naproxen + 20% ethylene glycol | 167 |

### Example 4: Drug Concentration Analysis of Printed Pattern with High Performance Liquid Chromatography

Drug concentration analyses were performed on the printed nano-suspension pattern from Example 2 using a high performance liquid chromatography (HPLC) system equipped with a quaternary pump, a photodiode array UV-Vis detector, and a reversed-phase alkylsilane column (3.5 μm particle size, 50×4.6 mm dimension). The printed patterns from Example 2 were cut and were then washed and fully dissolved with 1:1 acetonitrile:water. The resulting solutions were then analyzed by HPLC with drug concentrations compared to standards of known concentration. The mobile phases of the HPLC process consisted of 0.05% trifluoroacetic acid in water and in acetonotrile. A gradient elution was utilized with the acetonitrile mobile phase increasing from 2 to 65% over 6.5 min at a flow rate of 1.25 mL/min. The column was heated at 30° C. and the detection was carried out at 254 nm. Analysis of multiple printed patterns (n=3) indicated that the deposition of microgram quantities of drug onto the transparency film was highly reproducible with low variability. Results are shown in Table 3.

TABLE 3
Deposition analysis of printed samples of naproxen
nano-suspensions at 100 and 10 mg/mL concentrations

| Ink Formulation | Deposition |
|---|---|
| Concentration | (μg/cm²) |
| 100 mg/mL naproxen | 13.19 ± 0.28 |
| 10 mg/mL naproxen + 10% ethylene glycol | 1.22 ± 0.04 |

For the naproxen nano-suspension formulation with a drug concentration of 100 mg/mL, given the measured deposition of 13.19 pg/cm² and the target print resolution of 72 dpi in the pattern, a droplet size of 16.6 picoliters was calculated. This is well within the expected range for droplet sizes dispensed by similar inkjet print heads. This also indicates that there was no major disruption of droplet formation nor significant loss or degradation of the drug formulation during the printing process, consistent with the HPLC analytical results.

These results are consistent with expectations of the inkjet printing process as a precise method for controlling droplet deposition, demonstrating that no significant issues occurred despite replacing traditional printer ink with a drug formulation “ink” and enabling the manufacture of a consistent dosage form with good content uniformity. Moreover, the amount of drug deposited was directly proportional to the initial concentration of the nano-suspension ink. Reducing the initial concentration from 100 mg/mL to 10 mg/mL resulted in a 10-fold reduction in the amount of naproxen deposited onto the transparency film. In addition, HPLC analysis showed no presence of degradants or other impurities, suggesting that the drug remained stable throughout the inkjet printing process, despite the flash heating required for deposition.

### Example 5: Optical Analysis and Reconstitution of Printed Patterns

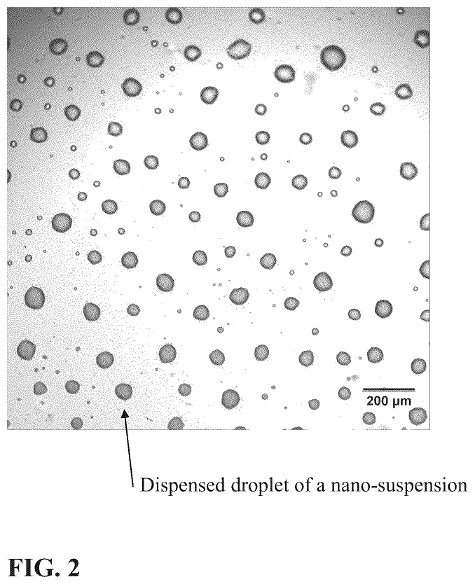

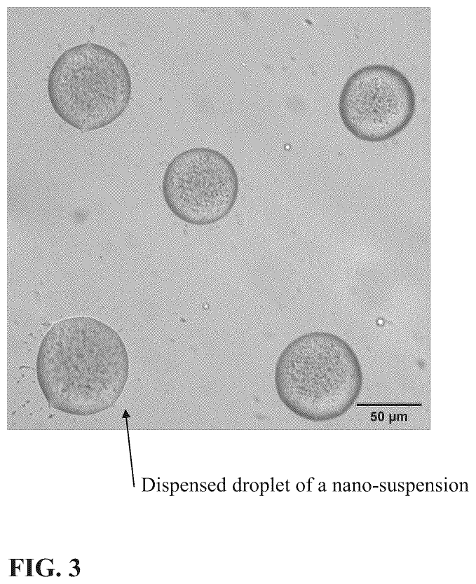

Optical microscopy analysis on the printed nano-suspension patterns from Example 2 was conducted using a microscope equipped with a camera. Commercially available acetate transparencies used for ink jet printing the nano-suspensions formulations were directly analyzed by brightfield transmitted light microscopy at various magnification scales. Results are shown in FIG. 2 and FIG. 3.

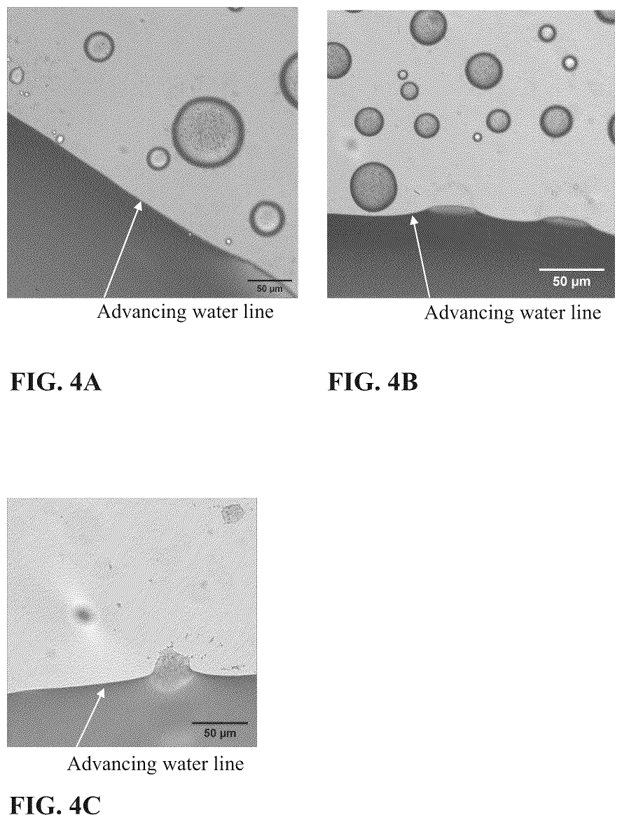

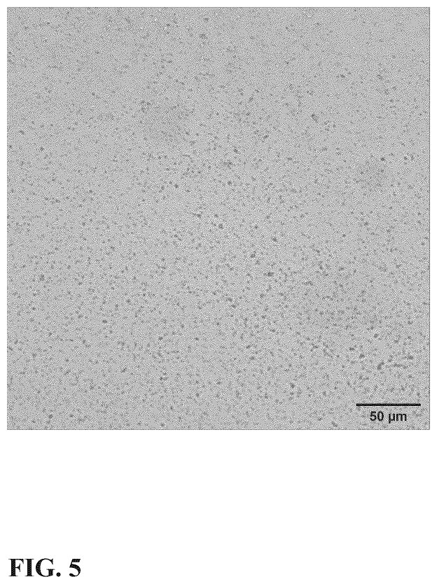

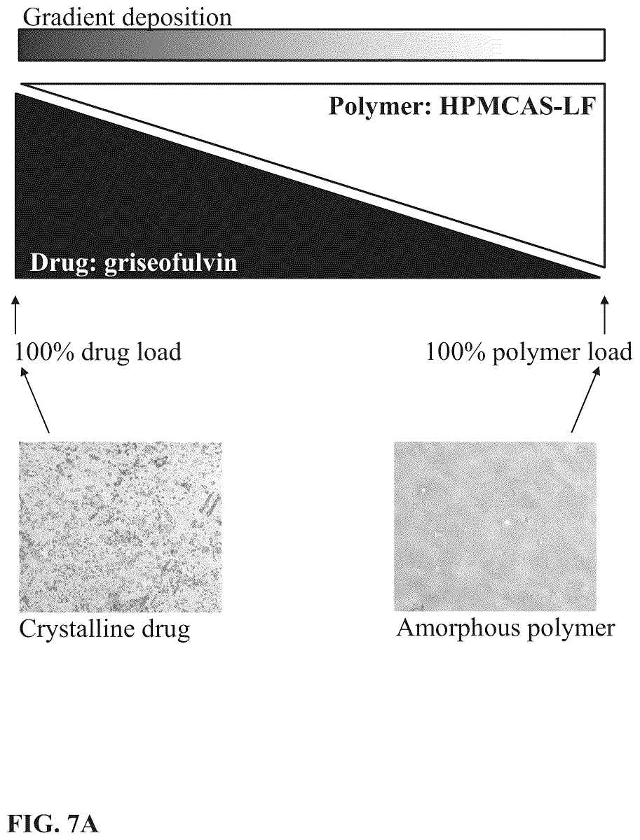

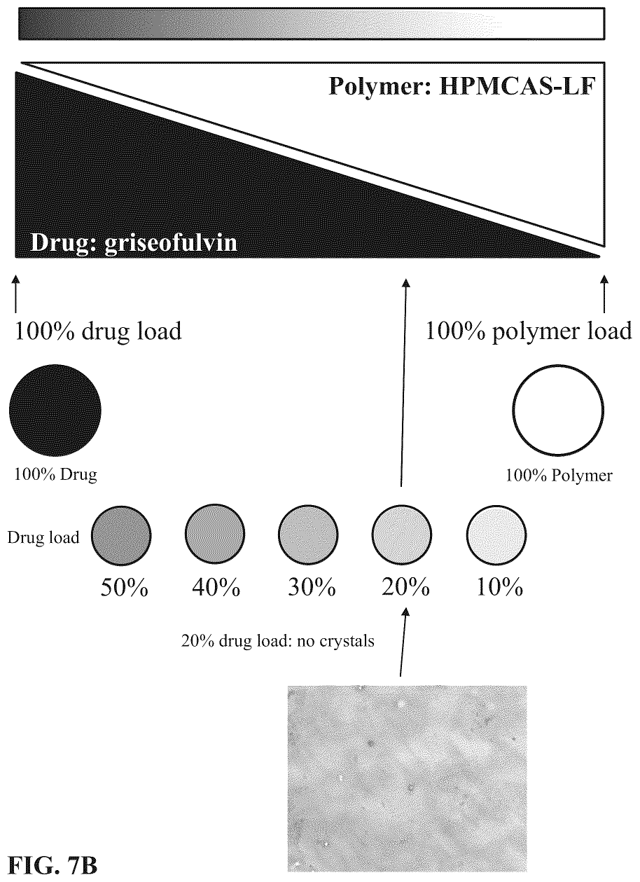

The reconstitution of the printed nano-suspensions after drying was then investigated. A drop of water was placed onto the transparency holding the dried and printed nano-suspensions from Example 3. The wetting and redispersion of the nano-suspension droplets was observed in real time by monitoring the advancing water line with optical microscopy (see FIGS. 4A-4C). Upon exposure to water, the dried nano-suspension droplets were immediately hydrated, releasing the drug nanoparticles into solution. The resulting reconstituted nano-suspension was recovered and analyzed by optical microscopy, showing a homogeneous, well-dispersed suspension with no significant aggregation (see FIG. 7). Dynamic light scattering analysis of the reconstituted sample showed particles consisting of 114 nm in diameter, showing no significant aggregation after drying and redispersion, confirming that the stability of the nano-suspension during this process was maintained.

### Example 6: Dispensing and Depositing of Organic Carriers with an Inkjet Printer

This example demonstrates dispensing and depositing of organic carriers using a droplet dispenser that dispenses droplets using a thermal process, thereby enabling dispensing of agents susceptible to crystallization, such as hydrophobic agents, and polymers for the production of amorphous solid dispersions comprising the agent.

Solvent mixtures were made according to Table 4. Refillable inkjet printer cartridges were used as received and loaded with the solvent mixtures for printing. A commercial Inkjet Photo Printer equipped with a standard thermal inkjet print head was used to dispensing and deposit droplets of the solvent mixtures onto a thin film substrate.

TABLE 4
Solvent mixtures and characteristics thereof.

| Solvent mixture | Viscosity (cP) | Printable? |
|---|---|---|
| 20% ethylene glycol in acetone mixture | 0.611 | No |
| 30% ethylene glycol in acetone mixture | 0.963 | No |
| 40% ethylene glycol in acetone mixture | 1.658 | Yes |
| 50% ethylene glycol in acetone mixture | 3.085 | No |

It was observed that if the solvent mixture was too fluid (low viscosity), then the solvent mixture leaked in certain droplet dispensers and from certain dispenser cartridges. On the other hand, if the solvent mixture was too viscous, then the dispensing of the solvent mixture was not possible or not reliable. The 40% ethylene glycol in acetone mixture having a 1.659 cp viscosity was successfully dispensed and deposited on the thin film substrate, thereby demonstrating that high concentration organic carrier are suitable solutions for use in droplet dispensers.

### Example 7: Production of an Amorphous Solid Dispersion Using an Inkjet Printer

This example demonstrates the identification of an ASD forming solution using two precursor solutions, and production and optimization of an ASD therefrom using droplet dispensers.

A first precursor solution was made comprising 2% griseofulvin (a fast crystallizer model drug; 20 mg/mL), an acetone organic carrier, and ethylene glycol. Various amounts of ethylene glycol, a viscosity-modulating agent, were assessed, including 30%, 35%, and 40%. Only the 35% and 40% ethylene glycol precursor solutions had a viscosity between 1 cp and 2 cp (respectively, 1.200 and 1.702), and the 40% ethylene glycol precursor solution was used to evaluate droplet dispensing.

A second precursor solution was made comprising 2% HPMCAS-LF (a polymer; 20 mg/mL) in an acetone organic carrier, and, optionally, with ethylene glycol. Various amounts of ethylene glycol, a viscosity-modulating agent, were assessed, including 0% (pure acetone), 5%, 20%, 30%, and 40%. Only the 0% and 5% ethylene glycol precursor solutions had a viscosity between 1 cp and 2 cp (respectively, 1.208 and 1.365), and the 5% ethylene glycol precursor solution was used to evaluate droplet dispensing. It is noted that the polymer, HPMCAS-LF, also serves to modulate viscosity of the precursor solution.

The first and second precursor solutions were added to independent refillable inkjet printer cartridges. A commercial Inkjet Photo Printer equipped with a standard thermal inkjet print head was used to dispensing and deposit droplets of the first and second precursor solutions onto a substrate according to desired ratios of griseofulvin and HPMCAS-LF, and methods described herein. Specifically, as shown in FIG. 7A, a gradient deposition was produced using two droplet dispensers (print heads). The amount of the first precursor solution dispensed from a first droplet dispenser and the amount of the second precursor solution dispensed from a second droplet dispenser can be varied across an area such that a gradient deposition is produced. The gradient can be varied as desired, and in this example a uniform gradient was produced, wherein on the left side of the gradient deposition the deposition comprises 100% load of griseofulvin and the right side of the gradient deposition the deposition comprises 100% HPMCAS-LF. Images were captured and show crystallization of griseofulvin at 100% load of griseofulvin, and the amorphous polymer at 100% load of HPMCAS-LF. Additionally, as shown in FIG. 7B, areas with a set load of griseofulvin were produced (100%, 50%, 40%, 30%, 20%, 10%, and 0%) relative to HPMCAS-LF. Captured images of the area with 20% load of griseofulvin as well as the corresponding 20% griseofulvin load on the gradient deposition demonstrated that an ASD of griseofulvin was produced using the droplet dispensing techniques taught herein. 20% griseofulvin load is known to produce an ASD via conventional large scale spray drying techniques. The results demonstrated herein show that the droplet dispenser technique is able to reproduce the known ASD properties on a miniaturized scale.

1. A method of making a dosage form comprising a hydrophobic agent, the method comprising:
depositing a plurality of droplets of a formulation comprising the hydrophobic agent onto a substrate using a high frequency droplet dispenser to form a dosage form, wherein the viscosity of the formulation is less than 3 cP.

2. The method of claim 1, wherein the high frequency droplet dispenser deposits the plurality of droplets using a thermal process.

3. The method of claim 1, wherein the high frequency droplet dispenser deposits the plurality of droplets using a piezoelectric process.

4. The method of claim 1, wherein the high frequency droplet dispenser comprises about 100 to about 1000 individual nozzles.

5. The method of claim 4, wherein each nozzle comprises an aperture with a diameter of about 20 μm to about 120 μm.

6. The method of claim 1, wherein the high frequency droplet dispenser is a high frequency, sub-nanoliter droplet dispenser.

7. The method of claim 6, wherein the high frequency, sub-nanoliter droplet dispenser dispenses droplets at more than 3 kHz as measured per nozzle.

8. The method of claim 1, wherein the high frequency droplet dispenser dispenses droplets with a volume of 1 nanoliter or less.

9. The method of claim 8, wherein the average volume of each dispensed droplet of the plurality of droplets of the formulation is about 1 picoliter to about 50 picoliters.

10. The method of claim 7, wherein the plurality of formulation droplets are deposited onto the substrate according to an average number of droplets per square inch.

11-183. (canceled)
